SEQUENCE LISTING (243 sequences)

SEQ ID NO: 1 - protein, 32 aa, homo sapiens
EDPQGDAAQKTDTSHHDQDHPTFNKITPNLAE

SEQ ID NO: 2 - protein, 23 aa, homo sapiens
FFLPDEGKLQHLENELTHDIITK

SEQ ID NO: 3 - protein, 14 aa, homo sapiens
TIDEKGTEAAGAMF

SEQ ID NO: 4 - protein, 17 aa, homo sapiens
EAIPMSIPPEVKFNKPF

SEQ ID NO: 5 - protein, 18 aa, homo sapiens
EAIPMSIPPEVKFNKPFV

SEQ ID NO: 6 - protein, 21 aa, homo sapiens
MIEQNTKSPLFMGKVVNPTQK

SEQ ID NO: 7 - protein, 18 aa, homo sapiens
DAHKSEVAHRFKDLGEEN

SEQ ID NO: 8 - protein, 8 aa, homo sapiens
YVVHTNYD

SEQ ID NO: 9 - protein, 13 aa, homo sapiens
LGPHAGDVEGHLS

SEQ ID NO: 10 - protein, 17 aa, homo sapiens
TGLSMDGGGSPKGDVDP

SEQ ID NO: 11 - protein, 17 aa, homo sapiens
TGLSMDGGGSPKGDVDP

SEQ ID NO: 12 - protein, 16 aa, homo sapiens
GLSMDGGGSPKGDVDP

SEQ ID NO: 13 - protein, 16 aa, homo sapiens
GLSMDGGGSPKGDVDP

SEQ ID NO: 14 - protein, 12 aa, homo sapiens
DGGGSPKGDVDP

SEQ ID NO: 15 - protein, 12 aa, homo sapiens
YLGGSPKGDVDP

SEQ ID NO: 16 - protein, 23 aa, homo sapiens
LLKNGERIEKVEHSDLSFSKDWS

SEQ ID NO: 17 - protein, 13 aa, homo sapiens
VEHSDLSFSKDWS

SEQ ID NO: 18 - protein, 9 aa, homo sapiens
DFDDFNLED

SEQ ID NO: 19 - protein, 24 aa, homo sapiens
DGVSGGEGKGGSDGGGSHRKEGEE

SEQ ID NO: 20 - protein, 33 aa, homo sapiens
DGVSGGEGKGGSDGGGSHRKEGEEADAPGVIPG

SEQ ID NO: 21 - protein, 19 aa, homo sapiens
SHTSDSDVPSGVTEVVVKL

SEQ ID NO: 22 - protein, 36 aa, homo sapiens
  MISC_FEATURE (3)..(3): Hydroxyproline
GPXGESGREGAXGAEGSXGRDGSXGAKGDRGETGPA

SEQ ID NO: 23 - protein, 34 aa, homo sapiens
  misc_feature (2)..(2): Hydroxyproline
PXGESGREGAPGAEGSXGRDGSXGAKGDRGETGP

SEQ ID NO: 24 - protein, 35 aa, homo sapiens
  misc_feature (1)..(1): Hydroxyproline
XPGESGREGAXGAEGSXGRDGSXGAKGDRGETGPA

SEQ ID NO: 25 - protein, 31 aa, homo sapiens
  misc_feature (14)..(14): Hydroxyproline
ESGREGAPGAEGSXGRDGSXGAKGDRGETGP

SEQ ID NO: 26 - protein, 32 aa, homo sapiens
  misc_feature (8)..(8): Hydroxyproline
ESGREGAXGAEGSXGRDGSXGAKGDRGETGPA

SEQ ID NO: 27 - protein, 23 aa, homo sapiens
  misc_feature (5)..(5): Hydroxyproline
AEGSXGRDGSXGAKGDRGETGPA

SEQ ID NO: 28 - protein, 17 aa, homo sapiens
  misc_feature (4)..(4): Hydroxyproline
EGSXGRDGSXGAKGDRG

SEQ ID NO: 29 - protein, 19 aa, homo sapiens
  misc_feature (4)..(4): Hydroxyproline
EGSXGRDGSXGAKGDRGET

SEQ ID NO: 30 - protein, 22 aa, homo sapiens
  misc_feature (4)..(4): Hydroxyproline
EGSXGRDGSXGAKGDRGETGPA

SEQ ID NO: 31 - protein, 19 aa, homo sapiens
  misc_feature (2)..(2): Hydroxyproline
SXGRDGSXGAKGDRGETGP

SEQ ID NO: 32 - protein, 37 aa, homo sapiens
  misc_feature (11)..(11): Hydroxyproline
DRGETGPAGPXGAXGAPGAPGPVGXAGKSGDRGETGP

SEQ ID NO: 33 - protein, 30 aa, homo sapiens
  misc_feature (4)..(4): Hydroxyproline
AGPXGAXGAXGAXGPVGPAGKSGDRGETGP

SEQ ID NO: 34 - protein, 12 aa, homo sapiens
DKGETGEQGDRG

SEQ ID NO: 35 - protein, 12 aa, homo sapiens
  misc_feature (2)..(2): Hydroxyproline
PXGSAGAPGKDG

SEQ ID NO: 36 - protein, 25 aa, homo sapiens
  misc_feature (15)..(15): Hydroxyproline
GRTGDAGPVGPPGPXGXXGXPGPPS

SEQ ID NO: 37 - protein, 17 aa, homo sapiens
  misc_feature (4)..(4): Hydroxyproline
VGPXGXPGPPGPPGPPS

SEQ ID NO: 38 - protein, 17 aa, homo sapiens
  misc_feature (4)..(4): Hydroxyproline
VGPXGPXGPXGPPGPPS

SEQ ID NO: 39 - protein, 16 aa, homo sapiens
  misc_feature (5)..(6): Hydroxyproline
GPPGXXGPXGPPGPPS

SEQ ID NO: 40 - protein, 13 aa, homo sapiens
  misc_feature (6)..(6): Hydroxyproline
GPPGPXGXXGPPS

SEQ ID NO: 41 - protein, 13 aa, homo sapiens
  misc_feature (6)..(6): Hydroxyproline
GLPGPXGPXGPXG

SEQ ID NO: 42 - protein, 11 aa, homo sapiens
  misc_feature (7)..(7): Hydroxyproline
MGPRGPXGPXG

SEQ ID NO: 43 - protein, 19 aa, homo sapiens
  misc_feature (3)..(3): Hydroxyproline
GPXGPXGKNGDDGEAGKXG

SEQ ID NO: 44 - protein, 22 aa, homo sapiens
  misc_feature (3)..(3): Hydroxyproline
GPXGPXGKNGDDGEAGKXGRPG

SEQ ID NO: 45 - protein, 18 aa, homo sapiens
  misc_feature (2)..(2): Hydroxyproline
PXGPPGKNGDDGEAGKXG

SEQ ID NO: 46 - protein, 16 aa, homo sapiens
  misc_feature (3)..(3): Hydroxyproline
GPXGKNGDDGEAGKXG

SEQ ID NO: 47 - protein, 27 aa, homo sapiens
  misc_feature (3)..(3): Hydroxyproline
GPXGKNGDDGEAGKPGRXGERGPXGXQ

SEQ ID NO: 48 - protein, 15 aa, homo sapiens
  misc_feature (2)..(2): Hydroxyproline
PXGKNGDDGEAGKXG

SEQ ID NO: 49 - protein, 24 aa, homo sapiens
  misc_feature (15)..(15): Hydroxyproline
GKNGDDGEAGKPGRXGERGPXGPQ

SEQ ID NO: 50 - protein, 24 aa, homo sapiens
  misc_feature (12)..(12): Hydroxyproline
GKNGDDGEAGKXGRXGERGPXGPQ

SEQ ID NO: 51 - protein, 23 aa, homo sapiens
  misc_feature (11)..(11): Hydroxyproline
KNGDDGEAGKXGRXGERGPPGXQ

SEQ ID NO: 52 - protein, 22 aa, homo sapiens
  misc_feature (13)..(13): Hydroxyproline
NGDDGEAGKPGRXGERGPXGPQ

SEQ ID NO: 53 - protein, 20 aa, homo sapiens
  misc_feature (12)..(12): Hydroxyproline
GDDGEAGKPGRXGERGPXGP

SEQ ID NO: 54 - protein, 21 aa, homo sapiens
  misc_feature (9)..(9): Hydroxyproline
GDDGEAGKXGRXGERGPXGPQ

SEQ ID NO: 55 - protein, 12 aa, homo sapiens
  misc_feature (8)..(8): Hydroxyproline
DDGEAGKXGRXG

SEQ ID NO: 56 - protein, 19 aa, homo sapiens
  misc_feature (16)..(16): Hydroxyproline
DDGEAGKPGRPGERGXPGX

SEQ ID NO: 57 - protein, 18 aa, homo sapiens
  misc_feature (7)..(7): Hydroxyproline
DGEAGKXGRPGERGPXGP

SEQ ID NO: 58 - protein, 27 aa, homo sapiens
  misc_feature (17)..(17): Hydroxyproline
LDGAKGDAGPAGPKGEXGSXGENGAXG

SEQ ID NO: 59 - protein, 27 aa, homo sapiens
  misc_feature (10)..(10): Hydroxyproline
LDGAKGDAGXAGPKGEXGSXGENGAXG

SEQ ID NO: 60 - protein, 21 aa, homo sapiens
  misc_feature (11)..(11): Hydroxyproline
DAGPAGPKGEXGSXGENGAXG

SEQ ID NO: 61 - protein, 15 aa, homo sapiens
  misc_feature (2)..(2): Hydroxyproline
SXGENGAXGQMGPRG

SEQ ID NO: 62 - protein, 15 aa, homo sapiens
  misc_feature (2)..(2): Hydroxyproline
SXGENGAXGQMGPRG

SEQ ID NO: 63 - protein, 25 aa, homo sapiens
  misc_feature (6)..(6): Hydroxyproline
GNSGEXGAXGSKGDTGAKGEXGPVG

SEQ ID NO: 64 - protein, 20 aa, homo sapiens
  misc_feature (5)..(5): Hydroxyproline
NSGEXGAXGSKGDTGAKGEX

SEQ ID NO: 65 - protein, 22 aa, homo sapiens
  misc_feature (5)..(5): Hydroxyproline
NSGEXGAXGSKGDTGAKGEXGP

SEQ ID NO: 66 - protein, 23 aa, homo sapiens
  misc_feature (4)..(4): Hydroxyproline
SGEXGAXGSKGDTGAKGEXGPVG

SEQ ID NO: 67 - protein, 22 aa, homo sapiens
  misc_feature (3)..(3): Hydroxyproline
GEXGAXGSKGDTGAKGEXGPVG

SEQ ID NO: 68 - protein, 18 aa, homo sapiens
  misc_feature (2)..(2): Hydroxyproline
AXGSKGDTGAKGEXGPVG

SEQ ID NO: 69 - protein, 9 aa, homo sapiens
  misc_feature (2)..(2): Hydroxyproline
SXGEAGRXG

SEQ ID NO: 70 - protein, 37 aa, homo sapiens
  misc_feature (14)..(14): Hydroxyproline
SPGEAGRPGEAGLXGAKGLTGSPGSPGXDGKTGPPGP

SEQ ID NO: 71 - protein, 33 aa, homo sapiens
  misc_feature (5)..(5): Hydroxyproline
LTGSXGSXGXDGKTGPPGPAGQDGRPGPXGXXG

SEQ ID NO: 72 - protein, 18 aa, homo sapiens
  misc_feature (4)..(4): Hydroxyproline
TGSXGSXGPDGKTGPXGP

SEQ ID NO: 73 - protein, 20 aa, homo sapiens
  misc_feature (4)..(4): Hydroxyproline
TGSXGSXGPDGKTGPPGXAG

SEQ ID NO: 74 - protein, 17 aa, homo sapiens
  misc_feature (3)..(3): Hydroxyproline
GSXGSXGPDGKTGPPGX

SEQ ID NO: 75 - protein, 14 aa, homo sapiens
  misc_feature (2)..(2): Hydroxyproline
SXGSXGPDGKTGPX

SEQ ID NO: 76 - protein, 11 aa, homo sapiens
  misc_feature (2)..(2): Hydroxyproline
SXGPDGKTGPX

SEQ ID NO: 77 - protein, 14 aa, homo sapiens
  misc_feature (2)..(2): Hydroxyproline
SXGPDGKTGPXGPA

SEQ ID NO: 78 - protein, 11 aa, homo sapiens
  misc_feature (9)..(9): Hydroxyproline
GPDGKTGPXGP

SEQ ID NO: 79 - protein, 12 aa, homo sapiens
  misc_feature (9)..(9): Hydroxyproline
GPDGKTGPXGPA

SEQ ID NO: 80 - protein, 10 aa, homo sapiens
  misc_feature (7)..(7): Hydroxyproline
DGKTGPXGPA

SEQ ID NO: 81 - protein, 23 aa, homo sapiens
  misc_feature (6)..(6): Hydroxyproline
DGKTGXPGPAGQDGRPGPXGXXG

SEQ ID NO: 82 - protein, 13 aa, homo sapiens
  misc_feature (6)..(6): Hydroxyproline
GQDGRXGPXGPXG

SEQ ID NO: 83 - protein, 12 aa, homo sapiens
  misc_feature (4)..(4): Hydroxyproline
DGRXGPXGPXGA

SEQ ID NO: 84 - protein, 10 aa, homo sapiens
  misc_feature (3)..(3): Hydroxyproline
GRXGPXGPXG

SEQ ID NO: 85 - protein, 37 aa, homo sapiens
  misc_feature (14)..(14): Hydroxyproline
AAGEPGKAGERGVXGXXGAVGPAGKDGEAGAQGPXGP

SEQ ID NO: 86 - protein, 30 aa, homo sapiens
  misc_feature (11)..(11): Hydroxyproline
KDGEAGAQGPXGPAGPAGERGEQGPAGSXG

SEQ ID NO: 87 - protein, 45 aa, homo sapiens
  misc_feature (11)..(11): Hydroxyproline
ERGEQGPAGSXGFQGLXGXAGXXGEAGKXGEQGVPGDLGAPGPSG

SEQ ID NO: 88 - protein, 13 aa, homo sapiens
  misc_feature (3)..(3): Hydroxyproline
GPXGEAGKXGEQG

SEQ ID NO: 89 - protein, 19 aa, homo sapiens
  misc_feature (3)..(3): Hydroxyproline
GPXGEAGKXGEQGVPGDLG

SEQ ID NO: 90 - protein, 19 aa, homo sapiens
  misc_feature (3)..(3): Hydroxyproline
GPXGEAGKXGEQGVXGDLG

SEQ ID NO: 91 - protein, 23 aa, homo sapiens
  misc_feature (3)..(3): Hydroxyproline
GPXGEAGKXGEQGVXGDLGAXGP

SEQ ID NO: 92 - protein, 18 aa, homo sapiens
  misc_feature (2)..(2): Hydroxyproline
PXGEAGKXGEQGVPGDLG

SEQ ID NO: 93 - protein, 18 aa, homo sapiens
  misc_feature (1)..(1): Hydroxyproline
XPGEAGKXGEQGVXGDLG

SEQ ID NO: 94 - protein, 22 aa, homo sapiens
  misc_feature (2)..(2): Hydroxyproline
PXGEAGKXGEQGVXGDLGAPGP

SEQ ID NO: 95 - protein, 22 aa, homo sapiens
  misc_feature (2)..(2): Hydroxyproline
PXGEAGKXGEQGVXGDLGAXGP

SEQ ID NO: 96 - protein, 12 aa, homo sapiens
  misc_feature (2)..(2): Hydroxyproline
KXGEQGVXGDLG

SEQ ID NO: 97 - protein, 18 aa, homo sapiens
  misc_feature (2)..(2): Hydroxyproline
KXGEQGVXGDLGAXGPSG

SEQ ID NO: 98 - protein, 28 aa, homo sapiens
  misc_feature (6)..(6): Hydroxyproline
GANGAXGNDGAKGDAGAXGAXGSQGAXG

SEQ ID NO: 99 - protein, 20 aa, homo sapiens
  misc_feature (4)..(4): Hydroxyproline
NGAXGNDGAKGDAGAXGAXG

SEQ ID NO: 100 - protein, 26 aa, homo sapiens
  misc_feature (4)..(4): Hydroxyproline
NGAXGNDGAKGDAGAXGAXGSQGAXG

SEQ ID NO: 101 - protein, 26 aa, homo sapiens
  misc_feature (4)..(4): Hydroxyproline
NGAXGNDGAKGDAGAXGAXGSQGAXG

SEQ ID NO: 102 - protein, 25 aa, homo sapiens
  misc_feature (3)..(3): Hydroxyproline
GAXGNDGAKGDAGAXGAXGSQGAXG

SEQ ID NO: 103 - protein, 30 aa, homo sapiens
  misc_feature (10)..(10): Hydroxyproline
LTGPIGPPGXAGAXGDKGESGPSGPAGPTG

SEQ ID NO: 104 - protein, 26 aa, homo sapiens
  misc_feature (4)..(4): Hydroxyproline
IGPXGPAGAXGDKGESGPSGPAGPTG

SEQ ID NO: 105 - protein, 11 aa, homo sapiens
  misc_feature (2)..(2): Hydroxyproline
AXGDKGESGPS

SEQ ID NO: 106 - protein, 11 aa, homo sapiens
  misc_feature (2)..(2): Hydroxyproline
AXGDRGEXGPP

SEQ ID NO: 107 - protein, 11 aa, homo sapiens
  misc_feature (2)..(2): Hydroxyproline
AXGDRGEXGXP

SEQ ID NO: 108 - protein, 14 aa, homo sapiens
  misc_feature (2)..(2): Hydroxyproline
AXGDRGEXGPXGPA

SEQ ID NO: 109 - protein, 11 aa, homo sapiens
  misc_feature (5)..(5): Hydroxyproline
DRGEXGPXGPA

SEQ ID NO: 110 - protein, 29 aa, homo sapiens
  misc_feature (3)..(3): Hydroxyproline
GPXGADGQXGAKGEXGDAGAKGDAGXPGP

SEQ ID NO: 111 - protein, 30 aa, homo sapiens
  misc_feature (3)..(3): Hydroxyproline
GPXGADGQXGAKGEXGDAGAKGDAGXPGPA

SEQ ID NO: 112 - protein, 32 aa, homo sapiens
  misc_feature (3)..(3): Hydroxyproline
GPXGADGQPGAKGEXGDAGAKGDAGXPGPAGP

SEQ ID NO: 113 - protein, 40 aa, homo sapiens
  misc_feature (3)..(3): Hydroxyproline
GPXGADGQPGAKGEXGDAGAKGDAGXPGPAGPAGPPGPIG

SEQ ID NO: 114 - protein, 31 aa, homo sapiens
  misc_feature (2)..(2): Hydroxyproline
PXGADGQXGAKGEXGDAGAKGDAGPXGPAGP

SEQ ID NO: 115 - protein, 39 aa, homo sapiens
  misc_feature (2)..(2): Hydroxyproline
PXGADGQPGAKGEXGDAGAKGDAGPXGPAGPAGPXGPIG

SEQ ID NO: 116 - protein, 27 aa, homo sapiens
  misc_feature (12)..(12): Hydroxyproline
GADGQPGAKGEXGDAGAKGDAGPXGPA

SEQ ID NO: 117 - protein, 25 aa, homo sapiens
  misc_feature (23)..(23): Hydroxyproline
ADGQPGAKGEPGDAGAKGDAGPXGP

SEQ ID NO: 118 - protein, 26 aa, homo sapiens
  misc_feature (11)..(11): Hydroxyproline
ADGQPGAKGEXGDAGAKGDAGPXGPA

SEQ ID NO: 119 - protein, 26 aa, homo sapiens
  misc_feature (5)..(5): Hydroxyproline
ADGQXGAKGEXGDAGAKGDAGPXGPA

SEQ ID NO: 120 - protein, 26 aa, homo sapiens
  misc_feature (5)..(5): Hydroxyproline
ADGQXGAKGEXGDAGAKGDAGXXGPA

SEQ ID NO: 121 - protein, 28 aa, homo sapiens
  misc_feature (11)..(11): Hydroxyproline
ADGQPGAKGEXGDAGAKGDAGPXGPAGP

SEQ ID NO: 122 - protein, 28 aa, homo sapiens
  misc_feature (5)..(5): Hydroxyproline
ADGQXGAKGEXGDAGAKGDAGXPGPAGP

SEQ ID NO: 123 - protein, 36 aa, homo sapiens
  misc_feature (28)..(28): Hydroxyproline
ADGQPGAKGEPGDAGAKGDAGPPGPAGXAGXPGPIG

SEQ ID NO: 124 - protein, 36 aa, homo sapiens
  misc_feature (5)..(5): Hydroxyproline
ADGQXGAKGEXGDAGAKGDAGXPGPAGPAGPPGXIG

SEQ ID NO: 125 - protein, 24 aa, homo sapiens
  misc_feature (10)..(10): Hydroxyproline
DGQPGAKGEXGDAGAKGDAGPPGX

SEQ ID NO: 126 - protein, 24 aa, homo sapiens
  misc_feature (4)..(4): Hydroxyproline
DGQXGAKGEXGDAGAKGDAGPPGX

SEQ ID NO: 127 - protein, 17 aa, homo sapiens
  misc_feature (2)..(2): Hydroxyproline
EXGDAGAKGDAGPXGPA

SEQ ID NO: 128 - protein, 28 aa, homo sapiens
  misc_feature (17)..(17): Hydroxyproline
KEGGKGPRGETGPAGRXGEVGXPGPXGP

SEQ ID NO: 129 - protein, 30 aa, homo sapiens
  misc_feature (17)..(17): Hydroxyproline
KEGGKGPRGETGPAGRXGEVGXPGPXGPAG

SEQ ID NO: 130 - protein, 21 aa, homo sapiens
  misc_feature (8)..(8): Hydroxyproline
ETGPAGRXGEVGPXGPXGPAG

SEQ ID NO: 131 - protein, 29 aa, homo sapiens
  misc_feature (4)..(4): Hydroxyproline
QGPXGPSGEEGKRGPNGEAGSAGPXGPXG

SEQ ID NO: 132 - protein, 13 aa, homo sapiens
  misc_feature (3)..(3): Hydroxyproline
GPXGPDGNKGEXG

SEQ ID NO: 133 - protein, 18 aa, homo sapiens
  misc_feature (2)..(2): Hydroxyproline
GXAGXAGPRGSXGERGEV

SEQ ID NO: 134 - protein, 40 aa, homo sapiens
  misc_feature (17)..(17): Hydroxyproline
DQGPVGRTGEVGAVGPXGFAGEKGPSGEAGTAGXPGTXGP

SEQ ID NO: 135 - protein, 42 aa, homo sapiens
  misc_feature (17)..(17): Hydroxyproline
DQGPVGRTGEVGAVGPXGFAGEKGPSGEAGTAGPXGTXGPQG

SEQ ID NO: 136 - protein, 36 aa, homo sapiens
  misc_feature (10)..(10): Hydroxyproline
RTGEVGAVGXPGFAGEKGPSGEAGTAGPPGTXGXQG

SEQ ID NO: 137 - protein, 36 aa, homo sapiens; identical to SEQ ID NO: 136
  misc_feature (10)..(10): Hydroxyproline

SEQ ID NO: 138 - protein, 33 aa, homo sapiens
  misc_feature (10)..(10): Hydroxyproline
TGEVGAVGPXGFAGEKGPSGEAGTAGPXGTXGP

SEQ ID NO: 139 - protein, 20 aa, homo sapiens
  misc_feature (15)..(15): Hydroxyproline
QGEAGQKGDAGAPGXQGXSG

SEQ ID NO: 140 - protein, 30 aa, homo sapiens
  misc_feature (8)..(8): Hydroxyproline
EAGRDGNXGNDGPXGRDGQXGHKGERGYPG

SEQ ID NO: 141 - protein, 28 aa, homo sapiens
  misc_feature (6)..(6): Hydroxyproline
GRDGNXGNDGPXGRDGQXGHKGERGYXG

SEQ ID NO: 142 - protein, 21 aa, homo sapiens
  misc_feature (5)..(5): Hydroxyproline
NDGPXGRDGQXGHKGERGYXG

SEQ ID NO: 143 - protein, 14 aa, homo sapiens
  misc_feature (4)..(4): Hydroxyproline
DGPXGRDGQXGHKG

SEQ ID NO: 144 - protein, 14 aa, homo sapiens
  misc_feature (2)..(2): Hydroxyproline
AXGEDGRXGPXGPQ

SEQ ID NO: 145 - protein, 13 aa, homo sapiens
  misc_feature (1)..(1): Hydroxyproline
XPGSNGNXGPXGP

SEQ ID NO: 146 - protein, 25 aa, homo sapiens
  misc_feature (2)..(2): Hydroxyproline
AXGHXGPXGPVGPAGKSGDRGESGP

SEQ ID NO: 147 - protein, 28 aa, homo sapiens
  misc_feature (14)..(14): Hydroxyproline
NRGERGSEGSPGHXGQXGXXGXPGAPGP

SEQ ID NO: 148 - protein, 28 aa, homo sapiens
  misc_feature (11)..(11): Hydroxyproline
NRGERGSEGSXGHXGQXGXXGPPGAPGX

SEQ ID NO: 149 - protein, 22 aa, homo sapiens
  misc_feature (5)..(5): Hydroxyproline
SEGSXGHXGQXGXPGPPGAXGX

SEQ ID NO: 150 - protein, 23 aa, homo sapiens
  misc_feature (3)..(3): Hydroxyproline
AGXPGPPGXXGTSGHXGSXGSXG

SEQ ID NO: 151 - protein, 19 aa, homo sapiens
  misc_feature (2)..(2): Hydroxyproline
GXPGPXGTSGHXGSXGSXG

SEQ ID NO: 152 - protein, 48 aa, homo sapiens
  misc_feature (14)..(14): Hydroxyproline
ARGNDGARGSDGQXGXXGPPGTAGFPGSXGAKGEVGXAGSXGSNGAXG

SEQ ID NO: 153 - protein, 48 aa, homo sapiens
  misc_feature (14)..(14): Hydroxyproline
ARGNDGARGSDGQXGXPGPXGTAGFXGSXGAKGEVGXAGSXGSNGAXG

SEQ ID NO: 154 - protein, 24 aa, homo sapiens
  misc_feature (5)..(5): Hydroxyproline
SDGQXGPXGPXGTAGFXGSXGAKG

SEQ ID NO: 155 - protein, 30 aa, homo sapiens
  misc_feature (8)..(8): Hydroxyproline
ERGEAGIXGVXGAKGEDGKDGSXGEXGANG

SEQ ID NO: 156 - protein, 15 aa, homo sapiens
  misc_feature (2)..(2): Hydroxyproline
SXGGXGSDGKXGPXG

SEQ ID NO: 157 - protein, 13 aa, homo sapiens
  misc_feature (3)..(3): Hydroxyproline
GGXGSDGKXGPXG

SEQ ID NO: 158 - protein, 12 aa, homo sapiens
  misc_feature (2)..(2): Hydroxyproline
GXGSDGKXGPXG

SEQ ID NO: 159 - protein, 21 aa, homo sapiens
  misc_feature (7)..(7): Hydroxyproline
KGNDGAXGKNGERGGXGGXGP

SEQ ID NO: 160 - protein, 20 aa, homo sapiens
  misc_feature (6)..(6): Hydroxyproline
GNDGAXGKNGERGGXGGXGP

SEQ ID NO: 161 - protein, 17 aa, homo sapiens
  misc_feature (5)..(5): Hydroxyproline
NDGAXGKNGERGGXGGX

SEQ ID NO: 162 - protein, 19 aa, homo sapiens
  misc_feature (5)..(5): Hydroxyproline
NDGAXGKNGERGGXGGXGP

SEQ ID NO: 163 - protein, 18 aa, homo sapiens
  misc_feature (4)..(4): Hydroxyproline
DGAXGKNGERGGXGGXGP

SEQ ID NO: 164 - protein, 28 aa, homo sapiens
  misc_feature (26)..(26): Hydroxyproline
KNGETGPQGPPGPTGPGGDKGDTGPXGP

SEQ ID NO: 165 - protein, 28 aa, homo sapiens
  misc_feature (11)..(11): Hydroxyproline
KNGETGPQGPXGPTGPGGDKGDTGPPGX

SEQ ID NO: 166 - protein, 30 aa, homo sapiens
  misc_feature (26)..(26): Hydroxyproline
KNGETGPQGPPGPTGPGGDKGDTGPXGPQG

SEQ ID NO: 167 - protein, 30 aa, homo sapiens
  misc_feature (26)..(26): Hydroxyproline
KNGETGPQGPPGPTGPGGDKGDTGPXGXQG

SEQ ID NO: 168 - protein, 15 aa, homo sapiens
  misc_feature (13)..(13): Hydroxyproline
TGPGGDKGDTGPXGP

SEQ ID NO: 169 - protein, 14 aa, homo sapiens
  misc_feature (12)..(12): Hydroxyproline
GPGGDKGDTGPXGP

SEQ ID NO: 170 - protein, 9 aa, homo sapiens
  misc_feature (7)..(7): Hydroxyproline
KGDTGPXGP

SEQ ID NO: 171 - protein, 24 aa, homo sapiens
  misc_feature (5)..(5): Hydroxyproline
LQGLXGTGGPXGENGKXGEXGPKG

SEQ ID NO: 172 - protein, 48 aa, homo sapiens
  misc_feature (5)..(5): Hydroxyproline
LQGLXGTGGXXGENGKXGEXGXKGDAGAPGAPGGKGDAGAPGERGXXG

SEQ ID NO: 173 - protein, 16 aa, homo sapiens
  misc_feature (3)..(3): Hydroxyproline
GLXGTGGPXGENGKXG

SEQ ID NO: 174 - protein, 20 aa, homo sapiens
  misc_feature (3)..(3): Hydroxyproline
GLXGTGGPXGENGKPGEPGX

SEQ ID NO: 175 - protein, 20 aa, homo sapiens
  misc_feature (3)..(3): Hydroxyproline
GLXGTGGPXGENGKXGEPGX

SEQ ID NO: 176 - protein, 25 aa, homo sapiens
  misc_feature (6)..(6): Hydroxyproline
GDAGAXGAXGGKGDAGAXGERGPXG

SEQ ID NO: 177 - protein, 24 aa, homo sapiens
  misc_feature (5)..(5): Hydroxyproline
DAGAXGAPGGKGDAGAXGERGPXG

SEQ ID NO: 178 - protein, 24 aa, homo sapiens
  misc_feature (5)..(5): Hydroxyproline
DAGAXGAXGGKGDAGAXGERGPXG

SEQ ID NO: 179 - protein, 21 aa, homo sapiens
  misc_feature (2)..(2): Hydroxyproline
AXGAPGGKGDAGAXGERGPXG

SEQ ID NO: 180 - protein, 21 aa, homo sapiens
  misc_feature (2)..(2): Hydroxyproline
AXGAXGGKGDAGAXGERGPXG

SEQ ID NO: 181 - protein, 18 aa, homo sapiens
  misc_feature (2)..(2): Hydroxyproline
AXGGKGDAGAXGERGPXG

SEQ ID NO: 182 - protein, 30 aa, homo sapiens
  misc_feature (8)..(8): Hydroxyproline
LRGGAGPXGPEGGKGAAGPXGPXGAAGTXG

SEQ ID NO: 183 - protein, 33 aa, homo sapiens
  misc_feature (7)..(7): Hydroxyproline
LRGGAGXPGPEGGKGAAGXPGXXGAAGTPGLQG

SEQ ID NO: 184 - protein, 13 aa, homo sapiens
DGVPGKDGPRGPT

SEQ ID NO: 185 - protein, 11 aa, homo sapiens
  misc_feature (2)..(2): Hydroxyproline
SXGERGETGPX

SEQ ID NO: 186 - protein, 13 aa, homo sapiens
  misc_feature (2)..(2): Hydroxyproline
SXGERGETGPXGP

SEQ ID NO: 187 - protein, 14 aa, homo sapiens
  misc_feature (2)..(2): Hydroxyproline
SXGERGETGPXGPA

SEQ ID NO: 188 - protein, 34 aa, homo sapiens
  misc_feature (14)..(14): Hydroxyproline
QNGEPGGKGERGAXGEKGEGGXXGVAGPXGGSGP

SEQ ID NO: 189 - protein, 22 aa, homo sapiens
  misc_feature (12)..(12): Hydroxyproline
GEPGGKGERGAXGEKGEGGXPG

SEQ ID NO: 190 - protein, 35 aa, homo sapiens
  misc_feature (6)..(6): Hydroxyproline
GERGSXGGXGAAGFXGARGLXGXPGSNGNPGPXGX

SEQ ID NO: 191 - protein, 22 aa, homo sapiens
  misc_feature (5)..(5): Hydroxyproline
SNGNXGPXGPSGSXGKDGPXGP

SEQ ID NO: 192 - protein, 24 aa, homo sapiens
  misc_feature (5)..(5): Hydroxyproline
SNGNXGXPGPSGSPGKDGPXGXAG

SEQ ID NO: 193 - protein, 37 aa, homo sapiens
  misc_feature (5)..(5): Hydroxyproline
NTGAXGSXGVSGXKGDAGQXGEKGSPGAQGPPGAPGX

SEQ ID NO: 194 - protein, 19 aa, homo sapiens
  misc_feature (9)..(9): Hydroxyproline
GPPGFTGPXGPXGPPGPPG

SEQ ID NO: 195 - protein, 12 aa, homo sapiens
PIGQEGAPGRPG

SEQ ID NO: 196 - protein, 12 aa, homo sapiens
  misc_feature (1)..(1): Hydroxyproline
XGPPGXPGXPSP

SEQ ID NO: 197 - protein, 33 aa, homo sapiens
  misc_feature (5)..(5): Hydroxyproline
LTGNXGVQGPEGKLGPLGAXGEDGRXGXPGSIG

SEQ ID NO: 198 - protein, 17 aa, homo sapiens
  misc_feature (1)..(2): Hydroxyproline
XXGDSGXXGEKGDPGRP

SEQ ID NO: 199 - protein, 13 aa, homo sapiens
  misc_feature (3)..(3): Hydroxyproline
GPXGPXGPPGPXA

SEQ ID NO: 200 - protein, 20 aa, homo sapiens
  misc_feature (2)..(2): Hydroxyproline
GXPGEGRAGEXGTAGXTGXP

SEQ ID NO: 201 - protein, 13 aa, homo sapiens
  misc_feature (3)..(3): Hydroxyproline
GPXGPXGPXGPVT

SEQ ID NO: 202 - protein, 15 aa, homo sapiens
  misc_feature (6)..(6): Hydroxyproline
GLPGPXGPXGSFLSN

SEQ ID NO: 203 - protein, 15 aa, homo sapiens
  misc_feature (2)..(2): Hydroxyproline
PXGPXGPXGPPGTPV

SEQ ID NO: 204 - protein, 22 aa, homo sapiens
DDILASPPRLPEPQPYPGAPHH

SEQ ID NO: 205 - protein, 24 aa, homo sapiens
DDILASPPRLPEPQPYPGAPHHSS

SEQ ID NO: 206 - protein, 15 aa, homo sapiens
  misc_feature (7)..(7): Hydroxyproline
KGEAGLXGAXGSPGQ

SEQ ID NO: 207 - protein, 14 aa, homo sapiens
DEAGSEADHEGTHS

SEQ ID NO: 208 - protein, 16 aa, homo sapiens
DEAGSEADHEGTHSTK

SEQ ID NO: 209 - protein, 17 aa, homo sapiens
DEAGSEADHEGTHSTKR

SEQ ID NO: 210 - protein, 18 aa, homo sapiens
EEAPSLRPAPPPISGGGY

SEQ ID NO: 211 - protein, 21 aa, homo sapiens
ELTETGVEAAAASAISVARTL

SEQ ID NO: 212 - protein, 15 aa, homo sapiens
FIFPPSDEQLKSGTA

SEQ ID NO: 213 - protein, 15 aa, homo sapiens
IFPPSDEQLKSGTAS

SEQ ID NO: 214 - protein, 10 aa, homo sapiens
ATKTVGSDTF

SEQ ID NO: 215 - protein, 9 aa, homo sapiens
TISRLEPED

SEQ ID NO: 216 - protein, 24 aa, homo sapiens
MASDASHALEAALEQMDGIIAGTK

SEQ ID NO: 217 - protein, 18 aa, homo sapiens
FGASAGTGDLSDNHDIIS

SEQ ID NO: 218 - protein, 26 aa, homo sapiens
IPVKQADSGSSEEKQLYNKYPDAVAT

SEQ ID NO: 219 - protein, 13 aa, homo sapiens
YKRKANDESNEHS

SEQ ID NO: 220 - protein, 10 aa, homo sapiens
YNKYPDAVAT

SEQ ID NO: 221 - protein, 15 aa, homo sapiens
DAPGQYGAYFHDDGF

SEQ ID NO: 222 - protein, 13 aa, homo sapiens
FHDDGFLAFPGHV

SEQ ID NO: 223 - protein, 15 aa, homo sapiens
SGDSDDDEPPPLPRL

SEQ ID NO: 224 - protein, 14 aa, homo sapiens
GDSDDDEPPPLPRL

SEQ ID NO: 225 - protein, 35 aa, homo sapiens
FAEEKAVADTRDQADGSRASVDSGSSEEQGGSSRA

SEQ ID NO: 226 - protein, 14 aa, homo sapiens
DSGSSEEQGGSSRA

SEQ ID NO: 227 - protein, 17 aa, homo sapiens
YSQGSKGPGEDFRMATL

SEQ ID NO: 228 - protein, 17 aa, homo sapiens
DHDVGSELPPEGVLGAL

SEQ ID NO: 229 - protein, 17 aa, homo sapiens
SVDETGQMSATAKGRVR

SEQ ID NO: 230 - protein, 9 aa, homo sapiens
VIEHIMEDL

SEQ ID NO: 231 - protein, 10 aa, homo sapiens
DTNADKQLSF

SEQ ID NO: 232 - protein, 31 aa, homo sapiens
SSQGGSLPSEEKGHPQEESEESNVSMASLGE

SEQ ID NO: 233 - protein, 9 aa, homo sapiens
SGSVIDQSR

SEQ ID NO: 234 - protein, 10 aa, homo sapiens
SGSVIDQSRV

SEQ ID NO: 235 - protein, 14 aa, homo sapiens
SVIDQSRVLNLGPI

SEQ ID NO: 236 - protein, 15 aa, homo sapiens
SVIDQSRVLNLGPIT

SEQ ID NO: 237 - protein, 17 aa, homo sapiens
SVIDQSRVLNLGPITRK

SEQ ID NO: 238 - protein, 14 aa, homo sapiens
VIDQSRVLNLGPIT

SEQ ID NO: 239 - protein, 15 aa, homo sapiens
VIDQSRVLNLGPITR

SEQ ID NO: 240 - protein, 9 aa, homo sapiens
VLNLGPITR

SEQ ID NO: 241 - protein, 37 aa, homo sapiens
GRPEAQPPPLSSEHKEPVAGDAVPGPKDGSAPEVRGA

SEQ ID NO: 242 - protein, 16 aa, homo sapiens
VGGGEQPPPAPAPRRE

SEQ ID NO: 243 - protein, 28 aa, homo sapiens
PEAEAEAEAGAGGEAAAEEGAAGRKARG

(19) United States
(12) Patent Application Publication
(10) Pub. No.: US 2015/0065391 A1
(43) Pub. Date: Mar. 5, 2015

(54) POLYPEPTIDE MARKERS FOR DIAGNOSIS AND ASSESSMENT OF HEART FAILURE

(71) Applicant: Mosaiques Diagnostics and Therapeutics AG, Hannover (DE)

(72) Inventor: Harald Mischak, Sehnde (DE)

(21) Appl. No.: 14/445,856

(22) PCT Filed: Jan. 30, 2013
(86) PCT No.: PCT/EP2013/051775

§ 371 Date: Jul. 29, 2014

(30) Foreign Application Priority Data
Jan. 30, 2012 (EP) 12153123.0

(51) Int. Cl. G01N 33/68

(52) U.S. Cl. CPC G01N 33/6893; G01N 33/6848; G01N 2800/325
USPC 506/12

(57) ABSTRACT

Method for diagnosis of heart failure, comprising the step of determining the presence or absence or amplitude of at least three polypeptide markers in a urine sample, the polypeptide markers being selected from the markers characterized in Table 1 by values for the molecular masses and the migration time.

### DESCRIPTION OF THE INVENTION

The present invention relates to the use of the presence or absence of one or more peptide markers in a sample from a subject for the diagnosis and assessment of heart failure and to a method for the diagnosis and assessment of heart failure, wherein the presence or absence of the peptide marker or markers is indicative of said heart failure.

Heart failure is a progressive disease starting with risk factors for left-ventricular dysfunction (e.g., hypertension), progressing with asymptomatic change in the heart muscle structure (left-ventricular hypertrophy) and in cardiac function (e.g., reduced relaxation), then becoming a clinically visible heart failure, which may lead to disability and to death.

The five-year mortality rate of symptomatic heart failure is about 60%.

Clinically, heart failure can occur with predominantly systolic (relating to contraction) or diastolic (relating to relaxation) dysfunction, but both may also occur together.

In a group of randomly selected subjects, the frequency of a diastolic left-ventricular dysfunction (early phase) that is asymptomatic, but can be diagnosed by an echocardiogram, is is up to 27%. This is the fraction who are at risk of developing diastolic heart failure. Despite the significant loads on society associated therewith, the diagnosis of heart failure and, in particular, of diastolic left-ventricular heart failure is still difficult. Simply applicable screening techniques are lacking.

When diagnosed already in the asymptomatic stage, treatment with, for example, ACE inhibitors, angiotensin receptor blockers or aldosterone antagonists could be started already at this time, in order to delay or prevent progression to clinical symptoms.

Thus, it is clear that there is a need for a non-invasive possibility of an early and reliable diagnosis of heart failure.

Therefore, the object of the present invention is to provide processes and means for the diagnosis of left-ventricular disorders and diastolic heart failure.

This object is achieved by a process for the diagnosis of heart failure, comprising the step of determining the presence or absence or amplitude of at least three polypeptide markers in a urine sample, wherein said polypeptide markers are selected from the markers characterized in Table 1 by values for the molecular masses and migration times and in some cases their peptide sequence.

TABLE 1
Polypeptide markers

| | Mass | CE Time | | | |
|---|---|---|---|---|---|
| ID | [Da] | [min] | Sequence | Protein name | Entry in data base |
| 912 | 826.201 | 33.4119 | | | |
| 1495 | 838.401 | 35.0611 | | | |
| 1577 | 840.407 | 23.1656 | KGDTGPpGP | Collagen alpha-1(III) | CO3A1_HUMAN (629-637) |
| | | | | chain | |
| 2505 | 858.393 | 23.2367 | SpGEAGRpG | Collagen alpha-1(I) | CO1A1_HUMAN (522-530) |
| | | | | chain | |
| 2659 | 860.362 | 26.1404 | | | |
| 3052 | 868.409 | 23.312 | | | |
| 3472 | 875.477 | 21.9392 | | | |
| 3608 | 879.499 | 19.881 | | | |
| 3743 | 883.41 | 23.2563 | | | |
| 3796 | 884.292 | 43.8066 | | | |
| 4730 | 898.315 | 26.0488 | | | |
| 4845 | 900.269 | 43.6572 | | | |
| 4976 | 902.413 | 20.8458 | | | |
| 5184 | 906.179 | 34.2619 | | | |
| 5661 | 911.265 | 34.3452 | | | |
| 5675 | 911.435 | 25.8763 | DGKTGPpGPA | Collagen alpha-1(I) | CO1A1_HUMAN (550-559) |
| | | | | chain | |
| 6803 | 928.372 | 35.4637 | | | |
| 7408 | 935.447 | 23.6811 | GRpGPpGPpG | Collagen alpha-1(I) | CO1A1_HUMAN (563-572) |
| | | | | chain | |
| 7953 | 940.438 | 25.1411 | | | |
| 8390 | 945.416 | 25.7221 | | | |
| 8503 | 947.473 | 24.741 | SGSVIDQSR | Uromodulin | UROM_HUMAN (589-597) |
| 8695 | 949.219 | 34.3333 | | | |
| 8725 | 949.404 | 25.7932 | | | |
| 9930 | 963.491 | 21.8073 | | | |
| 11282 | 980.5 | 22.4055 | | | |
| 11413 | 981.585 | 24.7955 | VLNLGPITR | Uromodulin | UROM_HUMAN (598-606) |
| 12264 | 992.416 | 20.3967 | | | |
| 12380 | 994.434 | 25.0743 | GPDGKTGPpGP | Collagen alpha-1(I) | CO1A1_HUMAN (548-558) |
| | | | | chain | |
| 12434 | 995.386 | 25.1088 | | | |
| 12993 | 1009.41 | 20.9807 | | | |
| 12998 | 1009.45 | 27.2678 | YVVHTNYD | Alpha-1-microglobulin | AMBP_HUMAN (121-128) |
| 13145 | 1012.5 | 35.0293 | | | |
| 13342 | 1016.45 | 25.7851 | ApGDKGESGPS | Collagen alpha-1(I) | CO1A1_HUMAN (777-787) |
| | | | | chain | |
| 13429 | 1018.46 | 24.5356 | | | |
| 13746 | 1025.47 | 24.9959 | ATKTVGSDTF | Kininogen-1 | KNG1_HUMAN (62-71) |
| 13747 | 1025.46 | 25.5952 | PpGSAGAPGKDG | Collagen alpha-1(I) | CO1A1_HUMAN |
| | | | | chain | (1143-1154) |
| 14047 | 1032.45 | 25.8974 | | | |
| 14071 | 1032.5 | 21.2104 | | | |
| 14478 | 1040.48 | 25.0502 | SpGPDGKTGPp | Collagen alpha-1(I) | CO1A1_HUMAN (546-556) |
| | | | | chain | |
| 14751 | 1046.43 | 27.6437 | | | |
| 14763 | 1046.51 | 25.3545 | SGSVIDQSRV | Uromodulin | UROM_HUMAN (589-598) |
| 14906 | 1050.48 | 26.9248 | MGPRGPpGPpG | Collagen alpha-1(I) | CO1A1_HUMAN (217-227) |
| | | | | chain | |
| 15216 | 1058.48 | 24.893 | TISRLEPED | Ig kappa chain V-III | KV303_HUMAN (79-87) |
| | | | | region NG9 | |
| 15296 | 1060.38 | 38.31 | | | |
| 15561 | 1065.5 | 25.4545 | GPDGKTGPpGPA | Collagen alpha-1(I) | CO1A1_HUMAN (548-559) |
| | | | | chain | |
| 15776 | 1068.45 | 24.7645 | | | |
| 15887 | 1069.47 | 26.332 | GpGSDGKpGPpG | Collagen alpha-1(III) | CO3A1_HUMAN (544-555) |
| | | | | chain | |
| 16059 | 1071.49 | 21.4307 | | | |
| 16168 | 1073.32 | 35.3213 | | | |
| 16773 | 1080.48 | 27.7698 | DRGEpGPpGPA | Collagen alpha-1(I) | CO1A1_HUMAN (801-811) |
| | | | | chain | |
| 16779 | 1080.5 | 25.6942 | ApGDRGEpGPP | Collagen alpha-1(I) | CO1A1_HUMAN (798-808) |
| | | | | chain | |
| 16976 | 1084.43 | 25.2328 | | | |
| 17045 | 1085.42 | 20.7743 | | | |
| 17694 | 1096.48 | 26.0757 | ApGDRGEpGpP | Collagen alpha-1(I) | CO1A1_HUMAN (798-808) |
| | | | | chain | |
| 17830 | 1097.5 | 25.4051 | VIEHIMEDL | Protein S100-A9 | S10A9_HUMAN (58-66) |
| 17899 | 1098.52 | 20.8911 | | | |
| 17968 | 1099.49 | 28.2422 | DGGGSPKGDVDP | Sodium/potassium- | ATNG_HUMAN (7-18) |
| | | | | transporting ATPase | |
| | | | | subunit gamma | |
| 18029 | 1100.5 | 37.0367 | pGPPGpPGpPSP | Collagen alpha-6(IV) | CO4A6_HUMAN (432-443) |
| | | | | chain | |
| 18077 | 1101.47 | 22.2691 | | | |
| 18358 | 1107.43 | 25.1418 | | | |
| 18604 | 1110.39 | 33.6302 | | | |
| 18833 | 1113.46 | 27.2996 | | | |
| 18939 | 1114.48 | 24.2135 | | | |
| 18943 | 1114.49 | 25.5545 | SpGERGETGPp | Collagen alpha-1(III) | CO3A1_HUMAN (796-806) |
| | | | | chain | |
| 18988 | 1115.5 | 21.5669 | | | |
| 19046 | 1116.53 | 20.8514 | | | |
| 19137 | 1118.49 | 37.7593 | | | |
| 19284 | 1121.5 | 27.6767 | DGRpGPpGPpGA | Collagen alpha-1(I) | CO1A1_HUMAN (562-573) |
| | | | | chain | |
| 19655 | 1126.51 | 25.5171 | GGpGSDGKpGPpG | Collagen alpha-1(III) | CO3A1_HUMAN (543-555) |
| | | | | chain | |
| 19773 | 1128.39 | 33.592 | DFDDFNLED | CD99 antigen-like | C99L2_HUMAN (26-34) |
| | | | | protein 2 | |
| 19791 | 1128.49 | 25.6493 | | | |
| 19828 | 1129.46 | 27.9149 | | | |
| 19871 | 1130.34 | 35.393 | | | |
| 20072 | 1134.58 | 23.6558 | PIGQEGAPGRPG | Collagen alpha-2(IV) | CO4A2_HUMAN |
| | | | | chain | (1463-1474) |
| 20204 | 1137.51 | 26.4263 | DTNADKQLSF | Protein S100-A9 | S10A9_HUMAN (67-76) |
| 20457 | 1138.59 | 19.5068 | | | |
| 20659 | 1139.49 | 20.9651 | | | |
| 20700 | 1140.52 | 25.3846 | YNKYPDAVAT | Osteopontin | OSTP_HUMAN (33-42) |
| 20750 | 1141.52 | 24.5065 | | | |
| 20756 | 1141.54 | 37.3328 | GPpGPpGPPGPpA | Collagen alpha-1(VIII) | CO8A1_HUMAN (560-572) |
| | | | | chain | |
| 20862 | 1143.52 | 36.9674 | GLPGPpGPpGPpG | Collagen alpha-1 (I) | CO1A1_HUMAN (139-151) |
| | | | | chain | |
| 20896 | 1144.36 | 35.4858 | | | |
| 21641 | 1155.49 | 20.8473 | | | |
| 21747 | 1157.54 | 37.4441 | GPPGPpGppGPPS | Collagen alpha-1(I) | CO1A1_HUMAN |
| | | | | chain | (1181-1193) |
| 21972 | 1160.36 | 35.6006 | | | |
| 22089 | 1162.54 | 20.112 | | | |
| 22725 | 1171.51 | 29.1847 | | | |
| 22835 | 1173.53 | 37.4904 | GPpGPpGPpGPVT | Collagen alpha-1(XVII) | COHA1_HUMAN |
| | | | | chain | (1036-1048) |
| 23224 | 1178.39 | 20.7118 | | | |
| 23356 | 1179.52 | 37.4916 | | | |
| 23409 | 1180.52 | 35.6997 | | | |
| 23423 | 1180.54 | 25.6232 | | | |
| 23518 | 1182.55 | 28.273 | | | |
| 23628 | 1184.56 | 26.4035 | KpGEQGVpGDLG | Collagen alpha-1(I) | CO1A1_HUMAN (657-668) |
| | | | | chain | |
| 23697 | 1186.53 | 22.3938 | DDGEAGKpGRpG | Collagen alpha-1(I) | CO1A1_HUMAN (231-242) |
| | | | | chain | |
| 23724 | 1187.36 | 35.6892 | | | |
| 23968 | 1191.52 | 36.1767 | pPGSNGNpGPpGP | Collagen alpha-1(II) | CO2A1_HUMAN (907-919) |
| | | | | chain | |
| 24113 | 1194.55 | 29.2014 | | | |
| 24168 | 1195.48 | 37.5052 | | | |
| 24291 | 1196.33 | 36.1119 | | | |
| 24308 | 1196.52 | 21.0014 | | | |
| 24449 | 1199.58 | 21.9516 | | | |
| 24510 | 1200.54 | 24.1426 | | | |
| 24660 | 1203.57 | 26.7602 | YLGGSPKGDVDP | Sodium/potassium- | (5-16) |
| | | | | transporting ATPase | |
| | | | | subunit gamma | |
| | | | | isoform 2 | |
| 24958 | 1209.53 | 26.1548 | GPpGPDGNKGEpG | Collagen alpha-2(I) | CO1A2_HUMAN (613-625) |
| | | | | chain | |
| 25053 | 1211.54 | 25.8213 | GPpGEAGKpGEQG | Collagen alpha-1(I) | CO1A1_HUMAN (650-662) |
| | | | | chain | |
| 25099 | 1212.56 | 21.204 | | | |
| 25294 | 1215.43 | 27.5883 | | | |
| 25295 | 1215.45 | 26.8785 | | | |
| 25363 | 1216.54 | 24.2435 | | | |
| 25429 | 1217.53 | 35.7806 | | | |
| 25866 | 1223.55 | 27.457 | GPGGDKGDTGPpGP | Collagen alpha-1(III) | CO3A1_HUMAN (624-637) |
| | | | | chain | |
| 26098 | 1225.32 | 35.6881 | | | |
| 26163 | 1226.53 | 21.0162 | | | |
| 26412 | 1231.42 | 21.5127 | | | |
| 26670 | 1235.56 | 26.6462 | GQDGRpGPpGPpG | Collagen alpha-1(I) | CO1A1_HUMAN (560-572) |
| | | | | chain | |
| 26879 | 1238.56 | 21.1369 | | | |
| 27350 | 1247.52 | 22.0008 | DKGETGEQGDRG | Collagen alpha-1(I) | CO1A1_HUMAN |
| | | | | chain | (1095-1106) |
| 27742 | 1251.62 | 22.5253 | DGVPGKDGPRGPT | Collagen alpha-1(III) | CO3A1_HUMAN (752-764) |
| | | | | chain | |
| 28005 | 1255.48 | 35.7697 | | | |
| 28103 | 1257.44 | 33.9189 | | | |
| 28132 | 1257.64 | 19.9186 | | | |
| 28281 | 1260.56 | 21.8281 | | | |
| 28385 | 1262.47 | 38.233 | | | |
| 28466 | 1263.54 | 22.7286 | | | |
| 28561 | 1265.59 | 27.0867 | SpGPDGKTGPpGPA | Collagen alpha-1(I) | CO1A1_HUMAN (546-559) |
| | | | | chain | |
| 28669 | 1267.54 | 21.1725 | | | |
| 28747 | 1268.57 | 27.2482 | SpGERGETGPpGP | Collagen alpha-1(III) | CO3A1_HUMAN (796-808) |
| | | | | chain | |
| 28850 | 1270.55 | 29.3808 | | | |
| 29279 | 1276.4 | 35.9233 | | | |
| 29538 | 1281.59 | 27.0902 | | | |
| 29685 | 1283.55 | 27.2665 | | | |
| 29823 | 1286.42 | 36.407 | | | |
| 29840 | 1286.54 | 29.4051 | | | |
| 29906 | 1287.59 | 21.8737 | LGPHAGDVEGHLS | Apolipoprotein A-IV | APOA4_HUMAN (329-341) |
| 30136 | 1292.39 | 36.1593 | | | |
| 30524 | 1296.61 | 21.6926 | | | |
| 30575 | 1297.58 | 27.365 | SpGSpGPDGKTGPp | Collagen alpha-1(I) | CO1A1_HUMAN (543-556) |
| | | | | chain | |
| 30699 | 1299.58 | 22.3818 | | | |
| 31275 | 1310.59 | 21.3883 | | | |
| 31480 | 1312.55 | 29.7677 | | | |
| 31524 | 1312.62 | 20.0087 | | | |
| 32022 | 1319.58 | 20.8863 | | | |
| 32171 | 1321.59 | 28.371 | ApGDRGEpGPpGPA | Collagen alpha-1(I) | CO1A1_HUMAN (798-811) |
| | | | | chain | |
| 32336 | 1324.57 | 21.266 | | | |
| 32343 | 1324.59 | 28.7015 | TGPGGDKGDTGPpGP | Collagen alpha-1(III) | CO3A1_HUMAN (623-637) |
| | | | | chain | |
| 32470 | 1326.55 | 29.2043 | SpGGpGSDGKpGPpG | Collagen alpha-1(III) | CO3A1_HUMAN (541-555) |
| | | | | chain | |
| 32471 | 1326.56 | 27.1105 | | | |
| 32481 | 1326.57 | 21.6723 | | | |
| 32823 | 1332.54 | 21.7375 | | | |
| 32874 | 1333.42 | 36.108 | | | |
| 33135 | 1338.6 | 23.9878 | | | |
| 33209 | 1339.6 | 27.4859 | SpGERGETGPpGPA | Collagen alpha-1(III) | CO3A1_HUMAN (796-809) |
| | | | | chain | |
| 33238 | 1340.6 | 28.3984 | | | |
| 33812 | 1352.56 | 29.7655 | DSGSSEEQGGSSRA | Polymeric-immunoglobulin | PIGR_HUMAN (626-639) |
| | | | | receptor | |
| 33938 | 1353.59 | 21.483 | | | |
| 33973 | 1353.66 | 25.6316 | KGEAGLpGApGSPGQ | Collagen alpha-1(XIX) | COJA1_HUMAN (291-305) |
| | | | | chain | |
| 34009 | 1354.62 | 35.7097 | | | |
| 34017 | 1354.64 | 22.1393 | | | |
| 34267 | 1359.61 | 23.1947 | | | |
| 34339 | 1361.63 | 21.9874 | | | |
| 34432 | 1363.43 | 36.3375 | | | |
| 34766 | 1367.64 | 38.8826 | PpGPpGPpGPPGTPV | Collagen alpha-1(XVIII) | COIA1_HUMAN |
| | | | | chain | (1256-1270) |
| 35204 | 1375.61 | 23.245 | | | |
| 35339 | 1378.61 | 28.822 | ApGEDGRpGPpGPQ | Collagen alpha-1(II) | CO2A1_HUMAN (580-593) |
| | | | | chain | |
| 35424 | 1380.64 | 23.8305 | | | |
| 35550 | 1383.64 | 38.9402 | | | |
| 35979 | 1390.44 | 36.9459 | | | |
| 36500 | 1399.62 | 28.7444 | | | |
| 36566 | 1401.38 | 36.5567 | | | |
| 36586 | 1401.66 | 40.0387 | | | |
| 36759 | 1405.61 | 39.0354 | | | |
| 36769 | 1405.64 | 20.1391 | DGPpGRDGQpGHKG | Collagen alpha-2(I) | CO1A2_HUMAN (933-946) |
| | | | | chain | |
| 36868 | 1407.66 | 37.2292 | | | |
| 36988 | 1408.66 | 39.1338 | GPPGppGPpGPPGPPS | Collagen alpha-1(I) | CO1A1_HUMAN |
| | | | | chain | (1178-1193) |
| 37056 | 1409.58 | 22.0427 | | | |
| 37124 | 1410.62 | 19.897 | | | |
| 37340 | 1415.64 | 23.5532 | | | |
| 37603 | 1421.49 | 37.0163 | | | |
| 37610 | 1421.59 | 38.7131 | | | |
| 37662 | 1422.6 | 21.7229 | | | |
| 37903 | 1424.66 | 39.2996 | GLPGPpGPpGSFLSN | Collagen alpha-1(XVII) | COHA1_HUMAN (885-899) |
| | | | | chain | |
| 37949 | 1425.59 | 22.3178 | | | |
| 38011 | 1426.64 | 22.4206 | PpGKNGDDGEAGKpG | Collagen alpha-1(I) | CO1A1_HUMAN (225-239) |
| | | | | chain | |
| 38586 | 1435.51 | 19.7504 | | | |
| 38752 | 1438.45 | 36.7639 | | | |
| 38780 | 1438.66 | 30.2014 | | | |
| 38790 | 1438.66 | 22.0085 | | | |
| 38798 | 1438.67 | 27.8755 | GLpGTGGPpGENGKpG | Collagen alpha-1(III) | CO3A1_HUMAN (642-657) |
| | | | | chain | |
| 38879 | 1439.66 | 29.8171 | TIDEKGTEAAGAMF | Alpha-1-antitrypsin | A1AT_HUMAN (363-376) |
| 38910 | 1440.56 | 24.3004 | DEAGSEADHEGTHS | Fibrinogen alpha | FIBA_HUMAN (605-618) |
| | | | | chain | |
| 38951 | 1440.66 | 39.2845 | | | |
| 39064 | 1442.63 | 27.6328 | SpGENGApGQMGPRG | Collagen alpha-1(I) | CO1A1_HUMAN (291-305) |
| | | | | chain | |
| 39275 | 1445.62 | 37.359 | | | |
| 39322 | 1446.64 | 39.4279 | | | |
| 39607 | 1447.7 | 19.4724 | | | |
| 40091 | 1449.64 | 21.8561 | | | |
| 40294 | 1452.66 | 23.6065 | | | |
| 40487 | 1457.6 | 21.9348 | FHDDGFLAFPGHV | Basement membrane- | PGBM_HUMAN (4206-4218) |
| | | | | specific heparan | |
| | | | | sulfate proteoglycan | |
| | | | | core protein | |
| 40541 | 1458.63 | 27.9416 | SpGENGApGQmGPRG | Collagen alpha-1(I) | CO1A1_HUMAN (291-305) |
| | | | | chain | |
| 40737 | 1462.62 | 39.4214 | | | |
| 41434 | 1466.65 | 28.5188 | | | |
| 41471 | 1467.67 | 21.2878 | | | |
| 41601 | 1469.67 | 23.6936 | | | |
| 41665 | 1470.68 | 21.0804 | | | |
| 41972 | 1478.61 | 39.3027 | | | |
| 42188 | 1483.66 | 22.586 | GPpGKNGDDGEAGKpG | Collagen alpha-1(I) | CO1A1_HUMAN (224-239) |
| | | | | chain | |
| 42216 | 1483.7 | 20.6531 | | | |
| 42378 | 1486.68 | 21.1523 | | | |
| 42404 | 1487.65 | 29.6217 | GLSMDGGGSPKGDVDP | Sodium/potassium- | ATNG_HUMAN (3-18) |
| | | | | transporting ATPase | |
| | | | | subunit gamma | |
| 42594 | 1491.74 | 39.8339 | VGPpGpPGPPGPPGPPS | Collagen alpha-1(I) | CO1A1_HUMAN |
| | | | | chain | (1177-1193) |
| 42722 | 1493.63 | 21.8415 | | | |
| 42776 | 1494.66 | 30.3993 | EpGDAGAKGDAGPpGPA | Collagen alpha-1(I) | CO1A1_HUMAN (828-844) |
| | | | | chain | |
| 42866 | 1496.68 | 30.3745 | | | |
| 42867 | 1496.63 | 22.3363 | | | |
| 43226 | 1503.66 | 29.682 | GLSmDGGGSPKGDVDP | Sodium/potassium- | ATNG_HUMAN (3-18) |
| | | | | transporting ATPase | |
| | | | | subunit gamma | |
| 43442 | 1507.74 | 40.0239 | VGPpGPpGPpGPPGPPS | Collagen alpha-1(I) | CO1A1_HUMAN |
| | | | | chain | (1177-1193) |
| 43543 | 1508.68 | 29.3327 | GSpGSpGPDGKTGPPGp | Collagen alpha-1(I) | CO1A1_HUMAN (542-558) |
| | | | | chain | |
| 43605 | 1509.79 | 29.5021 | SVIDQSRVLNLGPI | Uromodulin | UROM_HUMAN (591-604) |
| 43828 | 1512.69 | 26.6212 | | | |
| 43851 | 1513.44 | 36.7892 | | | |
| 43863 | 1513.63 | 29.5129 | | | |
| 44146 | 1518.6 | 19.3715 | | | |
| 44464 | 1521.69 | 30.5282 | GDSDDDEPPPLPRL | Membrane associated | PGRC1_HUMAN (55-68) |
| | | | | progesterone receptor | |
| | | | | component 1 | |
| 44592 | 1523.67 | 21.9661 | | | |
| 44633 | 1523.84 | 29.7538 | VIDQSRVLNLGPIT | Uromodulin | UROM_HUMAN (592-605) |
| 44679 | 1524.65 | 20.0263 | | | |
| 44802 | 1526.69 | 23.9186 | | | |
| 45021 | 1532.62 | 26.3459 | | | |
| 45243 | 1535.68 | 22.0021 | VEHSDLSFSKDWS | Beta-2-microglobulin | B2MG_HUMAN (69-81) |
| 45980 | 1552.5 | 37.2147 | | | |
| 46091 | 1554.66 | 28.5929 | | | |
| 46184 | 1556.74 | 40.0323 | | | |
| 46338 | 1560.58 | 21.7691 | | | |
| 46369 | 1560.7 | 29.7854 | | | |
| 46542 | 1561.45 | 36.9566 | | | |
| 46606 | 1562.69 | 22.4615 | | | |
| 46725 | 1564.72 | 28.37 | | | |
| 46756 | 1565.69 | 26.3032 | | | |
| 47285 | 1575.75 | 30.1998 | IFPPSDEQLKSGTAS | Ig kappa chain C | IGKC_HUMAN (9-23) |
| | | | | region | |
| 47402 | 1576.67 | 43.3003 | | | |
| 47855 | 1576.74 | 19.5079 | YKRKANDESNEHS | Osteopontin | OSTP_HUMAN (246-258) |
| 48394 | 1583.7 | 23.2678 | NDGApGKNGERGGpGGp | Collagen alpha-1(III) | CO3A1_HUMAN (586-602) |
| | | | | chain | |
| 48417 | 1584.51 | 37.4959 | | | |
| 48520 | 1586.74 | 28.8819 | | | |
| 48580 | 1588.71 | 30.1503 | TGLSMDGGGSPKGDVDP | Sodium/potassium- | ATNG_HUMAN (2-18) |
| | | | | transporting ATPase | |
| | | | | subunit gamma | |
| 48751 | 1592.7 | 22.1803 | | | |
| 49122 | 1592.73 | 19.5192 | | | |
| 49243 | 1593.69 | 22.3763 | | | |
| 49295 | 1594.73 | 23.0529 | ApGGKGDAGApGERGPp | Collagen alpha-1(III) | CO3A1_HUMAN (670-687) |
| | | | G | chain | |
| 49334 | 1595.69 | 20.1503 | | | |
| 49608 | 1602.74 | 28.9718 | | | |
| 49713 | 1604.73 | 30.3377 | TGLSmDGGGSPKGDVDP | Sodium/potassium- | ATNG_HUMAN (2-18) |
| | | | | transporting ATPase | |
| | | | | subunit gamma | |
| 49948 | 1608.68 | 22.349 | | | |
| 49958 | 1608.73 | 30.9321 | SGDSDDDEPPPLPRL | Membrane associated | PGRC1_HUMAN (54-68) |
| | | | | progesterone receptor | |
| | | | | component 1 | |
| 50008 | 1609.75 | 30.2043 | TGSpGSpGPDGKTGPpGP | Collagen alpha-1(I) | CO1A1_HUMAN (541-558) |
| | | | | chain | |
| 50056 | 1610.87 | 30.2917 | SVIDQSRVLNLGPIT | Uromodulin | UROM_HUMAN (591-605) |
| 50172 | 1612.76 | 23.3804 | ApGSKGDTGAKGEpGPV | Collagen alpha-1(I) | CO1A1_HUMAN (438-455) |
| | | | G | chain | |
| 50212 | 1613.82 | 23.986 | VGGGEQPPPAPAPRRE | Xylosyltransferase 1 | XYLT1_HUMAN (51-66) |
| 50384 | 1616.73 | 40.25 | | | |
| 50638 | 1620.7 | 22.6628 | | | |
| 50766 | 1622.71 | 30.44 | | | |
| 50838 | 1623.73 | 29.8577 | | | |
| 50840 | 1623.73 | 24.1239 | DGApGKNGERGGpGGpG | Collagen alpha-1(III) | CO3A1_HUMAN (587-604) |
| | | | P | chain | |
| 50904 | 1624.55 | 37.7263 | | | |
| 51175 | 1630.74 | 20.6473 | EGSpGRDGSpGAKGDRG | Collagen alpha-1(I) | CO1A1_HUMAN |
| | | | | chain | (1021-1037) |
| 51328 | 1632.71 | 40.147 | | | |
| 51838 | 1635.65 | 37.3773 | | | |
| 51865 | 1635.76 | 30.3357 | FIFPPSDEQLKSGTA | Ig kappa chain C | IGKC_HUMAN (8-22) |
| | | | | region | |
| 51916 | 1636.7 | 20.0325 | | | |
| 52189 | 1640.58 | 23.2418 | | | |
| 52320 | 1643.74 | 29.5324 | | | |
| 52446 | 1645.76 | 20.6137 | | | |
| 52730 | 1649.71 | 19.5786 | | | |
| 52769 | 1649.73 | 22.643 | | | |
| 53328 | 1657.72 | 22.8973 | | | |
| 53375 | 1658.59 | 21.4575 | | | |
| 53393 | 1658.73 | 29.9416 | DAPGQYGAYFHDDGF | Basement membrane- | PGBM_HUMAN (4197-4211) |
| | | | | specific heparan | |
| | | | | sulfate proteoglycan | |
| | | | | core protein | |
| 53744 | 1666.78 | 30.6643 | KpGEQGVpGDLGApGPS | Collagen alpha-1(I) | CO1A1_HUMAN (657-674) |
| | | | G | chain | |
| 53800 | 1667.79 | 40.5608 | GPPGFTGPpGPpGPPGPP | Collagen alpha-1(IV) | CO4A1_HUMAN (197-215) |
| | | | G | chain | |
| 53866 | 1668.81 | 40.4656 | | | |
| 53957 | 1669.69 | 21.4647 | DEAGSEADHEGTHSTK | Fibrinogen alpha | FIBA_HUMAN (605-620) |
| | | | | chain | |
| 54184 | 1673.76 | 40.717 | | | |
| 54269 | 1675.71 | 29.2032 | GpPGPpGTSGHpGSpGSp | Collagen alpha-1(III) | CO3A1_HUMAN (180-198) |
| | | | G | chain | |
| 54421 | 1679.7 | 22.6046 | ppGDSGppGEKGDPGRP | Collagen alpha-1(VII) | CO7A1_HUMAN |
| | | | | chain | (1605-1621) |
| 54438 | 1679.95 | 23.8176 | VIDQSRVLNLGPITR | Uromodulin | UROM_HUMAN (592-606) |
| 54703 | 1684.71 | 29.6453 | | | |
| 55001 | 1689.8 | 27.5541 | | | |
| 55143 | 1692.8 | 30.8875 | PpGEAGKpGEQGVPGDL | Collagen alpha-1(I) | CO1A1_HUMAN (651-668) |
| | | | G | chain | |
| 55314 | 1693.76 | 20.5058 | | | |
| 55315 | 1693.76 | 23.4788 | PpGPPGkNGDDGEAGKp | Collagen alpha-1(I) | CO1A1_HUMAN (222-239) |
| | | | G | chain | |
| 55450 | 1694.78 | 23.5864 | GpAGpAGPRGSpGERGE | Collagen alpha-2(I) | CO1A2_HUMAN (700-717) |
| | | | V | chain | |
| 55484 | 1695.73 | 23.1763 | | | |
| 55516 | 1696.72 | 23.9499 | | | |
| 55582 | 1697.74 | 30.8785 | NGApGNDGAKGDAGApG | Collagen alpha-1(I) | CO1A1_HUMAN (700-719) |
| | | | ApG | chain | |
| 55917 | 1703.84 | 33.5849 | DHDVGSELPPEGVLGAL | ProSAAS | Q9UHG2_HUMAN (223-239) |
| 56011 | 1705.73 | 40.4367 | | | |
| 56099 | 1707.81 | 23.5945 | | | |
| 56139 | 1708.79 | 30.5905 | pPGEAGKpGEQGVpGDL | Collagen alpha-1(I) | CO1A1_HUMAN (651-668) |
| | | | G | chain | |
| 56493 | 1716.66 | 20.1773 | | | |
| 56514 | 1716.77 | 27.9978 | | | |
| 56662 | 1720.69 | 19.6657 | | | |
| 56806 | 1723.52 | 37.744 | | | |
| 56884 | 1725.59 | 38.322 | | | |
| 56992 | 1728.77 | 36.767 | | | |
| 57265 | 1732.77 | 28.1753 | | | |
| 57360 | 1734.66 | 19.8976 | | | |
| 57531 | 1737.78 | 31.0012 | TGSpGSpGPDGKTGPPGp | Collagen alpha-1(I) | CO1A1_HUMAN (541-560) |
| | | | AG | chain | |
| 57537 | 1737.78 | 23.7334 | NDGApGKNGERGGpGGp | Collagen alpha-1(III) | CO3A1_HUMAN (586-604) |
| | | | GP | chain | |
| 57888 | 1746.59 | 38.2127 | | | |
| 58050 | 1749.81 | 30.6146 | GPpGEAGKpGEQGVPGD | Collagen alpha-1(I) | CO1A1_HUMAN (650-668) |
| | | | LG | chain | |
| 58084 | 1750.78 | 23.8278 | GPpGPpGKNGDDGEAGK | Collagen alpha-1(I) | CO1A1_HUMAN (221-239) |
| | | | pG | chain | |
| 58355 | 1754.9 | 31.2589 | | | |
| 58633 | 1761.69 | 19.3667 | | | |
| 58759 | 1761.83 | 21.6467 | DGEAGKpGRPGERGPpG | Collagen alpha-1(I) | CO1A1_HUMAN (232-249) |
| | | | P | chain | |
| 58880 | 1764.68 | 19.9065 | | | |
| 58941 | 1765.81 | 31.0002 | GPpGEAGKpGEQGVpGD | Collagen alpha-1(I) | CO1A1_HUMAN (650-668) |
| | | | LG | chain | |
| 59142 | 1769.71 | 28.1352 | | | |
| 59149 | 1769.78 | 30.9769 | | | |
| 59295 | 1773.82 | 34.6048 | | | |
| 59368 | 1775.79 | 31.4772 | FGASAGTGDLSDNHDIIS | Vesicular integral- | LMAN2_HUMAN (253-270) |
| | | | | membrane protein | |
| | | | | VIP36 | |
| 59773 | 1783.79 | 39.8152 | | | |
| 59928 | 1788.84 | 29.8728 | | | |
| 60126 | 1793.88 | 32.3738 | EEAPSLRPAPPPISGGGY | Fibrinogen beta | FIBB_HUMAN (54-71) |
| | | | | chain | |
| 60149 | 1794.8 | 23.9153 | GNDGApGKNGERGGpGG | Collagen alpha-1(III) | CO3A1_HUMAN (585-604) |
| | | | pGP | chain | |
| 60248 | 1796.77 | 30.9215 | QGEAGQKGDAGAPGpQG | Collagen alpha-2(I) | CO1A2_HUMAN (851-870) |
| | | | pSG | chain | |
| 60352 | 1798.72 | 36.9482 | | | |
| 60628 | 1806.83 | 23.0614 | | | |
| 60751 | 1807.81 | 20.6486 | SVDETGQmSATAKGRVR | Retinol-binding | RET4_HUMAN (64-80) |
| | | | | protein 4 | |
| 60816 | 1808.79 | 23.7176 | | | |
| 60975 | 1812.79 | 24.1374 | | | |
| 61192 | 1816.9 | 33.7527 | | | |
| 61221 | 1817.69 | 20.2344 | | | |
| 61304 | 1818.83 | 30.9531 | GLpGTGGPpGENGKPGE | Collagen alpha-1(III) | CO3A1_HUMAN (642-661) |
| | | | PGp | chain | |
| 61332 | 1819.8 | 23.3631 | ApGAPGGKGDAGApGER | Collagen alpha-1(III) | CO3A1_HUMAN (667-687) |
| | | | GPpG | chain | |
| 61340 | 1819.83 | 32.1253 | | | |
| 61404 | 1821.83 | 31.7802 | | | |
| 61405 | 1821.82 | 30.1713 | GpPGEGRAGEpGTAGpT | Collagen alpha-2(VIII) | CO8A2_HUMAN (490-509) |
| | | | GpP | chain | |
| 61573 | 1825.79 | 20.135 | DEAGSEADHEGTHSTKR | Fibrinogen alpha | FIBA_HUMAN (605-621) |
| | | | | chain | |
| 61576 | 1825.8 | 31.931 | | | |
| 61711 | 1828.85 | 21.202 | SpGRDGSpGAKGDRGET | Collagen alpha-1(I) | CO1A1_HUMAN |
| | | | GP | chain | (1023-1041) |
| 61945 | 1834.83 | 31.095 | GLpGTGGPpGENGKpGEP | Collagen alpha-1(III) | CO3A1_HUMAN (642-661) |
| | | | Gp | chain | |
| 61984 | 1835.71 | 19.9131 | | | |
| 62000 | 1835.84 | 24.0379 | ApGApGGKGDAGApGER | Collagen alpha-1(III) | CO3A1_HUMAN (667-687) |
| | | | GPpG | chain | |
| 62080 | 1837.8 | 30.5569 | | | |
| 62256 | 1841.75 | 35.6598 | | | |
| 62323 | 1842.85 | 24.0672 | YSQGSKGPGEDFRMATL | Prostaglandin-H2 D- | PTGDS_HUMAN (132-148) |
| | | | | isomerase | |
| 62547 | 1847.89 | 43.6655 | | | |
| 62580 | 1848.81 | 30.8099 | | | |
| 62866 | 1854.82 | 40.9238 | | | |
| 63098 | 1858.84 | 24.2646 | | | |
| 63135 | 1859.8 | 31.3884 | | | |
| 63143 | 1859.83 | 24.4114 | NSGEpGApGSKGDTGAK | Collagen alpha-1(I) | CO1A1_HUMAN (432-451) |
| | | | GEp | chain | |
| 63209 | 1860.83 | 21.4001 | EGSpGRDGSpGAKGDRG | Collagen alpha-1(I) | CO1A1_HUMAN |
| | | | ET | chain | (1021-1039) |
| 63427 | 1865.81 | 32.982 | DAGPAGPKGEpGSpGEN | Collagen alpha-1(I) | CO1A1_HUMAN (279-299) |
| | | | GApG | chain | |
| 63517 | 1867.95 | 25.1463 | | | |
| 63812 | 1874.83 | 30.8238 | | | |
| 63910 | 1876.87 | 22.2013 | DDGEAGKPGRPGERGpP | Collagen alpha-1(I) | CO1A1_HUMAN (231-249) |
| | | | Gp | chain | |
| 64054 | 1878.79 | 20.7224 | | | |
| 64067 | 1878.85 | 32.1809 | | | |
| 64170 | 1880.9 | 43.9095 | | | |
| 64431 | 1885.65 | 38.82 | | | |
| 64493 | 1886.86 | 27.2961 | | | |
| 64869 | 1892.77 | 40.25 | | | |
| 64899 | 1892.97 | 24.5581 | | | |
| 65035 | 1895.07 | 21.396 | SVIDQSRVLNLGPITRK | Uromodulin | UROM_HUMAN (591-607) |
| 65257 | 1899.85 | 24.8347 | | | |
| 65312 | 1900.87 | 31.9971 | ETGPAGRpGEVGPpGPpG | Collagen alpha-1(I) | CO1A1_HUMAN (912-932) |
| | | | PAG | chain | |
| 65368 | 1901.82 | 43.8255 | | | |
| 65593 | 1907.77 | 23.7801 | | | |
| 66054 | 1915 | 33.6261 | | | |
| 66161 | 1916.77 | 20.3224 | | | |
| 66197 | 1916.95 | 34.193 | | | |
| 67217 | 1933.88 | 21.6245 | GDDGEAGKPGRpGERGP | Collagen alpha-1(I) | CO1A1_HUMAN (230-249) |
| | | | pGP | chain | |
| 67263 | 1934.79 | 19.9423 | | | |
| 67306 | 1935.81 | 20.6619 | | | |
| 67462 | 1938.88 | 21.3856 | kGNDGApGKNGERGGpG | Collagen alpha-1(III) | CO3A1_HUMAN (584-604) |
| | | | GpGP | chain | |
| 67612 | 1942.84 | 30.9624 | | | |
| 67632 | 1943.01 | 24.9416 | EAIPMSIPPEVKFNKPF | Alpha-1-antitrypsin | A1AT_HUMAN (378-394) |
| 67723 | 1945.88 | 41.9013 | | | |
| 67801 | 1947.88 | 31.6062 | | | |
| 67951 | 1950.85 | 35.7688 | | | |
| 68081 | 1954.01 | 32.4121 | | | |
| 68117 | 1954.97 | 25.3585 | SHTSDSDVPSGVTEVVVK | Clusterin | CLUS_HUMAN (391-409) |
| | | | L | | |
| 68411 | 1962.83 | 21.9701 | | | |
| 68663 | 1968.88 | 25.1998 | GEpGApGSKGDTGAKGEp | Collagen alpha-1(I) | CO1A1_HUMAN (434-455) |
| | | | GPVG | chain | |
| 69080 | 1976.88 | 32.3847 | | | |
| 69681 | 1989.88 | 32.4375 | SNGNpGPpGPSGSpGKD | Collagen alpha-1(III) | CO3A1_HUMAN (886-907) |
| | | | GPpGP | chain | |
| 69882 | 1993.88 | 32.1852 | | | |
| 69979 | 1996.79 | 20.9757 | | | |
| 70456 | 2008.9 | 32.2865 | | | |
| 70485 | 2009.88 | 32.2918 | | | |
| 70633 | 2013.89 | 31.7558 | AGpPGPPGppGTSGHpGS | Collagen alpha-1(III) | CO3A1_HUMAN (176-198) |
| | | | pGSpG | chain | |
| 70635 | 2013.91 | 25.1946 | NSGEpGApGSKGDTGAK | Collagen alpha-1(I) | CO1A1_HUMAN (432-453) |
| | | | GEpGP | chain | |
| 70803 | 2017.88 | 30.7718 | | | |
| 71171 | 2023.91 | 21.4813 | GEPGGkGERGApGEKGE | Collagen alpha-1(III) | CO3A1_HUMAN (819-840) |
| | | | GGpPG | chain | |
| 71312 | 2025.87 | 32.2255 | SEGSpGHpGQpGpPGPP | Collagen alpha-1(III) | CO3A1_HUMAN |
| | | | GApGp | chain | (1174-1195) |
| 71490 | 2029.85 | 20.3904 | | | |
| 71602 | 2030.93 | 32.6077 | PpGEAGKpGEQGVpGDL | Collagen alpha-1(I) | CO1A1_HUMAN (651-672) |
| | | | GAPGP | chain | |
| 71943 | 2034.88 | 19.5206 | | | |
| 72033 | 2034.94 | 25.4531 | | | |
| 72048 | 2035 | 40.1854 | | | |
| 72343 | 2042.07 | 25.1431 | EAIPMSIPPEVKFNKPFV | Alpha-1-antitrypsin | A1AT_HUMAN (378-395) |
| 72483 | 2045.86 | 34.1702 | | | |
| 72533 | 2046.92 | 32.5791 | PpGEAGKpGEQGVpGDL | Collagen alpha-1(I) | CO1A1_HUMAN (651-672) |
| | | | GApGP | chain | |
| 72641 | 2048.93 | 24.4632 | | | |
| 72896 | 2055.94 | 25.438 | SGEpGApGSKGDTGAKG | Collagen alpha-1(I) | CO1A1_HUMAN (433-455) |
| | | | EpGPVG | chain | |
| 73010 | 2058.94 | 23.1508 | | | |
| 73015 | 2059.01 | 33.0804 | ELTETGVEAAAASAISVAR | Plasma protease C1 | IC1_HUMAN (448-468) |
| | | | TL | inhibitor | |
| 73177 | 2062.93 | 26.579 | DAGApGAPGGKGDAGAp | Collagen alpha-1(III) | CO3A1_HUMAN (664-687) |
| | | | GERGPpG | chain | |
| 73291 | 2064.92 | 24.4599 | | | |
| 73434 | 2067.82 | 20.6208 | | | |
| 73913 | 2076.95 | 21.7789 | GPpGPpGKNGDDGEAGkp | Collagen alpha-1(I) | CO1A1_HUMAN (221-242) |
| | | | GRPG | chain | |
| 73989 | 2077.96 | 22.4848 | GDDGEAGKpGRpGERGP | Collagen alpha-1(I) | CO1A1_HUMAN (230-250) |
| | | | pGPQ | chain | |
| 74057 | 2079 | 24.6778 | | | |
| 74065 | 2078.93 | 26.6715 | DAGApGApGGKGDAGAp | Collagen alpha-1(III) | CO3A1_HUMAN (664-687) |
| | | | GERGPpG | chain | |
| 74187 | 2080.94 | 20.201 | DAHKSEVAHRFKDLGEEN | Serum albumin | ALBU_HUMAN (25-42) |
| 74273 | 2081.94 | 33.6612 | | | |
| 74420 | 2085.93 | 22.0672 | EGSpGRDGSpGAKGDRG | Collagen alpha-1(I) | CO1A1_HUMAN |
| | | | ETGPA | chain | (1021-1042) |
| 74703 | 2087.84 | 19.4213 | | | |
| 74987 | 2089.96 | 39.5167 | | | |
| 75248 | 2096.92 | 32.9972 | GApGNDGAKGDAGApGA | Collagen alpha-1(I) | CO1A1_HUMAN (701-725) |
| | | | pGSQGApG | chain | |
| 75846 | 2103.96 | 33.0368 | GPpGEAGKpGEQGVpGD | Collagen alpha-1(I) | CO1A1_HUMAN (650-672) |
| | | | LGApGP | chain | |
| 76053 | 2108.93 | 32.932 | | | |
| 76086 | 2109.92 | 24.069 | | | |
| 76090 | 2109.96 | 21.086 | | | |
| 76415 | 2117.93 | 32.9682 | SNGNpGpPGPSGSPGKD | Collagen alpha-1(III) | CO3A1_HUMAN (886-909) |
| | | | GPpGpAG | chain | |
| 76636 | 2124.84 | 20.3719 | | | |
| 76839 | 2128.98 | 26.9686 | DGKTGpPGPAGQDGRPG | Collagen alpha-1(I) | CO1A1_HUMAN (550-572) |
| | | | PpGppG | chain | |
| 77018 | 2133.96 | 27.7658 | DGQPGAKGEpGDAGAKG | Collagen alpha-1(I) | CO1A1_HUMAN (820-843) |
| | | | DAGPPGp | chain | |
| 77099 | 2135.96 | 25.8003 | GDAGApGApGGKGDAGA | Collagen alpha-1(III) | CO3A1_HUMAN (663-687) |
| | | | pGERGPpG | chain | |
| 77236 | 2138.95 | 24.235 | | | |
| 77684 | 2148.02 | 26.0398 | | | |
| 77763 | 2149.96 | 27.7553 | DGQpGAKGEpGDAGAKG | Collagen alpha-1(I) | CO1A1_HUMAN (820-843) |
| | | | DAGPPGp | chain | |
| 78073 | 2156.97 | 22.2173 | AEGSpGRDGSpGAKGDR | Collagen alpha-1(I) | CO1A1_HUMAN |
| | | | GETGPA | chain | (1020-1042) |
| 78792 | 2168.97 | 32.9057 | SDGQpGPpGPpGTAGFpG | Collagen alpha-1(III) | CO3A1_HUMAN (328-351) |
| | | | SpGAKG | chain | |
| 79136 | 2175.01 | 33.2806 | | | |
| 79720 | 2187.95 | 39.7827 | | | |
| 79786 | 2189 | 26.885 | ADGQPGAKGEPGDAGAK | Collagen alpha-1(I) | CO1A1_HUMAN (819-843) |
| | | | GDAGPpGP | chain | |
| 79918 | 2190.99 | 25.823 | | | |
| 80012 | 2191.99 | 22.3935 | NGDDGEAGkPGRpGERG | Collagen alpha-1(I) | CO1A1_HUMAN (229-250) |
| | | | PpGPQ | chain | |
| 80306 | 2194.97 | 20.1667 | NDGPpGRDGQpGHKGER | Collagen alpha-2(I) | CO1A2_HUMAN (932-952) |
| | | | GYpG | chain | |
| 80360 | 2196.02 | 33.1586 | | | |
| 80829 | 2203.64 | 35.2621 | | | |
| 81181 | 2210.76 | 37.0638 | | | |
| 81196 | 2210.95 | 33.6055 | NGApGNDGAKGDAGApG | Collagen alpha-1(I) | CO1A1_HUMAN (700-725) |
| | | | ApGSQGApG | chain | |
| 81272 | 2211.98 | 33.2336 | | | |
| 81457 | 2216.03 | 33.8306 | IGPpGPAGApGDKGESGP | Collagen alpha-1(I) | CO1A1_HUMAN (769-794) |
| | | | SGPAGPTG | chain | |
| 82015 | 2226.97 | 33.4565 | NGApGNDGAkGDAGApG | Collagen alpha-1(I) | CO1A1_HUMAN (700-725) |
| | | | ApGSQGApG | chain | |
| 82026 | 2226.99 | 26.2775 | GNSGEpGApGSKGDTGA | Collagen alpha-1(I) | CO1A1_HUMAN (431-455) |
| | | | KGEpGPVG | chain | |
| 82708 | 2235.05 | 34.1665 | GRTGDAGPVGPPGPpGp | Collagen alpha-1(I) | CO1A1_HUMAN |
| | | | pGpPGPPS | chain | (1169-1193) |
| 82784 | 2236.98 | 27.1389 | | | |
| 83081 | 2243.97 | 21.1604 | DGVSGGEGKGGSDGGG | CD99 antigen | CD99_HUMAN (97-120) |
| | | | SHRKEGEE | | |
| 83441 | 2248.97 | 33.6947 | | | |
| 84164 | 2257.87 | 35.9274 | | | |
| 84300 | 2261.03 | 27.1527 | LQGLpGTGGPpGENGKpG | Collagen alpha-1(II) | CO3A1_HUMAN (640-663) |
| | | | EpGPKG | chain | |
| 84484 | 2264.94 | 43.2938 | | | |
| 84704 | 2269.03 | 39.327 | | | |
| 84909 | 2274.04 | 33.5062 | | | |
| 85020 | 2276.02 | 27.2312 | ADGQPGAKGEpGDAGAK | Collagen alpha-1(I) | CO1A1_HUMAN (819-844) |
| | | | GDAGPpGPA | chain | |
| 85076 | 2277.01 | 27.2248 | | | |
| 85250 | 2280.94 | 36.2186 | | | |
| 85331 | 2282.02 | 22.2394 | ApGHpGPpGPVGPAGKS | Collagen alpha-1(III) | CO3A1_HUMAN |
| | | | GDRGESGP | chain | (1045-1069) |
| 85627 | 2289.04 | 33.5902 | | | |
| 85761 | 2292.02 | 27.2827 | ADGQpGAKGEpGDAGAK | Collagen alpha-1(I) | CO1A1_HUMAN (819-844) |
| | | | GDAGPpGPA | chain | |
| 86071 | 2298.99 | 27.0777 | | | |
| 86226 | 2302.9 | 43.1327 | | | |
| 86354 | 2305.06 | 34.9093 | | | |
| 86677 | 2308.02 | 27.3371 | ADGQpGAKGEpGDAGAK | Collagen alpha-1(I) | CO1A1_HUMAN (819-844) |
| | | | GDAGppGPA | chain | |
| 86951 | 2314.01 | 33.6555 | | | |
| 87365 | 2320.07 | 20.7338 | KNGDDGEAGKpGRpGER | Collagen alpha-1(I) | CO1A1_HUMAN (228-250) |
| | | | GPPGpQ | chain | |
| 87692 | 2327.91 | 21.0034 | | | |
| 88093 | 2336.04 | 26.6568 | | | |
| 88282 | 2339 | 34.0072 | GANGApGNDGAKGDAGA | Collagen alpha-1(I) | CO1A1_HUMAN (698-725) |
| | | | pGApGSQGApG | chain | |
| 88972 | 2349.04 | 27.3655 | GADGQPGAkGEpGDAGA | Collagen alpha-1(I) | CO1A1_HUMAN (818-844) |
| | | | KGDAGPpGPA | chain | |
| 89083 | 2352.05 | 26.7539 | | | |
| 89604 | 2360 | 33.9228 | | | |
| 89642 | 2361.11 | 20.7943 | GKNGDDGEAGKPGRpGE | Collagen alpha-1(I) | CO1A1_HUMAN (227-250) |
| | | | RGPpGPQ | chain | |
| 89900 | 2368.04 | 26.7503 | | | |
| 90344 | 2377.1 | 20.7997 | GKNGDDGEAGKpGRpGE | Collagen alaha-1(I) | CO1A1_HUMAN (227-250) |
| | | | RGPpGPQ | chain | |
| 90470 | 2380.08 | 36.5109 | | | |
| 90840 | 2389.24 | 22.3992 | MIEQNTKSPLFMGKVVNP | Alpha-1-antitrypsin | A1AT_HUMAN (398-418) |
| | | | TQK | | |
| 91342 | 2403.19 | 24.9373 | DDILASPPRLPEPQPYPG | Collagen alpha-1(XVIII) | COIA1_HUMAN |
| | | | APHH | chain | (1534-1555) |
| 91375 | 2404.02 | 20.2728 | | | |
| 91542 | 2407.09 | 27.6715 | LDGAKGDAGPAGPKGEp | Collagen alpha-1(I) | CO1A1_HUMAN (273-299) |
| | | | GSpGENGApG | chain | |
| 91669 | 2411.08 | 26.8029 | | | |
| 92029 | 2414.63 | 35.6167 | | | |
| 92231 | 2421 | 34.8608 | | | |
| 92410 | 2423.09 | 27.666 | LDGAKGDAGpAGPKGEp | Collagen alpha-1(I) | CO1A1_HUMAN (273-299) |
| | | | GSpGENGApG | chain | |
| 92841 | 2430.1 | 28.3281 | ADGQPGAKGEpGDAGAK | Collagen alpha-1(I) | CO1A1_HUMAN (819-846) |
| | | | GDAGPpGPAGP | chain | |
| 92871 | 2430.59 | 35.5423 | | | |
| 93227 | 2442.07 | 34.0967 | | | |
| 93361 | 2445.1 | 28.2378 | mASDASHALEAALEQMD | Liprin-beta-2 | LIPB2_HUMAN (1-24) |
| | | | GIIAGTK | | |
| 93417 | 2446.09 | 28.3726 | ADGQpGAKGEpGDAGAK | Collagen alpha-1(I) | CO1A1_HUMAN (819-846) |
| | | | GDAGpPGPAGP | chain | |
| 95084 | 2490.23 | 24.6804 | | | |
| 96716 | 2525.2 | 27.7364 | LRGGAGPpGPEGGKGAA | Collagen alpha-1(III) | CO3A1_HUMAN (694-723) |
| | | | GPpGPpGAAGTpG | chain | |
| 96879 | 2529.17 | 26.992 | | | |
| 97349 | 2541.19 | 27.8949 | KNGETGPQGPPGPTGPG | Collagen alpha-1(III) | CO3A1_HUMAN (610-637) |
| | | | GDKGDTGPpGP | chain | |
| 97463 | 2544.13 | 28.2599 | | | |
| 97506 | 2545.12 | 28.2016 | GPpGADGQpGAKGEpGD | Collagen alpha-1(I) | CO1A1_HUMAN (815-843) |
| | | | AGAKGDAGpPGP | chain | |
| 97599 | 2547.99 | 21.4417 | | | |
| 97736 | 2551.15 | 34.7151 | | | |
| 97965 | 2557.17 | 28.2473 | KNGETGPQGPpGPTGPG | Collagen alpha-1(III) | CO3A1_HUMAN (610-637) |
| | | | GDKGDTGPPGp | chain | |
| 98089 | 2559.18 | 19.4074 | | | |
| 98485 | 2560.12 | 34.1541 | | | |
| 98596 | 2563.15 | 21.2101 | | | |
| 98720 | 2565.14 | 23.7401 | | | |
| 98891 | 2567.13 | 34.802 | | | |
| 99021 | 2570.19 | 42.5602 | | | |
| 99251 | 2574.01 | 32.8084 | | | |
| 99475 | 2577.25 | 24.6659 | DDILASPPRLPEPQPYPG | Collagen alpha-1(XVIII) | COIA1_HUMAN |
| | | | APHHSS | chain | (1534-1557) |
| 99691 | 2582.17 | 23.67 | PEAEAEAEAGAGGEAAAE | Zinc finger protein | ZN653_HUMAN (8-35) |
| | | | EGAAGRKARG | 653 | |
| 99808 | 2584.23 | 35.1828 | LTGPIGPPGpAGApGDKG | Collagen alpha-1(I) | CO1A1_HUMAN (765-794) |
| | | | ESGPSGPAGPTG | chain | |
| 99919 | 2587.2 | 21.1 | | | |
| 100020 | 2589.06 | 22.5622 | | | |
| 100344 | 2599.19 | 28.2751 | AGPpGApGApGApGPVGP | Collagen alpha-1(I) | CO1A1_HUMAN |
| | | | AGKSGDRGETGP | chain | (1042-1071) |
| 101157 | 2616.16 | 28.3947 | GPpGADGQpGAKGEpGD | Collagen alpha-1(I) | CO1A1_HUMAN (815-844) |
| | | | AGAKGDAGpPGPA | chain | |
| 101786 | 2627.23 | 34.9933 | | | |
| 101839 | 2628.22 | 34.9664 | | | |
| 102269 | 2638.18 | 28.4204 | | | |
| 102371 | 2639.29 | 21.4189 | KEGGKGPRGETGPAGRp | Collagen alpha-1(I) | CO1A1_HUMAN (903-930) |
| | | | GEVGpPGPpGP | chain | |
| 102725 | 2642.21 | 27.696 | QGPpGPSGEEGKRGPNG | Collagen alpha-2(I) | CO1A2_HUMAN (369-397) |
| | | | EAGSAGPpGPpG | chain | |
| 102819 | 2644.22 | 21.1526 | GPpGKNGDDGEAGKPGR | Collagen alpha-1(I) | CO1A1_HUMAN (224-250) |
| | | | pGERGPpGpQ | chain | |
| 103022 | 2649.2 | 34.8471 | | | |
| 103198 | 2654.19 | 23.9249 | | | |
| 103224 | 2655.14 | 22.3309 | | | |
| 103493 | 2658.22 | 19.5022 | | | |
| 104195 | 2663.2 | 23.5069 | | | |
| 104786 | 2679.2 | 23.5289 | NRGERGSEGSPGHpGQp | Collagen alpha-1(III) | CO3A1_HUMAN |
| | | | GppGpPGAPGP | chain | (1168-1195) |
| 104954 | 2682.14 | 22.4918 | | | |
| 105105 | 2687.22 | 28.9855 | KDGEAGAQGPpGPAGPA | Collagen alpha-1(I) | CO1A1_HUMAN (612-641) |
| | | | GERGEQGPAGSpG | chain | |
| 105352 | 2695.2 | 23.5225 | NRGERGSEGSpGHpGQp | Collagen alpha-1(III) | CO3A1_HUMAN |
| | | | GppGPPGAPGp | chain | (1168-1195) |
| 105661 | 2702.21 | 38.0799 | | | |
| 106067 | 2713.23 | 29.2227 | PpGADGQpGAKGEpGDA | Collagen alpha-1(I) | CO1A1_HUMAN (816-846) |
| | | | GAKGDAGPpGPAGP | chain | |
| 106195 | 2716.37 | 20.1907 | LLKNGERIEKVEHSDLSFS | Beta-2-microglobulin | B2MG_HUMAN (59-81) |
| | | | KDWS | | |
| 106678 | 2726.25 | 28.9168 | KNGETGPQGPPGPTGPG | Collagen alpha-1(III) | CO3A1_HUMAN (610-639) |
| | | | GDKGDTGPpGPQG | chain | |
| 107198 | 2736.4 | 21.2973 | FFLPDEGKLQHLENELTH | Alpha-1-antitrypsin | A1AT_HUMAN (276-298) |
| | | | DIITK | | |
| 107460 | 2742.25 | 28.9803 | KNGETGPQGPPGPTGPG | Collagen alpha-1(III) | CO3A1_HUMAN (610-639) |
| | | | GDKGDTGPpGpQG | chain | |
| 107858 | 2751.34 | 29.2308 | | | |
| 108021 | 2754.27 | 29.6794 | GPpGADGQPGAKGEpGD | Collagen alpha-1(I) | CO1A1_HUMAN (815-846) |
| | | | AGAKGDAGpPGPAGP | chain | |
| 108574 | 2764.21 | 42.6266 | | | |
| 108724 | 2767.32 | 21.6729 | KEGGKGPRGETGPAGRp | Collagen alpha-1(I) | CO1A1_HUMAN (903-932) |
| | | | GEVGpPGPpGPAG | chain | |
| 110052 | 2799.09 | 25.0694 | | | |
| 110657 | 2816.33 | 28.3966 | | | |
| 110913 | 2823.33 | 29.1158 | LRGGAGpPGPEGGKGAA | Collagen alpha-1(III) | CO3A1_HUMAN (694-726) |
| | | | GpPGppGAAGTPGLQG | chain | |
| 111304 | 2834.19 | 22.4673 | | | |
| 111426 | 2837.36 | 23.8684 | IPVKQADSGSSEEKQLYN | Osteopontin | OSTP_HUMAN (17-42) |
| | | | KYPDAVAT | | |
| 111564 | 2841.26 | 24.5354 | ERGEAGIpGVpGAkGEDG | Collagen alpha-1(III) | CO3A1_HUMAN (448-477) |
| | | | KDGSpGEpGANG | chain | |
| 112106 | 2854.36 | 34.8636 | | | |
| 112515 | 2864.25 | 20.1472 | GRDGNpGNDGPpGRDGQ | Collagen alpha-2(I) | CO1A2_HUMAN (925-952) |
| | | | pGHKGERGYpG | chain | |
| 113439 | 2889.38 | 28.6515 | | | |
| 113452 | 2889.35 | 24.0847 | | | |
| 113765 | 2898.31 | 29.2496 | | | |
| 114086 | 2907.35 | 35.9551 | TGEVGAVGPpGFAGEKG | Collagen alpha-2(I) | CO1A2_HUMAN (831-863) |
| | | | PSGEAGTAGPpGTpGP | chain | |
| 114438 | 2917.37 | 29.038 | | | |
| 114702 | 2923.43 | 36.9151 | | | |
| 114823 | 2926.3 | 22.2183 | ESGREGAPGAEGSpGRD | Collagen alpha-1(I) | CO1A1_HUMAN |
| | | | GSpGAKGDRGETGP | chain | (1011-1041) |
| 115050 | 2932.32 | 34.1472 | | | |
| 115312 | 2939.15 | 33.7671 | | | |
| 116812 | 2977.18 | 19.5232 | | | |
| 117009 | 2977.37 | 29.118 | | | |
| 117317 | 2987.35 | 38.5457 | | | |
| 117524 | 2994.38 | 29.6026 | | | |
| 117823 | 3002.24 | 23.8009 | | | |
| 118053 | 3007.41 | 20.9513 | | | |
| 118163 | 3011.39 | 29.7495 | LTGSpGSpGpDGKTGPPG | Collagen alpha-1(I) | CO1A1_HUMAN (540-572) |
| | | | PAGQDGRPGPpGppG | chain | |
| 118224 | 3013.29 | 22.2946 | ESGREGApGAEGSpGRD | Collagen alpha-1(I) | CO1A1_HUMAN |
| | | | GSpGAKGDRGETGPA | chain | (1011-1042) |
| 118597 | 3021.35 | 23.4158 | DGVSGGEGKGGSDGGG | CD99 antigen | CD99_HUMAN (97-129) |
| | | | SHRKEGEEADAPGVIPG | | |
| 119292 | 3035.19 | 42.0209 | | | |
| 119660 | 3044.42 | 34.2873 | | | |
| 120192 | 3058.38 | 24.8236 | QNGEPGGKGERGApGEK | Collagen alpha-1(III) | CO3A1_HUMAN (817-850) |
| | | | GEGGppGVAGPpGGSGP | chain | |
| 120423 | 3064.32 | 20.5742 | EAGRDGNpGNDGPpGRD | Collagen alpha-2(I) | CO1A2_HUMAN (923-952) |
| | | | GQpGHkGERGYPG | chain | |
| 121070 | 3076.23 | 19.5773 | | | |
| 121413 | 3080.41 | 29.9804 | | | |
| 121716 | 3091.44 | 28.3964 | | | |
| 121775 | 3092.46 | 31.2493 | ADGQPGAkGEPGDAGAK | Collagen alpha-1(I) | CO1A1_HUMAN (819-854) |
| | | | GDAGPPGPAGpAGpPGPI | chain | |
| | | | G | | |
| 121940 | 3098.44 | 30.0554 | LTGNpGVQGPEGKLGPLG | Collagen alpha-2(V) | CO5A2_HUMAN (574-606) |
| | | | ApGEDGRpGpPGSIG | chain | |
| 121989 | 3100.42 | 29.9643 | | | |
| 122216 | 3106.19 | 19.5811 | | | |
| 122400 | 3108.45 | 31.284 | ADGQpGAKGEpGDAGAK | Collagen alpha-1(I) | CO1A1_HUMAN (819-854) |
| | | | GDAGpPGPAGPAGPPGpI | chain | |
| | | | G | | |
| 122623 | 3114.43 | 30.2902 | | | |
| 123106 | 3130.43 | 30.8219 | | | |
| 123287 | 3137.41 | 30.3462 | | | |
| 123634 | 3148.28 | 24.1552 | | | |
| 123750 | 3152.34 | 24.5511 | | | |
| 123969 | 3158.44 | 29.7137 | GERGSpGGpGAAGFpGA | Collagen alpha-1(III) | CO3A1_HUMAN (861-895) |
| | | | RGLpGpPGSNGNPGPpGp | chain | |
| 124193 | 3166.27 | 22.0589 | | | |
| 124735 | 3187.36 | 35.6177 | | | |
| 124886 | 3193.38 | 22.6436 | PpGESGREGAPGAEGSp | Collagen alpha-1(I) | CO1A1_HUMAN |
| | | | GRDGSpGAKGDRGETGP | chain | (1008-1041) |
| 124909 | 3194.42 | 30.3977 | | | |
| 125103 | 3202.43 | 30.6017 | SSQGGSLPSEEKGHPQE | Secretogranin-1 | SCG1_HUMAN (293-323) |
| | | | ESEESNVSMASLGE | | |
| 125263 | 3205.27 | 19.6576 | | | |
| 125749 | 3223.31 | 21.6127 | | | |
| 125799 | 3223.42 | 39.1265 | | | |
| 126699 | 3248.53 | 30.6604 | RTGEVGAVGpPGFAGEK | Collagen alpha-2(I) | CO1A2_HUMAN (830-865) |
| | | | GPSGEAGTAGPPGTpGp | chain | |
| | | | QG | | |
| 126982 | 3256.53 | 33.0342 | | | |
| 127106 | 3260.49 | 41.6176 | | | |
| 127237 | 3261.5 | 22.1948 | | | |
| 127354 | 3264.53 | 30.6582 | RTGEVGAVGpPGFAGEkG | Collagen alpha-2(I) | CO1A2_HUMAN (830-865) |
| | | | PSGEAGTAGPPGTpGpQ | chain | |
| | | | G | | |
| 127443 | 3266.48 | 30.0713 | | | |
| 127575 | 3271.49 | 30.7045 | NTGApGSpGVSGpKGDA | Collagen alpha-1(III) | CO3A1_HUMAN (910-946) |
| | | | GQpGEKGSPGAQGPPGA | chain | |
| | | | PGp | | |
| 127731 | 3276.24 | 33.377 | | | |
| 127848 | 3280.46 | 22.7334 | pPGESGREGApGAEGSp | Collagen alpha-1(I) | CO1A1_HUMAN |
| | | | GRDGSpGAKGDRGETGP | chain | (1008-1042) |
| | | | A | | |
| 127852 | 3280.56 | 25.8155 | AAGEPGKAGERGVpGpp | Collagen alpha-1(I) | CO1A1_HUMAN (588-624) |
| | | | GAVGPAGKDGEAGAQGP | chain | |
| | | | pGP | | |
| 128086 | 3286.56 | 30.9207 | | | |
| 128435 | 3295.53 | 25.4528 | DRGETGPAGPpGApGAP | Collagen alpha-1(I) | CO1A1_HUMAN |
| | | | GAPGPVGpAGKSGDRGE | chain | (1035-1071) |
| | | | TGP | | |
| 128525 | 3298.46 | 36.0541 | | | |
| 128650 | 3302.59 | 23.2932 | | | |
| 128936 | 3311.48 | 24.331 | SPGEAGRPGEAGLpGAK | Collagen alpha-1(I) | CO1A1_HUMAN (522-558) |
| | | | GLTGSPGSPGpDGkTGPP | chain | |
| | | | GP | | |
| 129019 | 3314.43 | 20.1405 | | | |
| 129131 | 3318.55 | 30.992 | | | |
| 129182 | 3320.51 | 24.2533 | | | |
| 129821 | 3333.36 | 19.4182 | | | |
| 130044 | 3336.51 | 39.3802 | | | |
| 130077 | 3337.45 | 22.8134 | GPpGESGREGApGAEGS | Collagen alpha-1(I) | CO1A1_HUMAN |
| | | | pGRDGSpGAKGDRGETG | chain | (1007-1042) |
| | | | PA | | |
| 130432 | 3349.42 | 35.8938 | | | |
| 130482 | 3350.55 | 31.0174 | | | |
| 130747 | 3359.58 | 31.8979 | PpGADGQPGAKGEpGDA | Collagen alpha-1(I) | CO1A1_HUMAN (816-854) |
| | | | GAKGDAGPpGPAGPAGP | chain | |
| | | | pGPIG | | |
| 130803 | 3361.56 | 24.2435 | | | |
| 131294 | 3375.57 | 31.9169 | | | |
| 131548 | 3384.58 | 30.8333 | | | |
| 131589 | 3385.55 | 25.4887 | | | |
| 131932 | 3399.35 | 42.1045 | | | |
| 132014 | 3400.58 | 31.185 | GPpGADGQPGAKGEpGD | Collagen alpha-1(I) | CO1A1_HUMAN (815-854) |
| | | | AGAKGDAGpPGPAGPAG | chain | |
| | | | PPGPIG | | |
| 132057 | 3401.66 | 23.4928 | | | |
| 133168 | 3432.59 | 32.0468 | | | |
| 133345 | 3439.6 | 39.225 | | | |
| 134053 | 3462.35 | 19.3741 | | | |
| 134635 | 3478.43 | 41.7414 | | | |
| 135412 | 3510.6 | 40.2442 | | | |
| 135676 | 3520.61 | 30.7976 | | | |
| 136129 | 3536.46 | 20.2362 | | | |
| 136403 | 3546.67 | 26.2234 | | | |
| 136697 | 3556.59 | 23.9547 | FAEEKAVADTRDQADGS | Polymeric- | PIGR_HUMAN (605-639) |
| | | | RASVDSGSSEEQGGSSR | immunoglobulin | |
| | | | A | receptor | |
| 136698 | 3556.6 | 22.636 | EDPQGDAAQKTDTSHHD | Alpha-1-antitrypsin | A1AT_HUMAN (25-56) |
| | | | QDHPTFNKITPNLAE | | |
| 136789 | 3559.71 | 26.5191 | | | |
| 136790 | 3559.71 | 24.9213 | | | |
| 137489 | 3582.7 | 19.465 | | | |
| 137922 | 3583.64 | 41.4711 | | | |
| 138036 | 3589.68 | 25.0338 | | | |
| 138067 | 3590.7 | 29.0044 | | | |
| 138143 | 3593.47 | 20.1962 | | | |
| 138813 | 3605.57 | 21.1852 | | | |
| 139064 | 3616.72 | 33.188 | DQGPVGRTGEVGAVGPp | Collagen alpha-2(I) | CO1A2_HUMAN (824-863) |
| | | | GFAGEKGPSGEAGTAGp | chain | |
| | | | PGTpGP | | |
| 139455 | 3630.44 | 21.7771 | | | |
| 139554 | 3632.74 | 33.2296 | | | |
| 140112 | 3657.67 | 40.7109 | | | |
| 140570 | 3677.77 | 24.4986 | | | |
| 140672 | 3681.72 | 32.0178 | | | |
| 140780 | 3685.83 | 22.1964 | GRPEAQPPPLSSEHKEPV | Neurosecretory | VGF_HUMAN (26-62) |
| | | | AGDAVPGPKDGSAPEVR | protein | |
| | | | GA | VGF | |
| 141007 | 3696.76 | 26.9428 | | | |
| 141019 | 3697.56 | 23.7039 | | | |
| 141109 | 3701.74 | 32.4441 | | | |
| 141672 | 3718.72 | 32.4816 | | | |
| 142080 | 3734.72 | 32.4972 | | | |
| 142141 | 3737.66 | 37.205 | | | |
| 143120 | 3765.6 | 20.1729 | | | |
| 143333 | 3774.72 | 22.9371 | | | |
| 143379 | 3775.75 | 25.5852 | | | |
| 143652 | 3788.82 | 25.1861 | | | |
| 143947 | 3801.77 | 33.4628 | DQGPVGRTGEVGAVGPp | Collagen alpha-2(I) | CO1A2_HUMAN (824-865) |
| | | | GFAGEKGPSGEAGTAGP | chain | |
| | | | pGTpGPQG | | |
| 144344 | 3817.79 | 33.5359 | | | |
| 144635 | 3831.81 | 28.4845 | | | |
| 145238 | 3858.84 | 25.8539 | | | |
| 145479 | 3871.79 | 27.5695 | | | |
| 146624 | 3927.82 | 33.5971 | | | |
| 146936 | 3943.83 | 33.6285 | | | |
| 146964 | 3944.71 | 24.5544 | | | |
| 147000 | 3945.91 | 22.0304 | | | |
| 147376 | 3959.69 | 19.9025 | | | |
| 147541 | 3968.6 | 21.0938 | | | |
| 148086 | 3986.65 | 20.6016 | | | |
| 148557 | 4002.62 | 20.6566 | | | |
| 148717 | 4008.81 | 23.4219 | | | |
| 149142 | 4025.6 | 20.784 | | | |
| 149624 | 4043.64 | 20.3849 | | | |
| 149760 | 4044.92 | 26.3671 | | | |
| 150139 | 4059.65 | 20.4488 | | | |
| 150441 | 4062.8 | 33.1373 | | | |
| 150781 | 4078.81 | 33.1371 | | | |
| 151244 | 4097.87 | 24.6106 | | | |
| 152744 | 4160.92 | 25.8191 | | | |
| 152967 | 4169.93 | 33.5832 | ERGEQGPAGSpGFQGLp | Collagen alpha-1(I) | CO1A1_HUMAN (630-674) |
| | | | GpAGppGEAGKpGEQGVP | chain | |
| | | | GDLGAPGPSG | | |
| 153832 | 4196.75 | 20.8379 | | | |
| 155132 | 4251.98 | 28.7652 | | | |
| 156081 | 4289.93 | 28.7838 | ARGNDGARGSDGQpGpp | Collagen alpha-1(III) | CO3A1_HUMAN (319-366) |
| | | | GPPGTAGFPGSpGAKGE | chain | |
| | | | VGpAGSpGSNGApG | | |
| 156445 | 4305.94 | 28.8296 | ARGNDGARGSDGQpGpP | Collagen alpha-1(III) | CO3A1_HUMAN (319-366) |
| | | | GPpGTAGFpGSpGAKGEV | chain | |
| | | | GpAGSpGSNGApG | | |
| 156878 | 4321.94 | 25.1991 | LQGLpGTGGppGENGKpG | Collagen alpha-1(III) | CO3A1_HUMAN (640-687) |
| | | | EpGpKGDAGAPGAPGGK | chain | |
| | | | GDAGAPGERGppG | | |
| 157882 | 4352.86 | 20.1681 | | | |
| 158341 | 4368.9 | 20.2129 | | | |
| 159259 | 4404.84 | 20.6659 | | | |
| 159396 | 4409.89 | 20.001 | | | |
| 160089 | 4436.08 | 26.3177 | | | |
| 167786 | 4771.07 | 20.1987 | | | |
| 173077 | 5213.09 | 22.4341 | | | |
| 174387 | 5427.17 | 22.935 | | | |
| 174987 | 5511.28 | 22.4639 | | | |
| 175094 | 5528.35 | 28.0044 | | | |
| 175343 | 5574.25 | 23.2009 | | | |
| 177848 | 6211.74 | 20.2851 | | | |
| 177971 | 6236.91 | 21.066 | | | |
| 178819 | 6541.76 | 20.6784 | | | |
| 179035 | 6650.64 | 25.5371 | | | |
| 181018 | 7906.95 | 19.5282 | | | |
| 181250 | 7958.47 | 34.3896 | | | |
| 184099 | 8853.77 | 21.097 | | | |
| 184206 | 8917.25 | 22.5451 | | | |
| 186609 | 10999.9 | 21.3719 | | | |
| 188895 | 11967.6 | 20.4687 | | | |
| 190524 | 14109.5 | 21.9308 | | | |

The evaluation of the polypeptides measured can be done on the basis of the presence or absence and/or amplitude of the markers taking the following limits into account:

TABLE 2

| | | | | log mean | | log mean | | |
|---|---|---|---|---|---|---|---|---|
| | | | | amp. | | Amp. | | |
| | Mass | CE Time | Frequency | (median) | Frequency | (median) | Wilcox | |
| ID | [DA] | [min] | HI | HI | control | Control | p-value | AUC |
| 912 | 826.201 | 33.4119 | 0.16 | 1.69 (1.53) | 0.35 | 1.88 (1.89) | 0.0014626820 | 0.6017219 |
| 1495 | 838.401 | 35.0611 | 0.16 | 1.05 (0.92) | 0.36 | 2.37 (2.43) | 0.0000578108 | 0.6294643 |
| 1577 | 840.407 | 23.1656 | 0.15 | 1.88 (1.93) | 0.5 | 2.02 (1.94) | 0.0000002388 | 0.6786777 |
| 2505 | 858.393 | 23.2367 | 0.34 | 1.86 (1.76) | 0.76 | 2.32 (2.31) | 0.0000000000 | 0.7605761 |
| 2659 | 860.362 | 26.1404 | 0.26 | 1.94 (1.93) | 0.71 | 2.50 (2.59) | 0.0000000000 | 0.7713648 |
| 3052 | 868.409 | 23.312 | 0.4 | 1.76 (1.74) | 0.57 | 2.11 (2.09) | 0.0004526336 | 0.6354698 |
| 3472 | 875.477 | 21.9392 | 0.15 | 1.81 (1.92) | 0.33 | 1.86 (1.96) | 0.0036423950 | 0.5908801 |
| 3608 | 879.499 | 19.881 | 0.23 | 1.83 (1.72) | 0.36 | 2.04 (2.09) | 0.0179793000 | 0.5796662 |
| 3743 | 883.41 | 23.2563 | 0.21 | 1.84 (1.93) | 0.47 | 1.97 (2.01) | 0.0000979105 | 0.6367453 |
| 3796 | 884.292 | 43.8066 | 0.29 | 1.67 (1.58) | 0.41 | 2.01 (1.92) | 0.0232707000 | 0.5800383 |
| 4730 | 898.315 | 26.0488 | 0.17 | 2.13 (2.20) | 0.36 | 2.16 (2.15) | 0.0030872610 | 0.5959290 |
| 4845 | 900.269 | 43.6572 | 0.28 | 2.25 (2.11) | 0.42 | 2.42 (2.32) | 0.0211585300 | 0.5813138 |
| 4976 | 902.413 | 20.8458 | 0.76 | 2.52 (2.56) | 0.9 | 2.67 (2.74) | 0.0005812378 | 0.6426446 |
| 5184 | 906.179 | 34.2619 | 0.33 | 1.88 (1.83) | 0.14 | 2.24 (2.30) | 0.0065992270 | 0.5835459 |
| 5661 | 911.265 | 34.3452 | 0.89 | 2.61 (2.65) | 0.89 | 3.00 (3.20) | 0.0005765453 | 0.6430166 |
| 5675 | 911.435 | 25.8763 | 0.29 | 2.15 (2.26) | 0.85 | 2.59 (2.58) | 0.0000000000 | 0.8384354 |
| 6803 | 928.372 | 35.4637 | 0.29 | 1.53 (1.61) | 0.44 | 1.94 (1.91) | 0.0024665110 | 0.6082058 |
| 7408 | 935.447 | 23.6811 | 0.05 | 1.51 (1.54) | 0.46 | 2.02 (1.95) | 0.0000000000 | 0.7118410 |
| 7953 | 940.438 | 25.1411 | 0.35 | 1.90 (1.83) | 0.15 | 1.71 (1.65) | 0.0005914545 | 0.6082058 |
| 8390 | 945.416 | 25.7221 | 0.34 | 1.97 (1.96) | 0.51 | 2.12 (2.18) | 0.0048398870 | 0.6052827 |
| 8503 | 947.473 | 24.741 | 0.19 | 1.85 (1.77) | 0.48 | 2.35 (2.34) | 0.0000009481 | 0.6712372 |
| 8695 | 949.219 | 34.3333 | 0.46 | 2.65 (2.71) | 0.38 | 2.81 (2.81) | 0.0260201500 | 0.5829082 |
| 8725 | 949.404 | 25.7932 | 0.04 | 1.95 (1.86) | 0.17 | 2.21 (2.27) | 0.0260007250 | 0.5821642 |
| 9930 | 963.491 | 21.8073 | 0.34 | 2.04 (2.09) | 0.48 | 2.15 (2.16) | 0.0260012500 | 0.5823767 |
| 11282 | 980.5 | 22.4055 | 0.26 | 1.75 (1.76) | 0.48 | 2.00 (2.05) | 0.0000983538 | 0.6397747 |
| 11413 | 981.585 | 24.7955 | 0.49 | 2.34 (2.36) | 0.85 | 2.75 (2.79) | 0.0000000000 | 0.7746599 |
| 12264 | 992.416 | 20.3967 | 0.19 | 1.98 (1.95) | 0.41 | 2.03 (2.05) | 0.0010773760 | 0.6100659 |
| 12380 | 994.434 | 25.0743 | 0.29 | 1.95 (1.92) | 0.47 | 2.10 (2.15) | 0.0034709100 | 0.6057611 |
| 12434 | 995.386 | 25.1088 | 0.5 | 2.23 (2.31) | 0.23 | 2.00 (2.03) | 0.0000256872 | 0.6510417 |
| 12993 | 1009.41 | 20.9807 | 0.08 | 1.79 (1.77) | 0.32 | 1.94 (1.89) | 0.0000186629 | 0.6245748 |
| 12998 | 1009.45 | 27.2678 | 0.14 | 1.99 (2.02) | 0.44 | 2.15 (2.17) | 0.0000038202 | 0.6536990 |
| 13145 | 1012.5 | 35.0293 | 0.12 | 1.70 (1.78) | 0.39 | 2.08 (2.18) | 0.0000053017 | 0.6444515 |
| 13342 | 1016.45 | 25.7851 | 0.87 | 2.71 (2.74) | 0.97 | 3.10 (3.12) | 0.0000000115 | 0.7371918 |
| 13429 | 1018.46 | 24.5356 | 0.28 | 2.07 (2.10) | 0.45 | 2.19 (2.27) | 0.0074764750 | 0.5956101 |
| 13746 | 1025.47 | 24.9959 | 0.14 | 2.28 (2.19) | 0.3 | 2.11 (2.12) | 0.0139927000 | 0.5742985 |
| 13747 | 1025.46 | 25.5952 | 0.35 | 2.25 (2.26) | 0.54 | 2.32 (2.39) | 0.0032285030 | 0.6113946 |
| 14047 | 1032.45 | 25.8974 | 0.44 | 2.02 (2.03) | 0.66 | 2.29 (2.33) | 0.0000143904 | 0.6717156 |
| 14071 | 1032.5 | 21.2104 | 0.52 | 2.72 (2.71) | 0.71 | 2.92 (2.96) | 0.0013129670 | 0.6296769 |
| 14478 | 1040.48 | 25.0502 | 0.36 | 2.28 (2.30) | 0.74 | 2.34 (2.39) | 0.0000001908 | 0.7061543 |
| 14751 | 1046.43 | 27.6437 | 0.17 | 1.74 (1.83) | 0.4 | 1.79 (1.88) | 0.0004685956 | 0.6156463 |
| 14763 | 1046.51 | 25.3545 | 0.13 | 2.26 (2.03) | 0.34 | 2.18 (2.06) | 0.0006594612 | 0.6056016 |
| 14906 | 1050.48 | 26.9248 | 0.37 | 2.15 (2.11) | 0.82 | 2.69 (2.71) | 0.0000000000 | 0.8186650 |
| 15216 | 1058.48 | 24.893 | 0.49 | 2.82 (2.90) | 0.13 | 2.13 (1.77) | 0.0000000066 | 0.6974915 |
| 15296 | 1060.38 | 38.31 | 0.31 | 1.76 (1.77) | 0.51 | 1.98 (1.97) | 0.0008959117 | 0.6228741 |
| 15561 | 1065.5 | 25.4545 | 0.04 | 1.76 (1.76) | 0.34 | 2.01 (1.95) | 0.0000000626 | 0.6542304 |
| 15776 | 1068.45 | 24.7645 | 0.96 | 3.25 (3.29) | 0.97 | 3.40 (3.42) | 0.0026429760 | 0.6250000 |
| 15887 | 1069.47 | 26.332 | 0.44 | 1.99 (1.99) | 0.57 | 2.26 (2.24) | 0.0012853300 | 0.6254783 |
| 16059 | 1071.49 | 21.4307 | 0.24 | 2.01 (1.96) | 0.56 | 2.11 (2.10) | 0.0000030793 | 0.6719813 |
| 16168 | 1073.32 | 35.3213 | 0.95 | 3.46 (3.56) | 0.94 | 3.73 (3.81) | 0.0034182630 | 0.6217049 |
| 16773 | 1080.48 | 27.7698 | 0.09 | 1.82 (1.83) | 0.46 | 2.13 (2.12) | 0.0000000031 | 0.6934524 |
| 16779 | 1080.5 | 25.6942 | 0.17 | 2.13 (2.11) | 0.39 | 2.19 (2.22) | 0.0008354786 | 0.6097470 |
| 16976 | 1084.43 | 25.2328 | 0.92 | 3.06 (3.07) | 0.95 | 3.27 (3.36) | 0.0000463093 | 0.6693240 |
| 17045 | 1085.42 | 20.7743 | 0.1 | 2.40 (2.44) | 0.38 | 2.86 (2.83) | 0.0000021539 | 0.6468963 |
| 17694 | 1096.48 | 26.0757 | 0.67 | 3.19 (3.24) | 0.94 | 3.67 (3.69) | 0.0000000002 | 0.7645621 |
| 17830 | 1097.5 | 25.4051 | 0.39 | 2.38 (2.47) | 0.17 | 2.11 (2.19) | 0.0001983307 | 0.6222364 |
| 17899 | 1098.52 | 20.8911 | 0.15 | 2.18 (2.25) | 0.31 | 1.99 (1.97) | 0.0177592900 | 0.5729167 |
| 17968 | 1099.49 | 28.2422 | 0.62 | 2.27 (2.29) | 0.91 | 2.85 (2.86) | 0.0000000000 | 0.8322173 |
| 18029 | 1100.5 | 37.0367 | 0.26 | 1.90 (1.94) | 0.42 | 2.20 (2.17) | 0.0040025990 | 0.6005527 |
| 18077 | 1101.47 | 22.2691 | 0.23 | 2.26 (2.27) | 0.46 | 2.18 (2.19) | 0.0028490630 | 0.6052296 |
| 18358 | 1107.43 | 25.1418 | 0.33 | 2.21 (2.18) | 0.17 | 2.27 (2.31) | 0.0154108000 | 0.5763180 |
| 18604 | 1110.39 | 33.6302 | 0.45 | 2.65 (2.64) | 0.64 | 2.68 (2.77) | 0.0032576420 | 0.6162840 |
| 18833 | 1113.46 | 27.2996 | 0.61 | 2.27 (2.30) | 0.73 | 2.49 (2.53) | 0.0007189701 | 0.6380740 |
| 18939 | 1114.48 | 24.2135 | 0.39 | 2.29 (2.31) | 0.58 | 2.29 (2.36) | 0.0103347600 | 0.5990646 |
| 18943 | 1114.49 | 25.5545 | 0.82 | 3.00 (3.10) | 0.93 | 3.51 (3.57) | 0.0000000137 | 0.7357568 |
| 18988 | 1115.5 | 21.5669 | 0.07 | 2.52 (2.55) | 0.39 | 2.42 (2.46) | 0.0000003678 | 0.6550276 |
| 19046 | 1116.53 | 20.8514 | 0.31 | 2.20 (2.16) | 0.18 | 2.06 (1.96) | 0.0241794300 | 0.5704719 |
| 19137 | 1118.49 | 37.7593 | 0.13 | 2.16 (2.09) | 0.32 | 2.17 (2.11) | 0.0018648940 | 0.5948661 |
| 19284 | 1121.5 | 27.6767 | 0.05 | 1.89 (1.53) | 0.35 | 2.17 (2.17) | 0.0000001361 | 0.6533801 |
| 19655 | 1126.51 | 25.5171 | 0.41 | 2.10 (2.07) | 0.16 | 2.27 (2.35) | 0.0006699383 | 0.6124575 |
| 19773 | 1128.39 | 33.592 | 0.78 | 3.03 (3.03) | 0.83 | 3.31 (3.40) | 0.0000073382 | 0.6856930 |
| 19791 | 1128.49 | 25.6493 | 0.29 | 2.22 (2.23) | 0.46 | 2.37 (2.41) | 0.0045957760 | 0.6021471 |
| 19828 | 1129.46 | 27.9149 | 0.35 | 2.07 (2.04) | 0.69 | 2.39 (2.45) | 0.0000000036 | 0.7308673 |
| 19871 | 1130.34 | 35.393 | 0.53 | 2.54 (2.68) | 0.64 | 2.83 (2.93) | 0.0024305540 | 0.6213861 |
| 20072 | 1134.58 | 23.6558 | 0.54 | 2.31 (2.28) | 0.68 | 2.48 (2.49) | 0.0042056510 | 0.6153805 |
| 20204 | 1137.51 | 26.4263 | 0.11 | 2.01 (1.79) | 0.3 | 1.97 (1.96) | 0.0012198260 | 0.5950787 |
| 20457 | 1138.59 | 19.5068 | 0.19 | 2.05 (1.98) | 0.39 | 2.13 (2.11) | 0.0023397780 | 0.6012436 |
| 20659 | 1139.49 | 20.9651 | 0.33 | 2.58 (2.38) | 0.55 | 2.78 (2.89) | 0.0007335665 | 0.6273384 |
| 20700 | 1140.52 | 25.3846 | 0.22 | 2.05 (2.04) | 0.46 | 2.18 (2.25) | 0.0002634452 | 0.6280825 |
| 20750 | 1141.52 | 24.5065 | 0.4 | 2.17 (2.14) | 0.55 | 2.39 (2.39) | 0.0028613190 | 0.6146365 |
| 20756 | 1141.54 | 37.3328 | 0.35 | 2.12 (2.19) | 0.48 | 2.34 (2.43) | 0.0116587200 | 0.5936437 |
| 20862 | 1143.52 | 36.9674 | 0.31 | 2.01 (1.97) | 0.6 | 2.37 (2.39) | 0.0000007429 | 0.6882440 |
| 20896 | 1144.36 | 35.4858 | 0.11 | 1.85 (1.82) | 0.3 | 2.13 (2.19) | 0.0004876671 | 0.6025191 |
| 21641 | 1155.49 | 20.8473 | 0.28 | 2.30 (2.29) | 0.51 | 2.30 (2.35) | 0.0010064690 | 0.6203763 |
| 21747 | 1157.54 | 37.4441 | 0.62 | 2.52 (2.52) | 0.69 | 2.91 (2.95) | 0.0016402660 | 0.6281888 |
| 21972 | 1160.36 | 35.6006 | 0.89 | 3.07 (3.07) | 0.93 | 3.24 (3.29) | 0.0013345280 | 0.6333440 |
| 22089 | 1162.54 | 20.112 | 0.4 | 2.22 (2.22) | 0.57 | 2.37 (2.40) | 0.0019359780 | 0.6197385 |
| 22725 | 1171.51 | 29.1847 | 0.17 | 1.73 (1.84) | 0.46 | 2.07 (2.06) | 0.0000019286 | 0.6629464 |
| 22835 | 1173.53 | 37.4904 | 0.26 | 2.07 (2.02) | 0.46 | 2.27 (2.21) | 0.0006329833 | 0.6215986 |
| 23224 | 1178.39 | 20.7118 | 0.26 | 2.34 (2.33) | 0.56 | 2.49 (2.48) | 0.0000059388 | 0.6675170 |
| 23356 | 1179.52 | 37.4916 | 0.27 | 2.58 (2.54) | 0.32 | 2.77 (2.76) | 0.0000030780 | 0.6796344 |
| 23409 | 1180.52 | 35.6997 | 0.23 | 2.39 (2.40) | 0.58 | 2.54 (2.61) | 0.0000002173 | 0.6917517 |
| 23423 | 1180.54 | 25.6232 | 0.08 | 1.86 (1.76) | 0.31 | 1.90 (1.83) | 0.0000448896 | 0.6175595 |
| 23518 | 1182.55 | 28.273 | 0.12 | 1.77 (1.83) | 0.55 | 2.00 (2.00) | 0.0000000000 | 0.7296981 |
| 23628 | 1184.56 | 26.4035 | 0.18 | 1.97 (1.91) | 0.45 | 2.01 (2.00) | 0.0000751756 | 0.6355230 |
| 23697 | 1186.53 | 22.3938 | 0.45 | 2.56 (2.57) | 0.89 | 2.83 (2.87) | 0.0000000000 | 0.7924107 |
| 23724 | 1187.36 | 35.6892 | 0.48 | 2.90 (2.88) | 0.82 | 3.01 (3.03) | 0.0000019271 | 0.6936118 |
| 23968 | 1191.52 | 36.1767 | 0.23 | 2.02 (1.97) | 0.46 | 2.35 (2.38) | 0.0002070619 | 0.6308461 |
| 24113 | 1194.55 | 29.2014 | 0.15 | 1.93 (1.98) | 0.33 | 2.02 (2.14) | 0.0024175350 | 0.5948129 |
| 24168 | 1195.48 | 37.5052 | 0.26 | 2.64 (2.73) | 0.24 | 3.00 (3.02) | 0.0117030075 | 0.5953975 |
| 24291 | 1196.33 | 36.1119 | 1 | 4.28 (4.35) | 1 | 4.37 (4.40) | 0.0209884800 | 0.5959821 |
| 24308 | 1196.52 | 21.0014 | 0.4 | 2.78 (2.92) | 0.6 | 2.76 (2.98) | 0.0064923460 | 0.6058673 |
| 24449 | 1199.58 | 21.9516 | 0.63 | 2.55 (2.57) | 0.76 | 2.67 (2.71) | 0.0058900600 | 0.6128827 |
| 24510 | 1200.54 | 24.1426 | 0.55 | 3.47 (3.54) | 0.66 | 3.78 (3.85) | 0.0007539335 | 0.6356293 |
| 24660 | 1203.57 | 26.7602 | 0.13 | 1.84 (1.80) | 0.41 | 2.07 (2.08) | 0.0000041767 | 0.6491815 |
| 24958 | 1209.53 | 26.1548 | 0.43 | 2.54 (2.59) | 0.69 | 2.67 (2.74) | 0.0000464642 | 0.6617772 |
| 25053 | 1211.54 | 25.8213 | 0.26 | 2.20 (2.18) | 0.46 | 1.98 (1.91) | 0.0162144000 | 0.5855655 |
| 25099 | 1212.56 | 21.204 | 0.26 | 2.14 (2.19) | 0.44 | 2.15 (2.21) | 0.0095533380 | 0.5914116 |
| 25294 | 1215.43 | 27.5883 | 0.1 | 2.24 (2.26) | 0.42 | 2.72 (2.76) | 0.0000001331 | 0.6685799 |
| 25295 | 1215.45 | 26.8785 | 0.21 | 2.73 (2.73) | 0.43 | 2.85 (2.87) | 0.0007649960 | 0.6158057 |
| 25363 | 1216.54 | 24.2435 | 0.82 | 2.84 (2.84) | 0.77 | 3.15 (3.14) | 0.0124830400 | 0.6034226 |
| 25429 | 1217.53 | 35.7806 | 0.26 | 2.95 (3.00) | 0.43 | 3.25 (3.34) | 0.0050767050 | 0.5983737 |
| 25866 | 1223.55 | 27.457 | 0.27 | 2.22 (2.24) | 0.51 | 2.09 (2.14) | 0.0021463770 | 0.6119260 |
| 26098 | 1225.32 | 35.6881 | 0.46 | 2.46 (2.52) | 0.3 | 2.35 (2.53) | 0.0219019800 | 0.5832802 |
| 26163 | 1226.53 | 21.0162 | 0.58 | 2.67 (2.59) | 0.83 | 2.77 (2.86) | 0.0000251304 | 0.6729379 |
| 26412 | 1231.42 | 21.5127 | 0.14 | 2.52 (2.45) | 0.4 | 2.59 (2.66) | 0.0000686967 | 0.6290391 |
| 26670 | 1235.56 | 26.6462 | 0.5 | 2.76 (2.70) | 0.57 | 3.34 (3.32) | 0.0003005027 | 0.6425914 |
| 26879 | 1238.56 | 21.1369 | 0.2 | 2.35 (2.35) | 0.43 | 2.23 (2.24) | 0.0024420830 | 0.6037415 |
| 27350 | 1247.52 | 22.0008 | 0.79 | 3.00 (3.03) | 0.98 | 3.06 (3.09) | 0.0008800471 | 0.6381803 |
| 27742 | 1251.62 | 22.5253 | 0.21 | 2.47 (2.47) | 0.42 | 2.41 (2.43) | 0.0038828430 | 0.5988520 |
| 28005 | 1255.48 | 35.7697 | 0.41 | 2.94 (3.04) | 0.45 | 2.80 (2.90) | 0.0032727805 | 0.6118463 |
| 28103 | 1257.44 | 33.9189 | 0.89 | 3.49 (3.52) | 0.91 | 3.63 (3.73) | 0.0026627180 | 0.6248406 |
| 28132 | 1257.64 | 19.9186 | 0.42 | 2.82 (2.83) | 0.06 | 2.41 (2.56) | 0.0000000055 | 0.6821854 |
| 28281 | 1260.56 | 21.8281 | 0.67 | 2.71 (2.72) | 0.58 | 2.54 (2.52) | 0.0230625500 | 0.5920493 |
| 28385 | 1262.47 | 38.233 | 0.19 | 2.41 (2.50) | 0.43 | 2.27 (2.29) | 0.0014743870 | 0.6082589 |
| 28466 | 1263.54 | 22.7286 | 0.37 | 2.61 (2.66) | 0.83 | 2.68 (2.79) | 0.0000000002 | 0.7566964 |
| 28561 | 1265.59 | 27.0867 | 0.49 | 3.20 (3.31) | 0.81 | 3.74 (3.76) | 0.0000000000 | 0.7761480 |
| 28669 | 1267.54 | 21.1725 | 0.15 | 2.43 (2.42) | 0.38 | 2.35 (2.36) | 0.0008854221 | 0.6070366 |
| 28747 | 1268.57 | 27.2482 | 0.95 | 3.83 (3.98) | 0.93 | 3.61 (3.68) | 0.0035170740 | 0.6213329 |
| 28850 | 1270.55 | 29.3808 | 0.15 | 2.13 (2.10) | 0.39 | 2.21 (2.21) | 0.0001818892 | 0.6213329 |
| 29279 | 1276.4 | 35.9233 | 0.97 | 3.51 (3.55) | 0.98 | 3.62 (3.70) | 0.0215629100 | 0.5955570 |
| 29538 | 1281.59 | 27.0902 | 0.26 | 2.33 (2.35) | 0.49 | 2.27 (2.34) | 0.0022367390 | 0.6101722 |
| 29685 | 1283.55 | 27.2665 | 0.17 | 2.14 (2.11) | 0.5 | 2.29 (2.28) | 0.0000004753 | 0.6759141 |
| 29823 | 1286.42 | 36.407 | 0.15 | 1.80 (1.79) | 0.35 | 1.85 (1.90) | 0.0010921080 | 0.6036352 |
| 29840 | 1286.54 | 29.4051 | 0.15 | 2.05 (2.06) | 0.36 | 1.97 (1.99) | 0.0018470090 | 0.5995429 |
| 29906 | 1287.59 | 21.8737 | 0.49 | 2.79 (2.81) | 0.75 | 2.72 (2.76) | 0.0054309300 | 0.6123512 |
| 30136 | 1292.39 | 36.1593 | 0.26 | 2.18 (2.17) | 0.4 | 2.28 (2.31) | 0.0174105900 | 0.5822704 |
| 30524 | 1296.61 | 21.6926 | 0.34 | 2.41 (2.35) | 0.19 | 2.12 (2.04) | 0.0092001500 | 0.5838648 |
| 30575 | 1297.58 | 27.365 | 0.33 | 3.02 (3.08) | 0.85 | 3.07 (3.14) | 0.0000000000 | 0.7771046 |
| 30699 | 1299.58 | 22.3818 | 0.39 | 2.50 (2.55) | 0.84 | 2.52 (2.55) | 0.0000000030 | 0.7392645 |
| 31275 | 1310.59 | 21.3883 | 0.11 | 2.07 (2.05) | 0.33 | 1.97 (1.92) | 0.0003532994 | 0.6079932 |
| 31480 | 1312.55 | 29.7677 | 0.89 | 3.08 (3.12) | 0.95 | 3.31 (3.32) | 0.0000001045 | 0.7210353 |
| 31524 | 1312.62 | 20.0087 | 0.21 | 2.54 (2.54) | 0.45 | 2.38 (2.33) | 0.0028320880 | 0.6037946 |
| 32022 | 1319.58 | 20.8863 | 0.16 | 2.23 (2.26) | 0.41 | 2.25 (2.28) | 0.0001966677 | 0.6230867 |
| 32171 | 1321.59 | 28.371 | 0.97 | 3.77 (3.87) | 0.96 | 4.16 (4.20) | 0.0000859358 | 0.6632653 |
| 32336 | 1324.57 | 21.266 | 0.13 | 2.41 (2.34) | 0.39 | 2.47 (2.53) | 0.0000459736 | 0.6302615 |
| 32343 | 1324.59 | 28.7015 | 0.23 | 2.36 (2.20) | 0.39 | 2.47 (2.52) | 0.0201334700 | 0.5791348 |
| 32470 | 1326.55 | 29.2043 | 0.35 | 2.17 (2.20) | 0.72 | 2.44 (2.45) | 0.0000000017 | 0.7372449 |
| 32471 | 1326.56 | 27.1105 | 0.19 | 2.95 (2.96) | 0.39 | 2.82 (2.84) | 0.0120913800 | 0.5834928 |
| 32481 | 1326.57 | 21.6723 | 0.17 | 2.27 (2.30) | 0.4 | 2.10 (2.11) | 0.0019720600 | 0.6023065 |
| 32823 | 1332.54 | 21.7375 | 0.43 | 2.86 (2.86) | 0.15 | 2.44 (2.30) | 0.0000020462 | 0.6579507 |
| 32874 | 1333.42 | 36.108 | 0.46 | 2.17 (2.26) | 0.7 | 2.19 (2.25) | 0.0016362000 | 0.6259035 |
| 33135 | 1338.6 | 23.9878 | 0.83 | 2.68 (2.73) | 0.7 | 2.57 (2.63) | 0.0101057300 | 0.6062394 |
| 33209 | 1339.6 | 27.4859 | 0.45 | 2.17 (2.21) | 0.65 | 2.26 (2.29) | 0.0012040470 | 0.6281888 |
| 33238 | 1340.6 | 28.3984 | 0.3 | 2.07 (2.09) | 0.49 | 1.96 (1.95) | 0.0210689400 | 0.5844494 |
| 33812 | 1352.56 | 29.7655 | 0.23 | 2.04 (2.00) | 0.63 | 2.21 (2.22) | 0.0000000171 | 0.7113095 |
| 33938 | 1353.59 | 21.483 | 0.68 | 2.70 (2.73) | 0.56 | 2.45 (2.43) | 0.0009135419 | 0.6341412 |
| 33973 | 1353.66 | 25.6316 | 0.68 | 2.38 (2.41) | | 2.56 (2.61) | 0.0000015715 | 0.6986076 |
| 34009 | 1354.62 | 35.7097 | 0.34 | 2.44 (2.49) | 0.03 | 2.41 (2.32) | 0.0000000548 | 0.6532207 |
| 34017 | 1354.64 | 22.1393 | 0.37 | 2.29 (2.29) | 0.19 | 2.08 (2.08) | 0.0020464320 | 0.6012968 |
| 34267 | 1359.61 | 23.1947 | 0.19 | 2.26 (2.31) | 0.44 | 2.13 (2.14) | 0.0011500350 | 0.6112883 |
| 34339 | 1361.63 | 21.9874 | 0.42 | 2.80 (2.84) | 0.73 | 2.66 (2.68) | 0.0015877640 | 0.6260629 |
| 34432 | 1363.43 | 36.3375 | 0.98 | 3.26 (3.30) | 1 | 3.38 (3.38) | 0.0076121000 | 0.6109694 |
| 34766 | 1367.64 | 38.8826 | 0.72 | 2.54 (2.60) | 0.81 | 2.71 (2.74) | 0.0113675900 | 0.6045918 |
| 35204 | 1375.61 | 23.245 | 0.23 | 2.21 (2.29) | 0.57 | 2.17 (2.19) | 0.0000129376 | 0.6607674 |
| 35339 | 1378.61 | 28.822 | 0.95 | 3.26 (3.29) | 1 | 3.50 (3.50) | 0.0000000000 | 0.7731718 |
| 35424 | 1380.64 | 23.8305 | 0.51 | 2.47 (2.49) | 0.49 | 2.31 (2.37) | 0.0040088845 | 0.6776946 |
| 35550 | 1383.64 | 38.9402 | 0.15 | 2.17 (2.18) | 0.32 | 2.12 (2.22) | 0.0080177690 | 0.5822173 |
| 35979 | 1390.44 | 36.9459 | 0.97 | 4.29 (4.31) | 0.96 | 4.47 (4.48) | 0.0050406960 | 0.6166029 |
| 36500 | 1399.62 | 28.7444 | 0.18 | 2.10 (2.15) | 0.33 | 2.24 (2.24) | 0.0096953840 | 0.5826956 |
| 36566 | 1401.38 | 36.5567 | 0.2 | 2.74 (2.85) | 0.23 | 2.80 (3.14) | 0.0123441870 | 0.5840508 |
| 36586 | 1401.66 | 40.0387 | 0.23 | 2.28 (2.29) | 0.45 | 1.95 (1.97) | 0.0149929900 | 0.5854060 |
| 36759 | 1405.61 | 39.0354 | 0.42 | 2.71 (2.76) | 0.44 | 2.63 (2.67) | 0.0074969616 | 0.6299692 |
| 36769 | 1405.64 | 20.1391 | 0.19 | 2.35 (2.39) | 0.52 | 2.45 (2.45) | 0.0000009332 | 0.6745323 |
| 36868 | 1407.66 | 37.2292 | 0.34 | 2.16 (2.23) | 0.6 | 2.38 (2.45) | 0.0000138609 | 0.6666135 |
| 36988 | 1408.66 | 39.1338 | 0.47 | 2.46 (2.57) | 0.67 | 2.62 (2.74) | 0.0016949570 | 0.6251063 |
| 37056 | 1409.58 | 22.0427 | 0.96 | 3.96 (4.00) | 1 | 4.03 (4.04) | 0.0080274110 | 0.6102253 |
| 37124 | 1410.62 | 19.897 | 0.2 | 2.13 (2.14) | 0.36 | 2.21 (2.16) | 0.0109983100 | 0.5840774 |
| 37340 | 1415.64 | 23.5532 | 0.34 | 2.31 (2.38) | 0.05 | 2.07 (2.03) | 0.0000004340 | 0.6455676 |
| 37603 | 1421.49 | 37.0163 | 0.16 | 2.45 (2.51) | 0.35 | 2.43 (2.42) | 0.0031188770 | 0.5944940 |
| 37610 | 1421.59 | 38.7131 | 0.09 | 1.93 (1.94) | 0.18 | 1.80 (1.76) | 0.0138518835 | 0.5939626 |
| 37662 | 1422.6 | 21.7229 | 0.82 | 3.49 (3.56) | 0.99 | 3.47 (3.46) | 0.0245848900 | 0.5934311 |
| 37903 | 1424.66 | 39.2996 | 0.86 | 2.93 (2.94) | 0.98 | 3.18 (3.22) | 0.0000149198 | 0.6799532 |
| 37949 | 1425.59 | 22.3178 | 0.84 | 3.46 (3.40) | 0.98 | 3.56 (3.62) | 0.0001700346 | 0.6562500 |
| 38011 | 1426.64 | 22.4206 | 0.09 | 1.97 (1.82) | 0.36 | 2.09 (2.20) | 0.0000039479 | 0.6406781 |
| 38586 | 1435.51 | 19.7504 | 0.08 | 1.97 (2.06) | 0.34 | 2.29 (2.30) | 0.0000039634 | 0.6369048 |
| 38752 | 1438.45 | 36.7639 | 0.95 | 3.67 (3.70) | 1 | 3.82 (3.81) | 0.0017528780 | 0.6301020 |
| 38780 | 1438.66 | 30.2014 | 0.21 | 2.45 (2.55) | 0.54 | 2.43 (2.52) | 0.0000075611 | 0.6619898 |
| 38790 | 1438.66 | 22.0085 | 0.45 | 2.54 (2.59) | 0.33 | 2.25 (2.20) | 0.0182506400 | 0.5864158 |
| 38798 | 1438.67 | 27.8755 | 0.88 | 3.38 (3.47) | 1 | 3.58 (3.60) | 0.0000188571 | 0.6778274 |
| 38879 | 1439.66 | 29.8171 | 0.6 | 2.59 (2.40) | 0.44 | 2.25 (2.21) | 0.0020986910 | 0.6206420 |
| 38910 | 1440.56 | 24.3004 | 0.3 | 2.56 (2.63) | 0.63 | 2.48 (2.52) | 0.0000598345 | 0.6530612 |
| 38951 | 1440.66 | 39.2845 | 0.4 | 2.38 (2.32) | 0.56 | 2.51 (2.54) | 0.0084365000 | 0.6015094 |
| 39064 | 1442.63 | 27.6328 | 0.55 | 2.55 (2.60) | 0.32 | 2.04 (1.99) | 0.0000119912 | 0.6650723 |
| 39275 | 1445.62 | 37.359 | 0.28 | 2.34 (2.42) | 0.28 | 2.58 (2.72) | 0.0255921700 | 0.5786565 |
| 39322 | 1446.64 | 39.4279 | 0.37 | 2.90 (2.89) | 0.28 | 3.06 (3.26) | 0.0255586050 | 0.5734216 |
| 39607 | 1447.7 | 19.4724 | 0.57 | 2.55 (2.60) | 0.71 | 2.66 (2.75) | 0.0225749800 | 0.5925808 |
| 40091 | 1449.64 | 21.8561 | 0.95 | 3.57 (3.58) | 0.99 | 3.79 (3.80) | 0.0000873287 | 0.6631059 |
| 40294 | 1452.66 | 23.6065 | 0.68 | 2.66 (2.68) | 0.65 | 2.46 (2.50) | 0.0240458800 | 0.5920493 |
| 40487 | 1457.6 | 21.9348 | 0.16 | 2.37 (2.53) | 0.44 | 2.23 (2.25) | 0.0001140187 | 0.6298895 |
| 40541 | 1458.63 | 27.9416 | 0.22 | 2.46 (2.52) | 0.41 | 2.23 (2.31) | 0.0150628200 | 0.5832270 |
| 40737 | 1462.62 | 39.4214 | 0.47 | 2.97 (3.08) | 0.34 | 3.29 (3.41) | 0.0075314231 | 0.6511480 |
| 41434 | 1466.65 | 28.5188 | 0.35 | 2.33 (2.26) | 0.72 | 2.55 (2.65) | 0.0000000262 | 0.7190689 |
| 41471 | 1467.67 | 21.2878 | 0.37 | 2.48 (2.40) | 0.57 | 2.54 (2.55) | 0.0030419290 | 0.6136267 |
| 41601 | 1469.67 | 23.6936 | 0.94 | 3.41 (3.46) | 0.99 | 3.41 (3.41) | 0.0022747760 | 0.6135204 |
| 41665 | 1470.68 | 21.0804 | 0.24 | 2.04 (2.11) | 0.48 | 2.02 (1.99) | 0.0015076230 | 0.6134141 |
| 41972 | 1478.61 | 39.3027 | 0.47 | 2.42 (2.49) | 0.42 | 2.53 (2.61) | 0.0007689901 | 0.6213595 |
| 42188 | 1483.66 | 22.586 | 0.11 | 2.54 (2.47) | 0.36 | 2.70 (2.75) | 0.0000303571 | 0.6293048 |
| 42216 | 1483.7 | 20.6531 | 0.43 | 2.64 (2.60) | 0.65 | 2.59 (2.58) | 0.0100821900 | 0.6015094 |
| 42378 | 1486.68 | 21.1523 | 0.37 | 2.39 (2.46) | 0.7 | 2.51 (2.49) | 0.0000022721 | 0.6861182 |
| 42404 | 1487.65 | 29.6217 | 0.38 | 2.43 (2.47) | 0.75 | 2.69 (2.78) | 0.0000000020 | 0.7386267 |
| 42594 | 1491.74 | 39.8339 | 0.55 | 2.37 (2.40) | 0.71 | 2.62 (2.65) | 0.0004114142 | 0.6430697 |
| 42722 | 1493.63 | 21.8415 | 0.27 | 2.41 (2.38) | 0.5 | 2.35 (2.34) | 0.0021249810 | 0.6116071 |
| 42776 | 1494.66 | 30.3993 | 0.49 | 2.31 (2.33) | 0.72 | 2.35 (2.36) | 0.0013611540 | 0.6289328 |
| 42866 | 1496.68 | 30.3745 | 0.23 | 2.26 (2.24) | 0.57 | 2.16 (2.14) | 0.0000141857 | 0.6600234 |
| 42867 | 1496.63 | 22.3363 | 0.2 | 2.54 (2.49) | 0.39 | 2.26 (2.23) | 0.0251830900 | 0.5749362 |
| 43226 | 1503.66 | 29.682 | 0.11 | 1.83 (1.94) | 0.31 | 2.19 (2.13) | 0.0002778785 | 0.6078869 |
| 43442 | 1507.74 | 40.0239 | 0.93 | 3.08 (3.11) | 0.94 | 3.24 (3.27) | 0.0121397400 | 0.6042730 |
| 43543 | 1508.68 | 29.3327 | 0.59 | 3.09 (3.26) | 0.93 | 3.35 (3.43) | 0.0000000016 | 0.7491497 |
| 43605 | 1509.79 | 29.5021 | 0.5 | 3.66 (3.69) | 0.2 | 3.57 (3.48) | 0.0000210746 | 0.6506696 |
| 43828 | 1512.69 | 26.6212 | 0.45 | 2.21 (2.22) | 0.26 | 2.00 (2.01) | 0.0009446625 | 0.6176658 |
| 43851 | 1513.44 | 36.7892 | 0.17 | 2.57 (2.71) | 0.47 | 2.44 (2.44) | 0.0000464513 | 0.6401467 |
| 43863 | 1513.63 | 29.5129 | 0.03 | 2.18 (2.06) | 0.3 | 2.09 (2.09) | 0.0000004916 | 0.6351509 |
| 44146 | 1518.6 | 19.3715 | 0.16 | 2.35 (2.31) | 0.47 | 2.46 (2.50) | 0.0000030478 | 0.6597577 |
| 44464 | 1521.69 | 30.5282 | 0.27 | 2.11 (2.16) | 0.54 | 2.14 (2.18) | 0.0001171246 | 0.6420068 |
| 44592 | 1523.67 | 21.9661 | 0.81 | 3.58 (3.61) | 0.85 | 3.72 (3.79) | 0.0006406686 | 0.6415816 |
| 44633 | 1523.84 | 29.7538 | 0.36 | 3.01 (3.00) | 0.63 | 3.05 (3.06) | 0.0004456704 | 0.6359481 |
| 44679 | 1524.65 | 20.0263 | 0.58 | 2.69 (2.69) | 0.9 | 2.76 (2.80) | 0.0000037168 | 0.6905825 |
| 44802 | 1526.69 | 23.9186 | 0.44 | 2.26 (2.27) | 0.57 | 2.03 (1.99) | 0.0250716150 | 0.5838117 |
| 45021 | 1532.62 | 26.3459 | 0.27 | 2.71 (2.77) | 0.33 | 2.37 (2.34) | 0.0249947450 | 0.5750691 |
| 45243 | 1535.68 | 22.0021 | 0.13 | 2.72 (2.72) | 0.4 | 2.51 (2.45) | 0.0001166257 | 0.6240434 |
| 45980 | 1552.5 | 37.2147 | 0.9 | 3.34 (3.36) | 0.97 | 3.45 (3.48) | 0.0023851090 | 0.6262755 |
| 46091 | 1554.66 | 28.5929 | 0.18 | 2.06 (2.05) | 0.51 | 2.02 (2.02) | 0.0000078191 | 0.6576318 |
| 46184 | 1556.74 | 40.0323 | 0.1 | 2.13 (2.15) | 0.48 | 2.36 (2.33) | 0.0000000029 | 0.6974915 |
| 46338 | 1560.58 | 21.7691 | 0.31 | 2.56 (2.55) | 0.68 | 2.67 (2.64) | 0.0000002077 | 0.7009991 |
| 46369 | 1560.7 | 29.7854 | 0.35 | 2.18 (2.06) | 0.44 | 2.34 (2.32) | 0.0020891023 | 0.6579241 |
| 46542 | 1561.45 | 36.9566 | 0.5 | 2.42 (2.49) | 0.68 | 2.52 (2.53) | 0.0041779970 | 0.6148491 |
| 46606 | 1562.69 | 22.4615 | 0.56 | 3.15 (3.19) | 0.72 | 3.15 (3.23) | 0.0186428400 | 0.5955038 |
| 46725 | 1564.72 | 28.37 | 0.33 | 2.10 (2.08) | 0.57 | 1.92 (1.92) | 0.0150134700 | 0.5922619 |
| 46756 | 1565.69 | 26.3032 | 0.66 | 2.46 (2.46) | 0.72 | 2.10 (2.14) | 0.0016225150 | 0.6290923 |
| 47285 | 1575.75 | 30.1998 | 0.4 | 2.28 (2.30) | 0.22 | 2.14 (1.99) | 0.0042637120 | 0.5973108 |
| 47402 | 1576.67 | 43.3003 | 0.52 | 3.33 (3.34) | 0.32 | 3.35 (3.49) | 0.0194634200 | 0.5873193 |
| 47855 | 1576.74 | 19.5079 | 0.43 | 2.58 (2.56) | 0.66 | 2.69 (2.77) | 0.0004905807 | 0.6377551 |
| 48394 | 1583.7 | 23.2678 | 0.51 | 2.57 (2.60) | 0.79 | 2.62 (2.66) | 0.0001287317 | 0.6557185 |
| 48417 | 1584.51 | 37.4959 | 0.65 | 2.50 (2.50) | 0.82 | 2.53 (2.55) | 0.0202491900 | 0.5956633 |
| 48520 | 1586.74 | 28.8819 | 0.44 | 2.30 (2.31) | 0.69 | 2.19 (2.22) | 0.0235215000 | 0.5901361 |
| 48580 | 1588.71 | 30.1503 | 0.57 | 2.65 (2.67) | 0.79 | 2.79 (2.87) | 0.0001973546 | 0.6522109 |
| 48751 | 1592.7 | 22.1803 | 0.37 | 2.69 (2.75) | 0.83 | 2.65 (2.63) | 0.0000000180 | 0.7262968 |
| 49122 | 1592.73 | 19.5192 | 0.27 | 2.37 (2.37) | 0.57 | 2.44 (2.46) | 0.0000159179 | 0.6607143 |
| 49243 | 1593.69 | 22.3763 | 0.16 | 2.43 (2.33) | 0.48 | 2.29 (2.31) | 0.0000101356 | 0.6518920 |
| 49295 | 1594.73 | 23.0529 | 0.81 | 2.71 (2.74) | 0.81 | 2.41 (2.43) | 0.0003126345 | 0.6493410 |
| 49334 | 1595.69 | 20.1503 | 0.31 | 2.63 (2.65) | 0.53 | 2.53 (2.52) | 0.0094888380 | 0.5966199 |
| 49608 | 1602.74 | 28.9718 | 0.54 | 2.29 (2.29) | 0.74 | 2.37 (2.37) | 0.0018234020 | 0.6265412 |
| 49713 | 1604.73 | 30.3377 | 0.11 | 2.33 (2.27) | 0.36 | 2.16 (2.18) | 0.0000922588 | 0.6212266 |
| 49948 | 1608.68 | 22.349 | 0.3 | 2.47 (2.46) | 0.73 | 2.30 (2.34) | 0.0000012871 | 0.6890944 |
| 49958 | 1608.73 | 30.9321 | 0.34 | 2.61 (2.62) | 0.85 | 2.66 (2.68) | 0.0000000000 | 0.7680697 |
| 50008 | 1609.75 | 30.2043 | 0.35 | 2.63 (2.73) | 0.56 | 2.70 (2.82) | 0.0016678800 | 0.6195791 |
| 50056 | 1610.87 | 30.2917 | 0.58 | 3.31 (3.33) | 0.4 | 3.21 (3.22) | 0.0043033220 | 0.6105442 |
| 50172 | 1612.76 | 23.3804 | 0.2 | 2.29 (2.25) | 0.4 | 2.10 (2.10) | 0.0136802800 | 0.5830145 |
| 50212 | 1613.82 | 23.986 | 0.57 | 2.44 (2.47) | 0.79 | 2.41 (2.45) | 0.0134503200 | 0.6010842 |
| 50384 | 1616.73 | 40.25 | 0.31 | 2.66 (2.83) | 0.15 | 2.63 (2.61) | 0.0080769160 | 0.5807823 |
| 50638 | 1620.7 | 22.6628 | 0.31 | 2.49 (2.51) | 0.6 | 2.52 (2.62) | 0.0000393812 | 0.6563563 |
| 50766 | 1622.71 | 30.44 | 0.22 | 2.26 (2.21) | 0.39 | 2.12 (2.09) | 0.0234409600 | 0.5767432 |
| 50838 | 1623.73 | 29.8577 | 0.36 | 2.43 (2.55) | 0.25 | 1.93 (1.84) | 0.0219462100 | 0.5775935 |
| 50840 | 1623.73 | 24.1239 | 0.89 | 3.89 (3.92) | 0.99 | 3.69 (3.76) | 0.0004943547 | 0.6448236 |
| 50904 | 1624.55 | 37.7263 | 0.86 | 2.89 (2.89) | 0.99 | 3.14 (3.17) | 0.0000007470 | 0.7056760 |
| 51175 | 1630.74 | 20.6473 | 0.18 | 2.08 (2.16) | 0.46 | 2.30 (2.27) | 0.0000234887 | 0.6455145 |
| 51328 | 1632.71 | 40.147 | 0.42 | 2.62 (2.76) | 0.27 | 2.50 (2.63) | 0.0259641400 | 0.5785502 |
| 51838 | 1635.65 | 37.3773 | 0.1 | 2.14 (2.15) | 0.35 | 2.37 (2 43) | 0.0000198042 | 0.6301020 |
| 51865 | 1635.76 | 30.3357 | 0.7 | 2.96 (3.08) | 0.75 | 3.13 (3.28) | 0.0151952800 | 0.5999150 |
| 51916 | 1636.7 | 20.0325 | 0.43 | 2.69 (2.78) | 0.84 | 2.95 (3.04) | 0.0000000002 | 0.7581845 |
| 52189 | 1640.58 | 23.2418 | 0.87 | 3.54 (3.59) | 0.98 | 3.74 (3.72) | 0.0005884920 | 0.6428571 |
| 52320 | 1643.74 | 29.5324 | 0.67 | 2.42 (2.48) | 0.88 | 2.43 (2.47) | 0.0249601400 | 0.5926871 |
| 52446 | 1645.76 | 20.6137 | 0.21 | 2.31 (2.24) | 0.4 | 2.29 (2.34) | 0.0072085550 | 0.5909864 |
| 52730 | 1649.71 | 19.5786 | 0.15 | 2.03 (1.96) | 0.38 | 2.37 (2.41) | 0.0001090014 | 0.6245748 |
| 52769 | 1649.73 | 22.643 | 0.7 | 2.72 (2.72) | 0.88 | 2.95 (2.99) | 0.0000056420 | 0.6878189 |
| 53328 | 1657.72 | 22.8973 | 0.19 | 2.34 (2.28) | 0.38 | 2.29 (2.09) | 0.0085484930 | 0.5869473 |
| 53375 | 1658.59 | 21.4575 | 0.18 | 2.51 (2.46) | 0.32 | 2.55 (2.63) | 0.0212289500 | 0.5731293 |
| 53393 | 1658.73 | 29.9416 | 0.38 | 2.50 (2.44) | 0.51 | 2.73 (2.82) | 0.0093803080 | 0.5982674 |
| 53744 | 1666.78 | 30.6643 | 0.33 | 2.29 (2.28) | 0.63 | 2.33 (2.36) | 0.0000166223 | 0.6654974 |
| 53800 | 1667.79 | 40.5608 | 0.57 | 2.47 (2.51) | 0.39 | 2.52 (2.51) | 0.0260373100 | 0.5857781 |
| 53866 | 1668.81 | 40.4656 | 0.35 | 2.50 (2.51) | 0.53 | 2.47 (2.49) | 0.0162219300 | 0.5906675 |
| 53957 | 1669.69 | 21.4647 | 0.55 | 2.60 (2.58) | 0.82 | 2.54 (2.51) | 0.0024596760 | 0.6239371 |
| 54184 | 1673.76 | 40.717 | 0.39 | 3.36 (3.46) | 0.24 | 3.04 (3.25) | 0.0145539800 | 0.5836522 |
| 54269 | 1675.71 | 29.2032 | 0.23 | 1.97 (1.96) | 0.41 | 1.89 (1.91) | 0.0215014400 | 0.5791348 |
| 54421 | 1679.7 | 22.6046 | 0.28 | 2.36 (2.34) | 0.52 | 2.37 (2.25) | 0.0016443140 | 0.6156463 |
| 54438 | 1679.95 | 23.8176 | 0.83 | 3.79 (3.89) | 0.92 | 4.00 (4.00) | 0.0151641500 | 0.6008716 |
| 54703 | 1684.71 | 29.6453 | 0.19 | 2.38 (2.45) | 0.35 | 2.46 (2.43) | 0.0120523100 | 0.5819515 |
| 55001 | 1689.8 | 27.5541 | 0.18 | 2.22 (2.16) | 0.41 | 2.08 (2.12) | 0.0016836850 | 0.6051233 |
| 55143 | 1692.8 | 30.8875 | 0.43 | 2.52 (2.54) | 0.7 | 2.80 (2.79) | 0.0000029344 | 0.6860119 |
| 55314 | 1693.76 | 20.5058 | 0.27 | 2.35 (2.30) | 0.58 | 2.31 (2.35) | 0.0000213348 | 0.6588010 |
| 55315 | 1693.76 | 23.4788 | 0.13 | 2.16 (2.11) | 0.34 | 1.98 (1.99) | 0.0014751570 | 0.5985863 |
| 55450 | 1694.78 | 23.5864 | 0.15 | 2.22 (2.21) | 0.3 | 2.07 (2.10) | 0.0251530700 | 0.5682929 |
| 55484 | 1695.73 | 23.1763 | 0.21 | 2.27 (2.40) | 0.39 | 2.12 (2.10) | 0.0181332800 | 0.5795599 |
| 55516 | 1696.72 | 23.9499 | 0.41 | 2.44 (2.42) | 0.46 | 2.22 (2.22) | 0.0093334656 | 0.6117400 |
| 55582 | 1697.74 | 30.8785 | 0.87 | 2.86 (2.88) | 0.94 | 3.04 (3.04) | 0.0005336511 | 0.6439201 |
| 55917 | 1703.84 | 33.5849 | 0.23 | 2.21 (2.24) | 0.51 | 2.47 (2.41) | 0.0000301684 | 0.6503508 |
| 56011 | 1705.73 | 40.4367 | 0.46 | 2.39 (2.54) | 0.31 | 2.30 (2.30) | 0.0219868400 | 0.5835459 |
| 56099 | 1707.81 | 23.5945 | 0.23 | 2.36 (2.33) | 0.45 | 2.18 (2.24) | 0.0052215560 | 0.5980548 |
| 56139 | 1708.79 | 30.5905 | 0.71 | 2.71 (2.73) | 0.89 | 2.95 (3.00) | 0.0000427471 | 0.6694303 |
| 56493 | 1716.66 | 20.1773 | 0.24 | 2.51 (2.61) | 0.7 | 2.69 (2.71) | 0.0000000001 | 0.7493622 |
| 56514 | 1716.77 | 27.9978 | 0.62 | 2.50 (2.56) | 0.88 | 2.64 (2.71) | 0.0000116213 | 0.6808036 |
| 56662 | 1720.69 | 19.6657 | 0.11 | 2.16 (2.17) | 0.44 | 2.40 (2.44) | 0.0000001596 | 0.6710247 |
| 56806 | 1723.52 | 37.744 | 0.34 | 2.15 (2.12) | 0.3 | 1.90 (1.88) | 0.0000003493 | 0.6896790 |
| 56884 | 1725.59 | 38.322 | 0.9 | 2.98 (2.99) | 1 | 3.35 (3.36) | 0.0000005390 | 0.7083333 |
| 56992 | 1728.77 | 36.767 | 0.41 | 2.33 (2.28) | 0.66 | 2.35 (2.36) | 0.0007689808 | 0.6324405 |
| 57265 | 1732.77 | 28.1753 | 0.8 | 3.27 (3.28) | 0.93 | 3.36 (3.42) | 0.0001819046 | 0.6553997 |
| 57360 | 1734.66 | 19.8976 | 0.1 | 2.19 (2.22) | 0.44 | 2.32 (2.32) | 0.0000001293 | 0.6711310 |
| 57531 | 1737.78 | 31.0012 | 0.84 | 2.99 (3.01) | 0.94 | 3.37 (3.46) | 0.0000000119 | 0.7368197 |
| 57537 | 1737.78 | 23.7334 | 0.91 | 3.63 (3.64) | 0.99 | 3.73 (3.74) | 0.0009004033 | 0.6380208 |
| 57888 | 1746.59 | 38.2127 | 0.33 | 2.35 (2.41) | 0.54 | 2.32 (2.32) | 0.0050878590 | 0.6053359 |
| 58050 | 1749.81 | 30.6146 | 0.45 | 2.46 (2.50) | 0.74 | 2.57 (2.64) | 0.0000070152 | 0.6803784 |
| 58084 | 1750.78 | 23.8278 | 0.58 | 2.77 (2.69) | 0.9 | 2.91 (2.99) | 0.0000003278 | 0.7103529 |
| 58355 | 1754.9 | 31.2589 | 0.76 | 3.61 (3.61) | 0.88 | 3.75 (3.78) | 0.0010274980 | 0.6360544 |
| 58633 | 1761.69 | 19.3667 | 0.12 | 2.26 (2.34) | 0.32 | 2.32 (2.29) | 0.0008152895 | 0.6011905 |
| 58759 | 1761.83 | 21.6467 | 0.44 | 2.43 (2.43) | 0.33 | 2.15 (2.10) | 0.0248364200 | 0.5818452 |
| 58880 | 1764.68 | 19.9065 | 0.23 | 2.41 (2.49) | 0.54 | 2.60 (2.62) | 0.0000044648 | 0.6673044 |
| 58941 | 1765.81 | 31.0002 | 0.7 | 3.05 (3.07) | 0.84 | 3.23 (3.25) | 0.0001389579 | 0.6574724 |
| 59142 | 1769.71 | 28.1352 | 0.89 | 2.71 (2.74) | 0.89 | 2.51 (2.51) | 0.0057067880 | 0.6148491 |
| 59149 | 1769.78 | 30.9769 | 0.32 | 2.34 (2.36) | 0.19 | 2.05 (1.93) | 0.0187329400 | 0.5746173 |
| 59295 | 1773.82 | 34.6048 | 0.31 | 2.00 (2.00) | 0.6 | 2.14 (2.16) | 0.0000091909 | 0.6686862 |
| 59368 | 1775.79 | 31.4772 | 0.44 | 2.69 (2.80) | 0.26 | 2.56 (2.45) | 0.0053703670 | 0.5986395 |
| 59773 | 1783.79 | 39.8152 | 0.33 | 2.33 (2.31) | 0.63 | 2.42 (2.49) | 0.0000091630 | 0.6704932 |
| 59928 | 1788.84 | 29.8728 | 0.28 | 2.44 (2.41) | 0.42 | 2.76 (2.77) | 0.0052123130 | 0.5985332 |
| 60126 | 1793.88 | 32.3738 | 0.5 | 2.63 (2.59) | 0.78 | 2.51 (2.53) | 0.0063362810 | 0.6108099 |
| 60149 | 1794.8 | 23.9153 | 0.77 | 3.18 (3.21) | 0.96 | 3.27 (3.28) | 0.0027667390 | 0.6242560 |
| 60248 | 1796.77 | 30.9215 | 0.24 | 2.12 (2.06) | 0.52 | 2.19 (2.23) | 0.0000625281 | 0.6454082 |
| 60352 | 1798.72 | 36.9482 | 0.1 | 2.02 (2.01) | 0.5 | 2.30 (2.36) | 0.0000000003 | 0.7112032 |
| 60628 | 1806.83 | 23.0614 | 0.34 | 2.33 (2.37) | 0.78 | 2.30 (2.25) | 0.0000000603 | 0.7151892 |
| 60751 | 1807.81 | 20.6486 | 0.86 | 3.11 (3.06) | 0.97 | 3.35 (3.40) | 0.0000021977 | 0.6967474 |
| 60816 | 1808.79 | 23.7176 | 0.4 | 2.49 (2.53) | 0.66 | 2.42 (2.45) | 0.0047687850 | 0.6109162 |
| 60975 | 1812.79 | 24.1374 | 0.35 | 2.34 (2.43) | 0.2 | 2.35 (2.38) | 0.0234374300 | 0.5739796 |
| 61192 | 1816.9 | 33.7527 | 0.14 | 1.91 (1.89) | 0.3 | 2.17 (2.18) | 0.0035993240 | 0.5880102 |
| 61221 | 1817.69 | 20.2344 | 0.77 | 3.36 (3.37) | 0.97 | 3.53 (3.56) | 0.0000005483 | 0.7079613 |
| 61304 | 1818.83 | 30.9531 | 0.63 | 3.17 (3.42) | 0.53 | 2.32 (2.16) | 0.0001143746 | 0.6544430 |
| 61332 | 1819.8 | 23.3631 | 0.95 | 3.61 (3.61) | 0.99 | 3.74 (3.74) | 0.0006954562 | 0.6409970 |
| 61340 | 1819.83 | 32.1253 | 0.32 | 2.54 (2.58) | 0.13 | 1.73 (1.71) | 0.0002731040 | 0.6100128 |
| 61404 | 1821.83 | 31.7802 | 0.11 | 2.34 (2.16) | 0.31 | 2.03 (2.06) | 0.0013005090 | 0.5954507 |
| 61405 | 1821.82 | 30.1713 | 0.34 | 2.43 (2.39) | 0.63 | 2.50 (2.52) | 0.0000671091 | 0.6535927 |
| 61573 | 1825.79 | 20.135 | 0.73 | 3.26 (3.33) | 0.94 | 3.38 (3.44) | 0.0002581935 | 0.6515731 |
| 61576 | 1825.8 | 31.931 | 0.32 | 2.31 (2.35) | 0.61 | 2.37 (2.40) | 0.0000508976 | 0.6549213 |
| 61711 | 1828.85 | 21.202 | 0.56 | 2.77 (2.81) | 0.28 | 2.17 (2.14) | 0.0000001359 | 0.6969069 |
| 61945 | 1834.83 | 31.095 | 0.78 | 3.53 (3.65) | 0.77 | 3.02 (3.03) | 0.0005966551 | 0.6419005 |
| 61984 | 1835.71 | 19.9131 | 0.43 | 2.71 (2.81) | 0.85 | 2.99 (3.07) | 0.0000000000 | 0.7767857 |
| 62000 | 1835.84 | 24.0379 | 0.55 | 2.51 (2.48) | 0.33 | 2.20 (2.33) | 0.0001234493 | 0.6451956 |
| 62080 | 1837.8 | 30.5569 | 0.14 | 2.52 (2.21) | 0.35 | 2.39 (2.41) | 0.0005988675 | 0.6080995 |
| 62256 | 1841.75 | 35.6598 | 0.21 | 1.96 (1.95) | 0.43 | 1.97 (1.99) | 0.0018021470 | 0.6074086 |
| 62323 | 1842.85 | 24.0672 | 0.1 | 2.58 (2.52) | 0.34 | 2.60 (2.58) | 0.0000465518 | 0.6230867 |
| 62547 | 1847.89 | 43.6655 | 0.15 | 1.96 (1.88) | 0.38 | 2.35 (2.50) | 0.0001090014 | 0.6245748 |
| 62580 | 1848.81 | 30.8099 | 0.42 | 2.07 (2.01) | 0.67 | 1.96 (1.92) | 0.0086014100 | 0.6038478 |
| 62866 | 1854.82 | 40.9238 | 0.91 | 3.64 (3.74) | 0.9 | 3.78 (3.82) | 0.0043823251 | 0.6160980 |
| 63098 | 1858.84 | 24.2646 | 0.19 | 2.35 (2.37) | 0.43 | 2.60 (2.56) | 0.0001632402 | 0.6283482 |
| 63135 | 1859.8 | 31.3884 | 0.18 | 2.30 (2.45) | 0.47 | 2.43 (2.45) | 0.0000164336 | 0.6490221 |
| 63143 | 1859.83 | 24.4114 | 0.29 | 2.57 (2.67) | 0.5 | 2.36 (2.46) | 0.0226583200 | 0.5834396 |
| 63209 | 1860.83 | 21.4001 | 0.44 | 2.91 (2.98) | 0.9 | 2.91 (2.95) | 0.0000001363 | 0.7149235 |
| 63427 | 1865.81 | 32.982 | 0.24 | 1.99 (1.99) | 0.46 | 1.95 (1.97) | 0.0043723600 | 0.6009779 |
| 63517 | 1867.95 | 25.1463 | 0.35 | 2.49 (2.31) | 0.2 | 2.20 (2.18) | 0.0093608510 | 0.5848214 |
| 63812 | 1874.83 | 30.8238 | 0.08 | 2.02 (1.94) | 0.43 | 2.15 (2.10) | 0.0000000268 | 0.6764456 |
| 63910 | 1876.87 | 22.2013 | 0.9 | 3.33 (3.37) | 0.99 | 3.11 (3.13) | 0.0055385790 | 0.6153274 |
| 64054 | 1878.79 | 20.7224 | 0.39 | 2.66 (2.70) | 0.81 | 2.71 (2.73) | 0.0000000289 | 0.7230017 |
| 64067 | 1878.85 | 32.1809 | 0.15 | 1.91 (1.81) | 0.32 | 1.92 (2.07) | 0.0066665240 | 0.5841305 |
| 64170 | 1880.9 | 43.9095 | 0.3 | 2.48 (2.53) | 0.6 | 2.79 (2.88) | 0.0000005731 | 0.6896259 |
| 64431 | 1885.65 | 38.82 | 0.31 | 2.32 (2.28) | 0.69 | 2.28 (2.32) | 0.0000031235 | 0.6809099 |
| 64493 | 1886.86 | 27.2961 | 0.28 | 2.10 (2.15) | 0.46 | 2.02 (2.03) | 0.0209151500 | 0.5829082 |
| 64869 | 1892.77 | 40.25 | 0.23 | 2.79 (2.67) | 0.39 | 2.87 (3.02) | 0.0209861300 | 0.5786033 |
| 64899 | 1892.97 | 24.5581 | 0.49 | 2.59 (2.65) | 0.64 | 2.72 (2.78) | 0.0065447180 | 0.6082058 |
| 65035 | 1895.07 | 21.396 | 0.44 | 2.70 (2.73) | 0.21 | 2.53 (2.63) | 0.0003644468 | 0.6232993 |
| 65257 | 1899.85 | 24.8347 | 0.43 | 2.26 (2.22) | 0.26 | 1.84 (1.81) | 0.0006973302 | 0.6195791 |
| 65312 | 1900.87 | 31.9971 | 0.18 | 2.10 (2.15) | 0.43 | 2.10 (2.14) | 0.0003246692 | 0.6217049 |
| 65368 | 1901.82 | 43.8255 | 0.38 | 2.74 (2.82) | 0.49 | 2.89 (2.96) | 0.0001677757 | 0.6290391 |
| 65593 | 1907.77 | 23.7801 | 0.1 | 1.91 (1.91) | 0.38 | 1.93 (1.94) | 0.0000108821 | 0.6363733 |
| 66054 | 1915 | 33.6261 | 0.38 | 2.14 (2.21) | 0.24 | 1.92 (1.92) | 0.0171630100 | 0.5811543 |
| 66161 | 1916.77 | 20.3224 | 0.5 | 2.93 (2.98) | 0.82 | 3.19 (3.29) | 0.0000000134 | 0.7314520 |
| 66197 | 1916.95 | 34.193 | 0.35 | 2.07 (2.10) | 0.6 | 2.06 (2.09) | 0.0006637182 | 0.6308461 |
| 67217 | 1933.88 | 21.6245 | 0.87 | 2.90 (2.91) | 0.93 | 2.59 (2.52) | 0.0016180740 | 0.6310055 |
| 67263 | 1934.79 | 19.9423 | 0.27 | 2.50 (2.52) | 0.74 | 2.79 (2.79) | 0.0000000000 | 0.7729592 |
| 67306 | 1935.81 | 20.6619 | 0.23 | 2.38 (2.46) | 0.53 | 2.45 (2.57) | 0.0000302761 | 0.6515200 |
| 67462 | 1938.88 | 21.3856 | 0.21 | 2.46 (2.50) | 0.44 | 1.98 (2.01) | 0.0150912400 | 0.5840774 |
| 67612 | 1942.84 | 30.9624 | 0.32 | 1.97 (2.03) | 0.52 | 2.02 (2.04) | 0.0038419140 | 0.6076743 |
| 67632 | 1943.01 | 24.9416 | 0.32 | 3.56 (3.70) | 0.05 | 2.89 (2.54) | 0.0000016984 | 0.6349384 |
| 67723 | 1945.88 | 41.9013 | 0.33 | 2.76 (2.78) | 0.07 | 2.27 (2.29) | 0.0000036700 | 0.6347258 |
| 67801 | 1947.88 | 31.6062 | 0.45 | 2.51 (2.55) | 0.64 | 2.56 (2.61) | 0.0067097680 | 0.6071429 |
| 67951 | 1950.85 | 35.7688 | 0.78 | 2.66 (2.71) | 0.88 | 2.79 (2.82) | 0.0011678980 | 0.6346195 |
| 68081 | 1954.01 | 32.4121 | 0.11 | 2.61 (2.59) | 0.32 | 2.12 (2.17) | 0.0013475700 | 0.5960353 |
| 68117 | 1954.97 | 25.3585 | 0.47 | 2.48 (2.41) | 0.32 | 2.28 (2.35) | 0.0154620100 | 0.5889668 |
| 68411 | 1962.83 | 21.9701 | 0.23 | 2.54 (2.54) | 0.56 | 2.41 (2.39) | 0.0000301098 | 0.6532738 |
| 68663 | 1968.88 | 25.1998 | 0.47 | 2.62 (2.63) | 0.21 | 2.58 (2.73) | 0.0002102432 | 0.6301020 |
| 69080 | 1976.88 | 32.3847 | 0.52 | 2.51 (2.55) | 0.69 | 2.54 (2.60) | 0.0181383600 | 0.5951318 |
| 69681 | 1989.88 | 32.4375 | 0.68 | 2.34 (2.39) | 0.82 | 2.40 (2.41) | 0.0084426710 | 0.6086841 |
| 69882 | 1993.88 | 32.1852 | 0.74 | 2.34 (2.39) | 0.74 | 2.09 (2.12) | 0.0194273000 | 0.5963542 |
| 69979 | 1996.79 | 20.9757 | 0.39 | 2.74 (2.72) | 0.86 | 2.99 (3.02) | 0.0000000000 | 0.7988946 |
| 70456 | 2008.9 | 32.2865 | 0.16 | 2.62 (2.67) | 0.47 | 2.72 (2.69) | 0.0000033366 | 0.6591199 |
| 70485 | 2009.88 | 32.2918 | 0.77 | 2.81 (2.84) | 0.55 | 2.49 (2.65) | 0.0000005981 | 0.7033907 |
| 70633 | 2013.89 | 31.7558 | 0.32 | 1.97 (1.94) | 0.54 | 1.96 (2.03) | 0.0024808810 | 0.6134141 |
| 70635 | 2013.91 | 25.1946 | 0.97 | 3.20 (3.32) | 0.96 | 3.15 (3.19) | 0.0044721130 | 0.6181973 |
| 70803 | 2017.88 | 30.7718 | 0.14 | 2.27 (2.37) | 0.32 | 2.32 (2.30) | 0.0031615550 | 0.5907738 |
| 71171 | 2023.91 | 21.4813 | 0.7 | 2.53 (2.53) | 0.66 | 2.30 (2.28) | 0.0091036900 | 0.6066645 |
| 71312 | 2025.87 | 32.2255 | 0.94 | 2.91 (2.95) | 0.92 | 2.76 (2.79) | 0.0001492695 | 0.6576318 |
| 71490 | 2029.85 | 20.3904 | 0.31 | 2.54 (2.50) | 0.67 | 2.43 (2.48) | 0.0000206707 | 0.6644345 |
| 71602 | 2030.93 | 32.6077 | 0.91 | 3.17 (3.21) | 0.91 | 2.92 (2.96) | 0.0000223965 | 0.6761798 |
| 71943 | 2034.88 | 19.5206 | 0.09 | 2.11 (2.05) | 0.36 | 2.39 (2.43) | 0.0000034504 | 0.6415285 |
| 72033 | 2034.94 | 25.4531 | 0.35 | 2.70 (2.63) | 0.23 | 2.18 (2.26) | 0.0130539600 | 0.5825893 |
| 72048 | 2035 | 40.1854 | 0.31 | 1.88 (1.85) | 0.52 | 2.05 (2.09) | 0.0004328481 | 0.6306335 |
| 72343 | 2042.07 | 25.1431 | 0.43 | 3.28 (3.26) | 0.03 | 2.49 (2.42) | 0.0000000001 | 0.7011054 |
| 72483 | 2045.86 | 34.1702 | 0.29 | 2.48 (2.54) | 0.49 | 2.44 (2.43) | 0.0084939200 | 0.5959821 |
| 72533 | 2046.92 | 32.5791 | 0.94 | 3.38 (3.46) | 0.91 | 3.17 (3.17) | 0.0000003894 | 0.7108844 |
| 72641 | 2048.93 | 24.4632 | 0.98 | 3.44 (3.48) | 0.96 | 3.00 (3.03) | 0.0000000000 | 0.7834821 |
| 72896 | 2055.94 | 25.438 | 0.92 | 3.06 (3.06) | 0.96 | 2.85 (2.88) | 0.0000098131 | 0.6837798 |
| 73010 | 2058.94 | 23.1508 | 0.4 | 2.24 (2.23) | 0.71 | 2.36 (2.47) | 0.0000034391 | 0.6840986 |
| 73015 | 2059.01 | 33.0804 | 0.51 | 2.41 (2.42) | 0.23 | 1.85 (1.87) | 0.0000006583 | 0.6792092 |
| 73177 | 2062.93 | 26.579 | 0.87 | 2.89 (2.91) | 0.92 | 2.66 (2.72) | 0.0220688800 | 0.5951318 |
| 73291 | 2064.92 | 24.4599 | 0.86 | 2.94 (3.04) | 0.73 | 2.48 (2.55) | 0.0000000243 | 0.7308673 |
| 73434 | 2067.82 | 20.6208 | 0.51 | 2.92 (2.91) | 0.93 | 3.17 (3.27) | 0.0000000000 | 0.8159014 |
| 73913 | 2076.95 | 21.7789 | 0.27 | 2.61 (2.61) | 0.6 | 2.73 (2.81) | 0.0000010298 | 0.6836735 |
| 73989 | 2077.96 | 22.4848 | 0.17 | 2.27 (2.26) | 0.36 | 2.18 (2.21) | 0.0059187590 | 0.5892326 |
| 74057 | 2079 | 24.6778 | 0.47 | 2.38 (2.35) | 0.72 | 2.34 (2.34) | 0.0048518570 | 0.6130952 |
| 74065 | 2078.93 | 26.6715 | 0.99 | 3.88 (3.93) | 1 | 3.82 (3.84) | 0.0128745600 | 0.6034226 |
| 74187 | 2080.94 | 20.201 | 0.42 | 2.66 (2.66) | 0.71 | 2.80 (2.86) | 0.0000066584 | 0.6792092 |
| 74273 | 2081.94 | 33.6612 | 0.65 | 2.67 (2.74) | 0.73 | 2.25 (2.26) | 0.0129226300 | 0.6018282 |
| 74420 | 2085.93 | 22.0672 | 0.26 | 3.77 (4.11) | 0.45 | 4.17 (4.17) | 0.0020728820 | 0.6091093 |
| 74703 | 2087.84 | 19.4213 | 0.1 | 2.37 (2.49) | 0.42 | 2.54 (2.55) | 0.0000004780 | 0.6609269 |
| 74987 | 2089.96 | 39.5167 | 0.34 | 2.15 (2.12) | 0.57 | 2.24 (2.30) | 0.0001775150 | 0.6425914 |
| 75248 | 2096.92 | 32.9972 | 0.53 | 2.40 (2.42) | 0.36 | 2.21 (2.28) | 0.0041124890 | 0.6088435 |
| 75846 | 2103.96 | 33.0368 | 0.97 | 3.36 (3.38) | 0.98 | 3.16 (3.20) | 0.0000415814 | 0.6703869 |
| 76053 | 2108.93 | 32.932 | 0.07 | 1.96 (2.04) | 0.3 | 1.95 (1.99) | 0.0000380922 | 0.6161246 |
| 76086 | 2109.92 | 24.069 | 0.22 | 2.23 (2.24) | 0.44 | 2.22 (2.23) | 0.0025743920 | 0.6048044 |
| 76090 | 2109.96 | 21.086 | 0.12 | 2.13 (2.18) | 0.41 | 2.15 (2.12) | 0.0000119602 | 0.6409439 |
| 76415 | 2117.93 | 32.9682 | 0.32 | 2.06 (2.06) | 0.49 | 2.08 (2.12) | 0.0122927300 | 0.5923151 |
| 76636 | 2124.84 | 20.3719 | 0.07 | 2.11 (2.23) | 0.36 | 2.22 (2.19) | 0.0000007891 | 0.6479060 |
| 76839 | 2128.98 | 26.9686 | 0.3 | 1.95 (1.89) | 0.48 | 2.05 (2.08) | 0.0033981320 | 0.6068240 |
| 77018 | 2133.96 | 27.7658 | 0.96 | 3.60 (3.63) | 0.95 | 3.35 (3.39) | 0.0000048937 | 0.6899447 |
| 77099 | 2135.96 | 25.8003 | 0.69 | 2.69 (2.69) | 0.59 | 2.51 (2.52) | 0.0115105400 | 0.6026786 |
| 77236 | 2138.95 | 24.235 | 0.36 | 2.30 (2.26) | 0.19 | 1.88 (1.85) | 0.0015829320 | 0.6031037 |
| 77684 | 2148.02 | 26.0398 | 0.35 | 2.82 (2.77) | 0.21 | 2.43 (2.39) | 0.0100189900 | 0.5846088 |
| 77763 | 2149.96 | 27.7553 | 0.97 | 3.42 (3.49) | 0.98 | 3.15 (3.17) | 0.0000000202 | 0.7332058 |
| 78073 | 2156.97 | 22.2173 | 0.45 | 2.68 (2.68) | 0.88 | 3.05 (3.15) | 0.0000000000 | 0.8025085 |
| 78792 | 2168.97 | 32.9057 | 0.81 | 2.78 (2.79) | 0.69 | 2.61 (2.65) | 0.0019486390 | 0.6277636 |
| 79136 | 2175.01 | 33.2806 | 1 | 3.61 (3.65) | 1 | 3.55 (3.57) | 0.0067897495 | 0.6163372 |
| 79720 | 2187.95 | 39.7827 | 0.94 | 2.90 (2.88) | 0.97 | 3.10 (3.17) | 0.0116308600 | 0.6049107 |
| 79786 | 2189 | 26.885 | 0.64 | 2.70 (2.73) | 0.85 | 2.71 (2.75) | 0.0048488900 | 0.6161777 |
| 79918 | 2190.99 | 25.823 | 0.38 | 3.05 (3.11) | 0.17 | 2.99 (3.03) | 0.0006897038 | 0.6107568 |
| 80012 | 2191.99 | 22.3935 | 0.45 | 2.59 (2.66) | 0.74 | 2.70 (2.77) | 0.0000084483 | 0.6787840 |
| 80306 | 2194.97 | 20.1667 | 0.17 | 2.43 (2.49) | 0.5 | 2.57 (2.58) | 0.0000008773 | 0.6717687 |
| 80360 | 2196.02 | 33.1586 | 0.44 | 2.79 (2.79) | 0.35 | 2.48 (2.43) | 0.0000009843 | 0.6522109 |
| 80829 | 2203.64 | 35.2621 | 0.04 | 1.72 (1.71) | 0.3 | 1.92 (1.89) | 0.0000010912 | 0.6326531 |
| 81181 | 2210.76 | 37.0638 | 0.24 | 2.23 (2.26) | 0.44 | 2.18 (2.22) | 0.0081421970 | 0.5928997 |
| 81196 | 2210.95 | 33.6055 | 0.97 | 3.70 (3.79) | 1 | 3.63 (3.64) | 0.0147430200 | 0.6014031 |
| 81272 | 2211.98 | 33.2336 | 0.38 | 2.74 (2.82) | 0.44 | 2.60 (2.74) | 0.0074191674 | 0.6298895 |
| 81457 | 2216.03 | 33.8306 | 0.55 | 2.16 (2.19) | 0.73 | 2.41 (2.48) | 0.0000953148 | 0.6583759 |
| 82015 | 2226.97 | 33.4565 | 0.65 | 2.28 (2.36) | 0.59 | 2.06 (2.04) | 0.0146635300 | 0.5987457 |
| 82026 | 2226.99 | 26.2775 | 0.6 | 3.97 (4.06) | 0.85 | 4.15 (4.15) | 0.0001331921 | 0.6572066 |
| 82708 | 2235.05 | 34.1665 | 0.5 | 2.43 (2.45) | 0.78 | 2.70 (2.75) | 0.0000000740 | 0.7183780 |
| 82784 | 2236.98 | 27.1389 | 0.66 | 2.42 (2.41) | 0.69 | 2.32 (2.30) | 0.0014908720 | 0.6602892 |
| 83081 | 2243.97 | 21.1604 | 0.21 | 2.29 (2.25) | 0.43 | 2.19 (2.29) | 0.0029816700 | 0.6022003 |
| 83441 | 2248.97 | 33.6947 | 0.21 | 2.94 (3.07) | 0.27 | 2.92 (2.92) | 0.0014908381 | 0.6562235 |
| 84164 | 2257.87 | 35.9274 | 0.18 | 2.32 (2.31) | 0.57 | 2.69 (2.71) | 0.0000000062 | 0.7102466 |
| 84300 | 2261.03 | 27.1527 | 0.32 | 2.74 (2.80) | 0.5 | 2.92 (3.04) | 0.0046159070 | 0.6048044 |
| 84484 | 2264.94 | 43.2938 | 0.35 | 2.13 (2.18) | 0.15 | 2.15 (2.19) | 0.0012845350 | 0.6014031 |
| 84704 | 2269.03 | 39.327 | 0.62 | 2.27 (2.28) | 0.51 | 2.04 (2.09) | 0.0078112190 | 0.6060268 |
| 84909 | 2274.04 | 33.5062 | 0.16 | 2.03 (1.94) | 0.31 | 2.05 (2.07) | 0.0149527600 | 0.5754677 |
| 85020 | 2276.02 | 27.2312 | 0.46 | 3.13 (3.24) | 0.81 | 3.39 (3.52) | 0.0000000173 | 0.7285289 |
| 85076 | 2277.01 | 27.2248 | 0.6 | 3.09 (3.32) | 0.44 | 2.83 (2.88) | 0.0008577108 | 0.6307398 |
| 85250 | 2280.94 | 36.2186 | 0.14 | 2.35 (2.37) | 0.34 | 2.22 (2.16) | 0.0024449390 | 0.5947066 |
| 85331 | 2282.02 | 22.2394 | 0.65 | 2.75 (2.81) | 0.89 | 2.74 (2.85) | 0.0033327370 | 0.6212798 |
| 85627 | 2289.04 | 33.5902 | 0.27 | 2.10 (2.04) | 0.46 | 2.17 (2.07) | 0.0043870830 | 0.6018282 |
| 85761 | 2292.02 | 27.2827 | 0.89 | 3.35 (3.46) | 0.99 | 3.52 (3.60) | 0.0018525380 | 0.6294111 |
| 86071 | 2298.99 | 27.0777 | 0.22 | 2.67 (2.74) | 0.4 | 2.84 (2.83) | 0.0056779830 | 0.5941752 |
| 86226 | 2302.9 | 43.1327 | 0.31 | 2.32 (2.45) | 0.11 | 1.91 (2.01) | 0.0004608817 | 0.6039541 |
| 86354 | 2305.06 | 34.9093 | 0.14 | 2.21 (2.29) | 0.34 | 1.97 (1.99) | 0.0029238620 | 0.5930060 |
| 86677 | 2308.02 | 27.3371 | 0.33 | 2.12 (2.06) | 0.56 | 2.19 (2.27) | 0.0006065091 | 0.6296769 |
| 86951 | 2314.01 | 33.6555 | 0.58 | 2.56 (2.59) | 0.8 | 2.65 (2.68) | 0.0005182047 | 0.6421662 |
| 87365 | 2320.07 | 20.7338 | 0.09 | 2.19 (2.06) | 0.39 | 2.30 (2.33) | 0.0000012568 | 0.6502445 |
| 87692 | 2327.91 | 21.0034 | 0.15 | 2.26 (2.26) | 0.58 | 2.42 (2.46) | 0.0000000003 | 0.7264031 |
| 88093 | 2336.04 | 26.6568 | 0.43 | 2.18 (2.23) | 0.21 | 1.86 (1.80) | 0.0001928141 | 0.6282951 |
| 88282 | 2339 | 34.0072 | 0.56 | 2.59 (2.62) | 0.84 | 2.79 (2.80) | 0.0000003184 | 0.7096620 |
| 88972 | 2349.04 | 27.3655 | 0.22 | 2.05 (2.01) | 0.4 | 2.11 (2.11) | 0.0071938720 | 0.5915179 |
| 89083 | 2352.05 | 26.7539 | 0.6 | 2.73 (2.78) | 0.34 | 2.27 (2.28) | 0.0000006841 | 0.6908482 |
| 89604 | 2360 | 33.9228 | 0.17 | 1.80 (1.72) | 0.32 | 1.89 (1.88) | 0.0124703300 | 0.5787096 |
| 89642 | 2361.11 | 20.7943 | 0.3 | 2.26 (2.27) | 0.48 | 2.59 (2.58) | 0.0007854071 | 0.6224490 |
| 89900 | 2368.04 | 26.7503 | 0.65 | 2.55 (2.63) | 0.45 | 2.11 (2.15) | 0.0000009711 | 0.6941964 |
| 90344 | 2377.1 | 20.7997 | 0.68 | 2.98 (3.07) | 0.93 | 3.18 (3.30) | 0.0000006430 | 0.7061543 |
| 90470 | 2380.08 | 36.5109 | 0.21 | 2.16 (2.22) | 0.43 | 2.19 (2.15) | 0.0024092340 | 0.6044324 |
| 90840 | 2389.24 | 22.3992 | 0.43 | 3.26 (3.22) | 0.08 | 2.48 (2.58) | 0.0000000148 | 0.6810162 |
| 91342 | 2403.19 | 24.9373 | 0.45 | 2.43 (2.49) | 0.15 | 2.22 (2.22) | 0.0000005576 | 0.6684736 |
| 91375 | 2404.02 | 20.2728 | 0.65 | 2.61 (2.63) | 0.81 | 2.70 (2.77) | 0.0059993770 | 0.6131484 |
| 91542 | 2407.09 | 27.6715 | 0.68 | 2.51 (2.54) | 0.92 | 2.61 (2.71) | 0.0000136818 | 0.6800595 |
| 91669 | 2411.08 | 26.8029 | 0.31 | 2.08 (2.09) | 0.17 | 1.85 (1.84) | 0.0121080600 | 0.5778061 |
| 92029 | 2414.63 | 35.6167 | 0.77 | 2.69 (2.78) | 0.56 | 2.55 (2.61) | 0.0007966420 | 0.6367985 |
| 92231 | 2421 | 34.8608 | 0.18 | 1.89 (1.88) | 0.44 | 1.99 (2.04) | 0.0000988539 | 0.6325468 |
| 92410 | 2423.09 | 27.666 | 0.62 | 2.26 (2.29) | 0.83 | 2.34 (2.37) | 0.0010850230 | 0.6344600 |
| 92841 | 2430.1 | 28.3281 | 0.65 | 2.42 (2.41) | 0.84 | 2.47 (2.52) | 0.0023968450 | 0.6252126 |
| 92871 | 2430.59 | 35.5423 | 0.73 | 2.85 (2.93) | 0.51 | 2.75 (2.80) | 0.0010127200 | 0.6329719 |
| 93227 | 2442.07 | 34.0967 | 0.79 | 2.68 (2.71) | 0.94 | 2.81 (2.91) | 0.0000546349 | 0.6675170 |
| 93361 | 2445.1 | 28.2378 | 0.21 | 2.07 (2.05) | 0.39 | 2.09 (2.03) | 0.0108932800 | 0.5857249 |
| 93417 | 2446.09 | 28.3726 | 0.33 | 2.03 (2.00) | 0.49 | 2.14 (2.17) | 0.0075463770 | 0.5988520 |
| 95084 | 2490.23 | 24.6804 | 0.62 | 2.36 (2.43) | 0.48 | 2.18 (2.24) | 0.0016598690 | 0.6247343 |
| 96716 | 2525.2 | 27.7364 | 0.49 | 2.53 (2.48) | 0.63 | 2.82 (2.87) | 0.0007263183 | 0.6342474 |
| 96879 | 2529.17 | 26.992 | 0.24 | 2.03 (2.13) | 0.43 | 1.98 (1.98) | 0.0166664200 | 0.5836522 |
| 97349 | 2541.19 | 27.8949 | 0.13 | 2.06 (1.90) | 0.36 | 1.90 (1.90) | 0.0003404815 | 0.6128295 |
| 97463 | 2544.13 | 28.2599 | 0.22 | 2.05 (2.01) | 0.41 | 2.14 (2.27) | 0.0053525990 | 0.5953444 |
| 97506 | 2545.12 | 28.2016 | 0.27 | 2.27 (2.27) | 0.42 | 2.28 (2.35) | 0.0237350300 | 0.5794005 |
| 97599 | 2547.99 | 21.4417 | 0.38 | 2.37 (2.37) | 0.72 | 2.62 (2.68) | 0.0000000270 | 0.7200787 |
| 97736 | 2551.15 | 34.7151 | 0.67 | 2.54 (2.62) | 0.57 | 2.16 (2.16) | 0.0009664643 | 0.6335034 |
| 97965 | 2557.17 | 28.2473 | 0.29 | 2.10 (2.13) | 0.51 | 2.12 (2.14) | 0.0012549610 | 0.6185162 |
| 98089 | 2559.18 | 19.4074 | 0.47 | 3.14 (3.06) | 0.52 | 2.92 (2.98) | 0.0006891154 | 0.6287203 |
| 98485 | 2560.12 | 34.1541 | 0.23 | 2.00 (2.04) | 0.52 | 1.93 (1.91) | 0.0001232697 | 0.6389243 |
| 98596 | 2563.15 | 21.2101 | 0.64 | 2.85 (2.94) | 0.89 | 3.05 (3.08) | 0.0000062978 | 0.6864902 |
| 98720 | 2565.14 | 23.7401 | 0.44 | 2.40 (2.42) | 0.34 | 1.96 (1.92) | 0.0193507500 | 0.5856186 |
| 98891 | 2567.13 | 34.802 | 0.71 | 2.29 (2.34) | 0.54 | 1.99 (1.97) | 0.0000373327 | 0.6669855 |
| 99021 | 2570.19 | 42.5602 | 0.94 | 3.75 (3.85) | 0.96 | 3.81 (3.88) | 0.0005250289 | 0.6391369 |
| 99251 | 2574.01 | 32.8084 | 0.19 | 1.81 (1.77) | 0.42 | 1.81 (1.82) | 0.0010127250 | 0.6112883 |
| 99475 | 2577.25 | 24.6659 | 0.87 | 2.76 (2.80) | 0.9 | 2.43 (2.49) | 0.0000009559 | 0.7035502 |
| 99691 | 2582.17 | 23.67 | 0.47 | 2.33 (2.28) | 0.35 | 2.08 (2.02) | 0.0184649400 | 0.5874787 |
| 99808 | 2584.23 | 35.1828 | 0.67 | 2.42 (2.40) | 0.9 | 2.71 (2.79) | 0.0000000640 | 0.7236395 |
| 99919 | 2587.2 | 21.1 | 0.18 | 2.46 (2.66) | 0.47 | 2.63 (2.73) | 0.0000196726 | 0.6476403 |
| 100020 | 2589.06 | 22.5622 | 0.19 | 1.98 (1.96) | 0.43 | 2.07 (2.13) | 0.0003053540 | 0.6229273 |
| 100344 | 2599.19 | 28.2751 | 0.73 | 2.28 (2.29) | 0.61 | 2.12 (2.10) | 0.0055995190 | 0.6132015 |
| 101157 | 2616.16 | 28.3947 | 0.09 | 1.96 (2.06) | 0.4 | 1.87 (1.85) | 0.0000019491 | 0.6487032 |
| 101786 | 2627.23 | 34.9933 | 0.14 | 2.56 (2.67) | 0.39 | 2.31 (2.38) | 0.0006102720 | 0.6103316 |
| 101839 | 2628.22 | 34.9664 | 0.86 | 2.87 (2.96) | 0.73 | 2.62 (2.62) | 0.0000017590 | 0.6978104 |
| 102269 | 2638.18 | 28.4204 | 0.07 | 2.06 (2.31) | 0.18 | 2.33 (2.35) | 0.0003400506 | 0.6627870 |
| 102371 | 2639.29 | 21.4189 | 0.33 | 2.27 (2.34) | 0.54 | 2.45 (2.50) | 0.0006783421 | 0.6277636 |
| 102725 | 2642.21 | 27.696 | 0.73 | 2.47 (2.51) | 0.88 | 2.61 (2.65) | 0.0001790725 | 0.6551871 |
| 102819 | 2644.22 | 21.1526 | 0.3 | 2.49 (2.54) | 0.54 | 2.70 (2.71) | 0.0000660460 | 0.6485969 |
| 103022 | 2649.2 | 34.8471 | 0.14 | 2.54 (2.61) | 0.39 | 2.30 (2.29) | 0.0004544118 | 0.6128827 |
| 103198 | 2654.19 | 23.9249 | 0.79 | 2.57 (2.62) | 0.84 | 2.37 (2.36) | 0.0002639798 | 0.6219707 |
| 103224 | 2655.14 | 22.3309 | 0.15 | 2.03 (2.01) | 0.42 | 2.02 (1.93) | 0.0000735479 | 0.6310587 |
| 103493 | 2658.22 | 19.5022 | 0.11 | 3.95 (4.03) | 0.28 | 3.24 (3.21) | 0.0247725150 | 0.5930591 |
| 104195 | 2663.2 | 23.5069 | 0.84 | 2.82 (2.86) | 0.79 | 2.69 (2.84) | 0.0245079250 | 0.5761586 |
| 104786 | 2679.2 | 23.5289 | 0.97 | 3.46 (3.51) | 0.91 | 3.23 (3.32) | 0.0000013531 | 0.7008397 |
| 104954 | 2682.14 | 22.4918 | 0.81 | 2.81 (2.84) | 0.99 | 3.00 (3.01) | 0.0000005881 | 0.7075361 |
| 105105 | 2687.22 | 28.9855 | 0.13 | 1.67 (1.76) | 0.36 | 1.94 (1.94) | 0.0000716723 | 0.6250531 |
| 105352 | 2695.2 | 23.5225 | 0.97 | 3.60 (3.66) | 0.96 | 3.36 (3.37) | 0.0000045503 | 0.6905825 |
| 105661 | 2702.21 | 38.0799 | 0.7 | 2.58 (2.59) | 0.52 | 2.31 (2.33) | 0.0005128093 | 0.6401998 |
| 106067 | 2713.23 | 29.2227 | 0.22 | 2.05 (2.08) | 0.48 | 2.08 (2.09) | 0.0002319599 | 0.6304209 |
| 106195 | 2716.37 | 20.1907 | 0.34 | 3.42 (3.27) | 0.04 | 2.63 (2.68) | 0.0000001261 | 0.6506165 |
| 106678 | 2726.25 | 28.9168 | 0.23 | 2.02 (1.98) | 0.48 | 2.19 (2.23) | 0.0000737277 | 0.6410502 |
| 107198 | 2736.4 | 21.2973 | 0.31 | 3.39 (3.42) | 0.02 | 3.11 (3.11) | 0.0000001081 | 0.6427509 |
| 107460 | 2742.25 | 28.9803 | 0.86 | 2.83 (2.92) | 0.96 | 3.07 (3.14) | 0.0000001832 | 0.7167304 |
| 107858 | 2751.34 | 29.2308 | 0.48 | 2.37 (2.46) | 0.67 | 2.55 (2.57) | 0.0006530693 | 0.6360544 |
| 108021 | 2754.27 | 29.6794 | 0.67 | 2.35 (2.41) | 0.79 | 2.57 (2.59) | 0.0002836969 | 0.6494473 |
| 108574 | 2764.21 | 42.6266 | 0.45 | 3.12 (3.33) | 0.28 | 3.36 (3.61) | 0.0001764960 | 0.6477998 |
| 108724 | 2767.32 | 21.6729 | 0.29 | 2.07 (2.06) | 0.51 | 2.41 (2.41) | 0.0000692951 | 0.6461522 |
| 110052 | 2799.09 | 25.0694 | 0.98 | 3.65 (3.69) | 1 | 3.57 (3.56) | 0.0077877700 | 0.6106505 |
| 110657 | 2816.33 | 28.3966 | 0.18 | 2.13 (2.01) | 0.39 | 1.95 (1.84) | 0.0048116000 | 0.5932185 |
| 110913 | 2823.33 | 29.1158 | 0.5 | 2.47 (2.48) | 0.75 | 2.67 (2.75) | 0.0000018233 | 0.6930272 |
| 111304 | 2834.19 | 22.4673 | 0.23 | 2.18 (2.25) | 0.51 | 2.14 (2.19) | 0.0001410538 | 0.6371705 |
| 111426 | 2837.36 | 23.8684 | 0.55 | 3.28 (3.37) | 0.25 | 3.07 (3.36) | 0.0000216434 | 0.6565689 |
| 111564 | 2841.26 | 24.5354 | 0.68 | 3.06 (3.12) | 0.73 | 2.69 (2.69) | 0.0230105500 | 0.5933248 |
| 112106 | 2854.36 | 34.8636 | 0.94 | 3.31 (3.45) | 1 | 3.56 (3.56) | 0.0012203850 | 0.6344600 |
| 112515 | 2864.25 | 20.1472 | 0.19 | 2.52 (2.63) | 0.38 | 2.74 (2.71) | 0.0018374910 | 0.6029974 |
| 113439 | 2889.38 | 28.6515 | 0.18 | 2.02 (2.01) | 0.36 | 2.04 (2.09) | 0.0045154700 | 0.5926871 |
| 113452 | 2889.35 | 24.0847 | 0.37 | 2.39 (2.40) | 0.47 | 2.36 (2.36) | 0.0038987190 | 0.5984269 |
| 113765 | 2898.31 | 29.2496 | 0.24 | 2.10 (2.07) | 0.46 | 2.06 (2.07) | 0.0032819680 | 0.6041667 |
| 114086 | 2907.35 | 35.9551 | 0.71 | 2.30 (2.31) | 0.59 | 2.14 (2.20) | 0.0098267530 | 0.6051233 |
| 114438 | 2917.37 | 29.038 | 0.61 | 2.90 (2.94) | 0.47 | 2.76 (2.62) | 0.0130572800 | 0.5981080 |
| 114702 | 2923.43 | 36.9151 | 0.26 | 2.13 (2.24) | 0.57 | 2.29 (2.30) | 0.0000019262 | 0.6767113 |
| 114823 | 2926.3 | 22.2183 | 0.41 | 2.56 (2.46) | 0.64 | 2.53 (2.54) | 0.0031104710 | 0.6159651 |
| 115050 | 2932.32 | 34.1472 | 0.36 | 2.29 (2.38) | 0.68 | 2.27 (2.32) | 0.0000826656 | 0.6540710 |
| 115312 | 2939.15 | 33.7671 | 0.17 | 2.03 (2.01) | 0.57 | 2.33 (2.34) | 0.0000000011 | 0.7196535 |
| 116812 | 2977.18 | 19.5232 | 0.35 | 2.68 (2.59) | 0.72 | 2.64 (2.75) | 0.0000016703 | 0.6885629 |
| 117009 | 2977.37 | 29.118 | 0.37 | 2.17 (2.23) | 0.47 | 2.35 (2.38) | 0.0240074000 | 0.5840774 |
| 117317 | 2987.35 | 38.5457 | 0.56 | 2.57 (2.60) | 0.35 | 2.25 (2.32) | 0.0002342502 | 0.6403061 |
| 117524 | 2994.38 | 29.6026 | 0.17 | 1.96 (1.94) | 0.36 | 2.19 (2.25) | 0.0007819083 | 0.6088967 |
| 117823 | 3002.24 | 23.8009 | 0.19 | 1.90 (1.95) | 0.51 | 1.87 (1.88) | 0.0000127561 | 0.6546025 |
| 118053 | 3007.41 | 20.9513 | 0.21 | 2.57 (2.55) | 0.4 | 2.48 (2.55) | 0.0095191090 | 0.5877976 |
| 118163 | 3011.39 | 29.7495 | 0.89 | 2.99 (3.03) | 0.94 | 3.10 (3.17) | 0.0003297513 | 0.6492347 |
| 118224 | 3013.29 | 22.2946 | 0.64 | 3.34 (3.53) | 0.91 | 3.65 (3.75) | 0.0000003192 | 0.7112564 |
| 118597 | 3021.35 | 23.4158 | 0.78 | 2.98 (3.03) | 0.95 | 3.22 (3.27) | 0.0000028299 | 0.6944090 |
| 119292 | 3035.19 | 42.0209 | 0.5 | 2.54 (2.58) | 0.68 | 2.61 (2.69) | 0.0028228080 | 0.6197385 |
| 119660 | 3044.42 | 34.2873 | 0.05 | 1.62 (1.72) | 0.32 | 2.24 (2.34) | 0.0000005474 | 0.6412096 |
| 120192 | 3058.38 | 24.8236 | 0.55 | 2.34 (2.34) | 0.68 | 2.44 (2.56) | 0.0176618900 | 0.5957696 |
| 120423 | 3064.32 | 20.5742 | 0.28 | 2.50 (2.54) | 0.56 | 2.78 (2.86) | 0.0000022183 | 0.6762330 |
| 121070 | 3076.23 | 19.5773 | 0.45 | 2.82 (2.88) | 0.75 | 2.85 (2.85) | 0.0001404418 | 0.6530612 |
| 121413 | 3080.41 | 29.9804 | 0.19 | 2.34 (2.45) | 0.41 | 2.27 (2.48) | 0.0017078800 | 0.6056016 |
| 121716 | 3091.44 | 28.3964 | 0.44 | 2.46 (2.48) | 0.76 | 2.76 (2.81) | 0.0000000212 | 0.7251807 |
| 121775 | 3092.46 | 31.2493 | 0.64 | 2.39 (2.41) | 0.76 | 2.58 (2.68) | 0.0005814212 | 0.6411033 |
| 121940 | 3098.44 | 30.0554 | 0.71 | 2.56 (2.59) | 0.89 | 2.68 (2.75) | 0.0000737948 | 0.6641156 |
| 121989 | 3100.42 | 29.9643 | 0.46 | 2.61 (2.68) | 0.25 | 2.48 (2.58) | 0.0012301270 | 0.6150085 |
| 122216 | 3106.19 | 19.5811 | 0.08 | 2.52 (2.58) | 0.36 | 2.35 (2.42) | 0.0000058103 | 0.6370111 |
| 122400 | 3108.45 | 31.284 | 0.64 | 2.30 (2.30) | 0.86 | 2.46 (2.52) | 0.0000040671 | 0.6901042 |
| 122623 | 3114.43 | 30.2902 | 0.19 | 2.09 (2.09) | 0.35 | 2.31 (2.38) | 0.0038697910 | 0.5942815 |
| 123106 | 3130.43 | 30.8219 | 0.01 | 1.98 (1.98) | 0.09 | 2.63 (2.67) | 0.0021531815 | 0.6187288 |
| 123287 | 3137.41 | 30.3462 | 0.7 | 2.64 (2.70) | 0.6 | 2.42 (2.48) | 0.0004365719 | 0.6431760 |
| 123634 | 3148.28 | 24.1552 | 0.34 | 2.25 (2.16) | 0.64 | 2.30 (2.34) | 0.0000338300 | 0.6601297 |
| 123750 | 3152.34 | 24.5511 | 0.81 | 2.76 (2.80) | 0.67 | 2.62 (2.61) | 0.0015746870 | 0.6302083 |
| 123969 | 3158.44 | 29.7137 | 0.86 | 3.14 (3.14) | 0.74 | 2.77 (2.83) | 0.0000001231 | 0.7189626 |
| 124193 | 3166.27 | 22.0589 | 0.44 | 2.61 (2.61) | 0.71 | 2.89 (2.93) | 0.0000021314 | 0.6892007 |
| 124735 | 3187.36 | 35.6177 | 0.32 | 2.19 (2.15) | 0.53 | 2.40 (2.46) | 0.0002818371 | 0.6356824 |
| 124886 | 3193.38 | 22.6436 | 0.46 | 2.85 (2.89) | 0.74 | 2.89 (3.02) | 0.0002622249 | 0.6467368 |
| 124909 | 3194.42 | 30.3977 | 0.55 | 2.62 (2.59) | 0.17 | 2.13 (2.01) | 0.0000000009 | 0.7186437 |
| 125103 | 3202.43 | 30.6017 | 0.16 | 2.03 (2.10) | 0.44 | 2.23 (2.32) | 0.0000139200 | 0.6462585 |
| 125263 | 3205.27 | 19.6576 | 0.67 | 2.87 (2.85) | 0.84 | 3.02 (3.06) | 0.0001650289 | 0.6555060 |
| 125749 | 3223.31 | 21.6127 | 0.18 | 2.24 (2.26) | 0.55 | 2.45 (2.47) | 0.0000000392 | 0.6971726 |
| 125799 | 3223.42 | 39.1265 | 0.64 | 2.98 (3.09) | 0.86 | 3.19 (3.29) | 0.0000830968 | 0.6623618 |
| 126699 | 3248.53 | 30.6604 | 0.77 | 3.00 (3.03) | 0.77 | 3.26 (3.28) | 0.0027494100 | 0.6237245 |
| 126982 | 3256.53 | 33.0342 | 0.38 | 2.72 (2.83) | 0.63 | 2.95 (3.01) | 0.0001359242 | 0.6483844 |
| 127106 | 3260.49 | 41.6176 | 0.22 | 2.41 (2.43) | 0.4 | 2.59 (2.73) | 0.0036200830 | 0.5990646 |
| 127237 | 3261.5 | 22.1948 | 0.14 | 2.27 (2.23) | 0.31 | 2.30 (2.32) | 0.0062185800 | 0.5834396 |
| 127354 | 3264.53 | 30.6582 | 0.51 | 2.33 (2.39) | 0.69 | 2.37 (2.43) | 0.0198660400 | 0.5936437 |
| 127443 | 3266.48 | 30.0713 | 0.14 | 1.96 (1.97) | 0.31 | 1.87 (1.87) | 0.0102103600 | 0.5783376 |
| 127575 | 3271.49 | 30.7045 | 0.44 | 2.95 (3.03) | 0.28 | 2.79 (2.92) | 0.0138698900 | 0.5879571 |
| 127731 | 3276.24 | 33.377 | 0.17 | 1.78 (1.74) | 0.31 | 1.94 (1.96) | 0.0150487100 | 0.5759991 |
| 127848 | 3280.46 | 22.7334 | 0.19 | 2.57 (2.69) | 0.39 | 2.71 (2.72) | 0.0023150400 | 0.6013499 |
| 127852 | 3280.56 | 25.8155 | 0.58 | 2.49 (2.54) | 0.74 | 2.59 (2.63) | 0.0056296170 | 0.6128295 |
| 128086 | 3286.56 | 30.9207 | 0.29 | 3.13 (3.20) | 0.29 | 3.35 (3.41) | 0.0028173745 | 0.6395355 |
| 128435 | 3295.53 | 25.4528 | 0.24 | 2.08 (2.13) | 0.53 | 2.34 (2.35) | 0.0000051320 | 0.6662415 |
| 128525 | 3298.46 | 36.0541 | 0.2 | 2.11 (2.19) | 0.4 | 2.27 (2.40) | 0.0015579020 | 0.6065051 |
| 128650 | 3302.59 | 23.2932 | 0.34 | 2.17 (2.09) | 0.58 | 2.16 (2.14) | 0.0010580520 | 0.6248937 |
| 128936 | 3311.48 | 24.331 | 0.44 | 2.19 (2.22) | 0.24 | 2.11 (2.15) | 0.0023782750 | 0.6066645 |
| 129019 | 3314.43 | 20.1405 | 0.36 | 2.85 (2.97) | 0.53 | 2.89 (2.96) | 0.0112120600 | 0.5959290 |
| 129131 | 3318.55 | 30.992 | 0.27 | 2.03 (2.01) | 0.43 | 2.11 (2.06) | 0.0106985900 | 0.5900298 |
| 129182 | 3320.51 | 24.2533 | 0.22 | 2.02 (1.93) | 0.16 | 1.94 (1.92) | 0.0053500758 | 0.6134673 |
| 129821 | 3333.36 | 19.4182 | 0.05 | 2.59 (2.80) | 0.33 | 2.31 (2.28) | 0.0000015616 | 0.6369048 |
| 130044 | 3336.51 | 39.3802 | 0.18 | 2.78 (2.95) | 0.45 | 2.97 (3.14) | 0.0000659767 | 0.6365859 |
| 130077 | 3337.45 | 22.8134 | 0.21 | 2.37 (2.22) | 0.45 | 2.71 (2.75) | 0.0000880379 | 0.6363202 |
| 130432 | 3349.42 | 35.8938 | 0.24 | 2.15 (2.05) | 0.42 | 2.39 (2.44) | 0.0033692810 | 0.6019345 |
| 130482 | 3350.55 | 31.0174 | 0.21 | 2.05 (2.06) | 0.36 | 2.03 (2.04) | 0.0234200800 | 0.5754145 |
| 130747 | 3359.58 | 31.8979 | 0.79 | 2.88 (2.96) | 0.9 | 2.96 (3.04) | 0.0136373200 | 0.6023597 |
| 130803 | 3361.56 | 24.2435 | 0.16 | 2.19 (2.21) | 0.31 | 2.13 (2.20) | 0.0205089400 | 0.5718537 |
| 131294 | 3375.57 | 31.9169 | 0.81 | 2.89 (2.98) | 0.9 | 2.88 (2.93) | 0.0119721545 | 0.5826956 |
| 131548 | 3384.58 | 30.8333 | 0.35 | 2.02 (1.97) | 0.17 | 2.00 (1.91) | 0.0034353690 | 0.5935374 |
| 131589 | 3385.55 | 25.4887 | 0.64 | 3.14 (3.23) | 0.69 | 3.41 (3.48) | 0.0126442400 | 0.6017219 |
| 131932 | 3399.35 | 42.1045 | 0.32 | 2.21 (2.14) | 0.53 | 2.26 (2.33) | 0.0026036570 | 0.6125106 |
| 132014 | 3400.58 | 31.185 | 0.48 | 2.21 (2.25) | 0.31 | 2.16 (2.13) | 0.0125378400 | 0.5917304 |
| 132057 | 3401.66 | 23.4928 | 0.4 | 2.75 (2.82) | 0.67 | 2.99 (3.09) | 0.0000054511 | 0.6789966 |
| 133168 | 3432.59 | 32.0468 | 0.88 | 2.78 (2.80) | 0.9 | 2.93 (3.02) | 0.0099935480 | 0.6070366 |
| 133345 | 3439.6 | 39.225 | 0.51 | 2.39 (2.42) | 0.32 | 2.26 (2.36) | 0.0038071550 | 0.6077806 |
| 134053 | 3462.35 | 19.3741 | 0.1 | 2.47 (2.48) | 0.38 | 2.45 (2.49) | 0.0000108821 | 0.6363733 |
| 134635 | 3478.43 | 41.7414 | 0.23 | 2.27 (2.25) | 0.52 | 2.73 (2.69) | 0.0000019993 | 0.6719813 |
| 135412 | 3510.6 | 40.2442 | 0.42 | 2.26 (2.31) | 0.65 | 2.27 (2.34) | 0.0021452610 | 0.6208546 |
| 135676 | 3520.61 | 30.7976 | 0.36 | 2.37 (2.38) | 0.5 | 2.38 (2.50) | 0.0260390500 | 0.5834396 |
| 136129 | 3536.46 | 20.2362 | 0.18 | 2.23 (2.13) | 0.4 | 2.10 (2.03) | 0.0021832140 | 0.6019345 |
| 136403 | 3546.67 | 26.2234 | 0.19 | 2.40 (2.26) | 0.35 | 2.44 (2.43) | 0.0111910300 | 0.5828019 |
| 136697 | 3556.59 | 23.9547 | 0.54 | 2.71 (2.71) | 0.75 | 2.87 (2.91) | 0.0002396327 | 0.6492347 |
| 136698 | 3556.6 | 22.636 | 0.35 | 3.21 (3.26) | 0.15 | 2.73 (2.78) | 0.0005487402 | 0.6088435 |
| 136789 | 3559.71 | 26.5191 | 0.23 | 2.33 (2.38) | 0.43 | 2.27 (2.33) | 0.0062460600 | 0.5950787 |
| 136790 | 3559.71 | 24.9213 | 0.59 | 2.85 (2.89) | 0.77 | 2.91 (2.96) | 0.0055498310 | 0.6134141 |
| 137489 | 3582.7 | 19.465 | 0.69 | 3.24 (3.23) | 0.59 | 2.99 (3.05) | 0.0079915760 | 0.6077806 |
| 137922 | 3583.64 | 41.4711 | 0.23 | 2.41 (2.54) | 0.56 | 2.51 (2.56) | 0.0000062693 | 0.6659226 |
| 138036 | 3589.68 | 25.0338 | 0.66 | 2.66 (2.65) | 0.77 | 2.77 (2.76) | 0.0230354000 | 0.5934311 |
| 138067 | 3590.7 | 29.0044 | 0.07 | 1.95 (1.90) | 0.4 | 2.20 (2.29) | 0.0000000560 | 0.6669855 |
| 138143 | 3593.47 | 20.1962 | 0.26 | 2.59 (2.57) | 0.52 | 2.59 (2.62) | 0.0002780708 | 0.6325468 |
| 138813 | 3605.57 | 21.1852 | 0.22 | 2.30 (2.31) | 0.39 | 2.39 (2.46) | 0.0093466580 | 0.5880102 |
| 139064 | 3616.72 | 33.188 | 0.57 | 2.34 (2.37) | 0.7 | 2.54 (2.58) | 0.0005235135 | 0.6406250 |
| 139455 | 3630.44 | 21.7771 | 0.42 | 2.64 (2.66) | 0.69 | 2.56 (2.60) | 0.0033100550 | 0.6164966 |
| 139554 | 3632.74 | 33.2296 | 0.12 | 1.91 (1.93) | 0.33 | 1.97 (2.01) | 0.0003575735 | 0.6088435 |
| 140112 | 3657.67 | 40.7109 | 0.7 | 2.90 (2.95) | 0.83 | 3.11 (3.15) | 0.0014098810 | 0.6319090 |
| 140570 | 3677.77 | 24.4986 | 0.21 | 2.56 (2.54) | 0.4 | 2.83 (2.96) | 0.0019449460 | 0.6049107 |
| 140672 | 3681.72 | 32.0178 | 0.2 | 1.89 (1.92) | 0.4 | 1.94 (1.99) | 0.0024630710 | 0.6019345 |
| 140780 | 3685.83 | 22.1964 | 0.59 | 2.92 (3.01) | 0.67 | 3.09 (3.20) | 0.0196594800 | 0.5944940 |
| 141007 | 3696.76 | 26.9428 | 0.11 | 2.21 (2.28) | 0.39 | 2.29 (2.27) | 0.0000101960 | 0.6389775 |
| 141019 | 3697.56 | 23.7039 | 0.23 | 2.27 (2.12) | 0.38 | 2.37 (2.43) | 0.0228703800 | 0.5770621 |
| 141109 | 3701.74 | 32.4441 | 0.21 | 2.08 (2.08) | 0.4 | 2.17 (2.14) | 0.0035774690 | 0.5986395 |
| 141672 | 3718.72 | 32.4816 | 0.79 | 2.33 (2.36) | 0.81 | 2.67 (2.67) | 0.0004001464 | 0.6465774 |
| 142080 | 3734.72 | 32.4972 | 0.68 | 2.20 (2.22) | 0.77 | 2.50 (2.52) | 0.0001800037 | 0.6541241 |
| 142141 | 3737.66 | 37.205 | 0.21 | 2.04 (1.98) | 0.39 | 2.05 (2.11) | 0.0090732330 | 0.5878508 |
| 143120 | 3765.6 | 20.1729 | 0.2 | 2.31 (2.24) | 0.36 | 2.34 (2.37) | 0.0101199900 | 0.5850340 |
| 143333 | 3774.72 | 22.9371 | 0.36 | 3.15 (3.15) | 0.18 | 2.33 (2.38) | 0.0007108390 | 0.6097470 |
| 143379 | 3775.75 | 25.5852 | 0.84 | 2.84 (2.79) | 0.84 | 3.06 (3.11) | 0.0196265000 | 0.5968325 |
| 143652 | 3788.82 | 25.1861 | 0.22 | 2.01 (1.97) | 0.52 | 2.16 (2.17) | 0.0000100722 | 0.6591199 |
| 143947 | 3801.77 | 33.4628 | 0.43 | 2.17 (2.12) | 0.66 | 2.34 (2.39) | 0.0000993604 | 0.6538053 |
| 144344 | 3817.79 | 33.5359 | 0.14 | 1.87 (1.89) | 0.3 | 1.87 (1.95) | 0.0086312710 | 0.5794005 |
| 144635 | 3831.81 | 28.4845 | 0.33 | 2.43 (2.48) | 0.66 | 2.69 (2.63) | 0.0000014642 | 0.6864371 |
| 145238 | 3858.84 | 25.8539 | 0.53 | 2.94 (3.04) | 0.73 | 3.01 (3.05) | 0.0034901850 | 0.6183036 |
| 145479 | 3871.79 | 27.5695 | 0.3 | 2.37 (2.47) | 0.56 | 2.30 (2.42) | 0.0007240963 | 0.6267007 |
| 146624 | 3927.82 | 33.5971 | 0.2 | 2.05 (2.04) | 0.33 | 2.23 (2.30) | 0.0142028100 | 0.5795068 |
| 146936 | 3943.83 | 33.6285 | 0.44 | 2.11 (2.11) | 0.65 | 2.17 (2.23) | 0.0023510860 | 0.6202168 |
| 146964 | 3944.71 | 24.5544 | 0.29 | 2.18 (2.07) | 0.5 | 2.23 (2.34) | 0.0011327180 | 0.6191539 |
| 147000 | 3945.91 | 22.0304 | 0.19 | 2.56 (2.69) | 0.31 | 2.94 (2.90) | 0.0180686800 | 0.5750425 |
| 147376 | 3959.69 | 19.9025 | 0.19 | 2.67 (2.67) | 0.42 | 2.60 (2.72) | 0.0010706830 | 0.6107568 |
| 147541 | 3968.6 | 21.0938 | 0.63 | 3.01 (3.09) | 0.85 | 3.33 (3.41) | 0.0000000071 | 0.7385204 |
| 148086 | 3986.65 | 20.6016 | 0.81 | 3.42 (3.42) | 0.91 | 3.58 (3.68) | 0.0003926429 | 0.6471620 |
| 148557 | 4002.62 | 20.6566 | 0.36 | 2.60 (2.62) | 0.65 | 2.90 (2.93) | 0.0000010960 | 0.6895196 |
| 148717 | 4008.81 | 23.4219 | 0.4 | 2.86 (2.71) | 0.13 | 2.26 (2.33) | 0.0000079140 | 0.6438138 |
| 149142 | 4025.6 | 20.784 | 0.24 | 2.24 (2.35) | 0.45 | 2.50 (2.54) | 0.0006526890 | 0.6202168 |
| 149624 | 4043.64 | 20.3849 | 0.63 | 2.85 (2.88) | 0.74 | 3.08 (3.08) | 0.0023156390 | 0.6246811 |
| 149760 | 4044.92 | 26.3671 | 0.78 | 3.23 (3.27) | 0.88 | 3.43 (3.44) | 0.0030442670 | 0.6228741 |
| 150139 | 4059.65 | 20.4488 | 0.13 | 2.65 (2.70) | 0.38 | 2.59 (2.65) | 0.0001599918 | 0.6197917 |
| 150441 | 4062.8 | 33.1373 | 0.6 | 2.74 (2.76) | 0.43 | 2.23 (2.35) | 0.0000084792 | 0.6742666 |
| 150781 | 4078.81 | 33.1371 | 0.61 | 2.47 (2.54) | 0.41 | 1.98 (2.03) | 0.0000030088 | 0.6823980 |
| 151244 | 4097.87 | 24.6106 | 0.62 | 2.51 (2.41) | 0.81 | 2.88 (2.94) | 0.0000102206 | 0.6813350 |
| 152744 | 4160.92 | 25.8191 | 0.56 | 3.49 (3.58) | 0.41 | 3.29 (3.39) | 0.0060558040 | 0.6060268 |
| 152967 | 4169.93 | 33.5832 | 0.82 | 2.72 (2.83) | 0.85 | 2.97 (3.03) | 0.0013607440 | 0.6328656 |
| 153832 | 4196.75 | 20.8379 | 0.38 | 2.29 (2.26) | 0.7 | 2.48 (2.55) | 0.0000003345 | 0.7012649 |
| 155132 | 4251.98 | 28.7652 | 0.53 | 2.36 (2.37) | 0.69 | 2.75 (2.82) | 0.0000121296 | 0.6763393 |
| 156081 | 4289.93 | 28.7838 | 0.46 | 3.25 (3.35) | 0.74 | 3.46 (3.53) | 0.0000008441 | 0.6979698 |
| 156445 | 4305.94 | 28.8296 | 0.86 | 3.11 (3.22) | 0.96 | 3.34 (3.40) | 0.0000309130 | 0.6731505 |
| 156878 | 4321.94 | 25.1991 | 0.33 | 2.98 (3.16) | 0.11 | 2.57 (2.34) | 0.0002044592 | 0.6122449 |
| 157882 | 4352.86 | 20.1681 | 0.83 | 3.51 (3.52) | 0.91 | 3.63 (3.75) | 0.0143665700 | 0.6016688 |
| 158341 | 4368.9 | 20.2129 | 0.24 | 2.87 (2.87) | 0.56 | 2.96 (2.97) | 0.0000089759 | 0.6636905 |
| 159259 | 4404.84 | 20.6659 | 0.48 | 2.76 (2.82) | 0.7 | 2.94 (2.96) | 0.0001228249 | 0.6539647 |
| 159396 | 4409.89 | 20.001 | 0.48 | 2.88 (2.88) | 0.72 | 3.01 (3.10) | 0.0003120915 | 0.6449298 |
| 160089 | 4436.08 | 26.3177 | 0.41 | 2.92 (3.10) | 0.53 | 3.33 (3.38) | 0.0056652660 | 0.6060799 |
| 167786 | 4771.07 | 20.1987 | 0.4 | 2.80 (2.78) | 0.6 | 2.89 (2.99) | 0.0012013070 | 0.6259566 |
| 173077 | 5213.09 | 22.4341 | 0.39 | 2.29 (2.26) | 0.67 | 2.31 (2.37) | 0.0002834637 | 0.6426446 |
| 174387 | 5427.17 | 22.935 | 0.21 | 2.21 (2.24) | 0.41 | 2.04 (2.03) | 0.0102440500 | 0.5874256 |
| 174987 | 5511.28 | 22.4639 | 0.19 | 1.96 (1.83) | 0.34 | 2.10 (2.18) | 0.0109095200 | 0.5825361 |
| 175094 | 5528.35 | 28.0044 | 0.34 | 2.39 (2.51) | 0.19 | 2.19 (2.24) | 0.0086815890 | 0.5845026 |
| 175343 | 5574.25 | 23.2009 | 0.56 | 2.87 (2.85) | 0.89 | 2.85 (2.85) | 0.0005059226 | 0.6430166 |
| 177848 | 6211.74 | 20.2851 | 0.2 | 2.44 (2.44) | 0.53 | 2.67 (2.70) | 0.0000005482 | 0.6797406 |
| 177971 | 6236.91 | 21.066 | 0.78 | 3.12 (3.09) | 0.72 | 2.92 (2.90) | 0.0084425710 | 0.6086310 |
| 178819 | 6541.76 | 20.6784 | 0.22 | 2.61 (2.57) | 0.42 | 2.52 (2.51) | 0.0078358400 | 0.5915179 |
| 179035 | 6650.64 | 25.5371 | 0.24 | 2.44 (2.51) | 0.47 | 2.40 (2.39) | 0.0030185160 | 0.6055485 |
| 181018 | 7906.95 | 19.5282 | 0.18 | 2.55 (2.55) | 0.36 | 2.50 (2.48) | 0.0073611890 | 0.5874787 |
| 181250 | 7958.47 | 34.3896 | 0.14 | 2.47 (2.41) | 0.38 | 2.52 (2.50) | 0.0002119928 | 0.6184099 |
| 184099 | 8853.77 | 21.097 | 0.15 | 2.90 (2.88) | 0.42 | 2.96 (2.99) | 0.0000642389 | 0.6321216 |
| 184206 | 8917.25 | 22.5451 | 0.34 | 2.51 (2.48) | 0.61 | 2.49 (2.47) | 0.0005726299 | 0.6323873 |
| 186609 | 10999.9 | 21.3719 | 0.12 | 3.25 (3.26) | 0.31 | 3.16 (3.16) | 0.0018166520 | 0.5934311 |
| 188895 | 11967.6 | 20.4687 | 0.38 | 2.60 (2.61) | 0.4 | 2.56 (2.46) | 0.0031000805 | 0.5982674 |
| 190524 | 14109.5 | 21.9308 | 0.27 | 2.90 (2.94) | 0.49 | 2.81 (2.83) | 0.0043835090 | 0.6031037 |

HI = Heart Failure
AUC = Area Under the Curve

Specificity is defined as the number of actually negative samples divided by the sum of the numbers of the actually negative and false positive samples. A specificity of 100% means that a test recognizes all healthy persons as being healthy, i.e., no healthy subject is identified as being ill. This says nothing about how reliably the test recognizes sick patients.

Sensitivity is defined as the number of actually positive samples divided by the sum of the numbers of the actually positive and false negative samples. A sensitivity of 100% means that the test recognizes all sick persons. This says nothing about how reliably the test recognizes healthy subjects.

By the markers according to the invention, it is possible to detect heart failure with a specificity of at least 60%, preferably at least 70%, more preferably 80%, even more preferably at least 90% and most preferably at least 95%.

By the markers according to the invention, it is possible to detect heart failure with a sensitivity of at least 60%, preferably at least 70%, more preferably 80%, even more preferably at least 90% and most preferably at least 95%.

The migration time is determined by capillary electrophoresis (CE), for example, as set forth in the Example under item 2. Thus, a glass capillary of 90 cm in length and with an inner diameter (ID) of 50 μm and an outer diameter (OD) of 360 μm is operated at an applied voltage of 30 kV. As the solvent, 30% methanol, 0.5% formic acid in water is used, for example.

It is known that the CE migration times may vary. Nevertheless, the order in which the polypeptide markers are eluted is typically the same for any CE system employed. In order to balance the differences in the migration time, the system may be normalized using standards for which the migration times are known. These standards may be, for example, the polypeptides stated in the Examples (see the Example, item 3), or certain peptides known from urine, such as those described in Jantos-Siwy et al. (Quantitative Urinary Proteome Analysis for Biomarker Evaluation in Chronic Kidney Disease. J. Proteome. Res. 8, 268-281 (2009)).

The characterization of the polypeptides shown in Table 1 was determined by means of capillary electrophoresis-mass spectrometry (CE-MS), a method which has been described in detail, for example, by Neuhoff et al. (Rapid Communications in mass spectrometry, 2004, Vol. 20, pp. 149-156), Mischak, H. et al. (Capillary electrophoresis-mass spectrometry as a powerful tool in biomarker discovery and clinical diagnosis: An update of recent developments. Mass Spectrom. Rev. 28, 703-724 (2009)), Mischak, H. et al. (Comprehensive human urine standards for comparability and standardization in clinical proteome analysis. Proteomics Clin Appl. 4, 464-478 (2010)), Good, D. M. et al. (Naturally occurring human urinary peptides for use in diagnosis of chronic kidney disease. Mol. Cell Proteomics 9, 2424-2437 (2010)). The variation of the molecular masses between individual measurements or between different mass spectrometers is relatively small, typically within a range of ±0.05%, more preferably ±0.03%, even more preferably ±0.01% or 0.005%, when calibration is exact.

The polypeptide markers according to the invention are proteins or peptides or degradation products of proteins or peptides. They may be chemically modified, for example, by posttranslational modifications, such as glycosylation, phosphorylation, alkylation or disulfide bridges, or by other reactions, for example, within the scope of the degradation. Proceeding from the parameters that determine the polypeptide markers (molecular weight and migration time), it is possible to identify the sequence of the corresponding polypeptides by methods known in the prior art.

The polypeptides according to the invention are used to diagnose heart failure. “Diagnosis” means the process of knowledge gaining by assigning symptoms or phenomena to a disease or injury. The presence or absence of a polypeptide marker can be measured by any method known in the prior art. Methods which may be known are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or empirically.

In the context of the present invention, the threshold value is considered to be exceeded preferably if the measured value of the sample for a certain molecular mass is at least twice as high as that of a blank sample (for example, only buffer or solvent). The polypeptide marker or markers is/are used in such a way that its/their presence or absence is measured, wherein the presence or absence is indicative of heart failure. Thus, there are polypeptide markers which are typically present in subjects with heart failure, but occur less frequently or are absent in subjects with no heart failure. Further, there are polypeptide markers which are present in patients with heart failure, but are less frequently or not at all present in patients with no heart failure. The frequency at which a marker occurs in the group with heart failure or in the control group is stated as “frequency” in Table 2.

In addition or also alternatively to the frequencies (determination of presence or absence), the amplitudes may also be used for diagnosis. The amplitudes are used in such a way that the presence or absence is not critical, but the height of the signal (the amplitude) decides if the signal is present in both groups. A normalization method is recommendable in order to achieve comparability between samples of different concentrations or between different measuring methods. Preferably, collagen fragments are used, as described in detail by Jantos Siwy et al. (Quantitative Urinary Proteome Analysis for Biomarker Evaluation in Chronic Kidney Disease. J. Proteome. Res. 8, 268-281 (2009)). The linear regression is formed between the reference values for the amplitudes of the given known peptides (so-called “housekeping peptides”) and the experimentally established values. The slope of the regression line just corresponds to the relative concentration and is used as a normalization factor for calibrating all the peptide signals of this sample with a common normalization factor.

The decision for a diagnosis is made as a function of how high the amplitude of the respective polypeptide markers in the patient sample is in comparison with the mean amplitudes in the control groups or the “ill” group. If the amplitude rather corresponds to the mean amplitudes of the “ill” group, the existence of a vascular disease is to be considered, and if it rather corresponds to the mean amplitudes of the control group, the non-existence of a vascular disease is to be considered. The distance between the measured value and the mean amplitude can be considered a probability of the sample's belonging to a certain group. Alternatively, the distance between the measured value and the mean amplitude may be considered a probability of the sample's belonging to a certain group.

Preferably, both the frequency and the amplitude are used for evaluation. From measured data of an unknown sample, probabilities for classification into “heart failure” or “healthy” groups can be respectively derived, from which a total probability then results.

The Wilcox p value is a measure of the probability that the assignment of the markers to the two groups (heart failure and control) is based on a random distribution that is not correlated with heart failure. The smaller the Wilcox p value, the more probable is the correlation with heart failure.

The AUC value is a measure of the significance of the marker data; for an AUC value of 0.5, the marker data would be of no significance; for an AUC value of 1, the marker would be capable of distinguishing between the two groups with a certainty of 100%.

The subject from which the sample in which the presence or absence or amplitude of one or more polypeptide markers is determined is derived may be any subject which is capable of suffering from heart failure. Preferably, the subject is a mammal, and most preferably, it is a human.

In a preferred embodiment of the invention, not just three polypeptide markers, but a larger combination of polypeptide markers are used. By comparing a plurality of polypeptide markers, a bias in the overall result from a few individual deviations from the typical presence probability in single individuals can be reduced or avoided.

The sample in which the presence or absence of the peptide marker or markers according to the invention is measured may be any sample which is obtained from the body of the subject. The sample is a sample which has a polypeptide composition suitable for providing information about the state of the subject. For example, it may be blood, urine, synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample. In a preferred embodiment, the sample is a urine sample.

Urine samples can be taken as preferred in the prior art. Preferably, a midstream urine sample is used as said urine sample in the context of the present invention. For example, the urine sample may also be taken by means of a urination apparatus as described in WO 01/74275.

The presence or absence or amplitude of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands or specific probes, such as antibodies.

If required or desirable, the sample from the subject, for example, the urine sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, electrophoretic separation, i.e., separation by different migration behaviors of electrically charged particles in solution upon application of an electric field. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kD protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas MALDI (matrix-assisted laser desorption/ionization) is used for measuring ions from a sample crystallized in a matrix. To analyze the ions formed, quadrupoles, ion traps or time-of-flight (TOF) analyzers may be used, for example.

In electrospray ionization (ESI), the molecules present in solution are atomized, inter alia, under the influence of high voltage (e.g., 1-8 kV), which forms charged droplets at first that become smaller from the evaporation of the solvent. Finally, so-called Coulomb explosions result in the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a good resolution.

Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC MS (liquid chromatography/mass spectrometry), 2D-PAGE/MS and capillary electrophoresis-mass spectrometry (CE-MS). All the methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J. Chromatogr A, 2003, Vol. 1013: 157-171, and Electrophoresis, 2004, 25: 2044-2055) and in Wittke et al. (J. Chromatogr. A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared, and this pattern can be compared with reference patterns of a sick or healthy subjects. In most cases, it is sufficient to use a limited number of polypeptide markers for the diagnosis of UAS. A CE-MS method which includes CE coupled on-line to an ESI-TOF MS is further preferred.

For CE-MS, the use of volatile solvents is preferred, and it is best to work under essentially salt-free conditions. Examples of such solvents include acetonitrile, methanol and the like. The solvents can be diluted with water or a weak acid (e.g., 0.1% to 1% formic acid) in order to protonate the analyte, preferably the polypeptides.

By means of capillary electrophoresis, it is possible to separate molecules by their charge and size. Neutral particles will migrate at the speed of the electro-osmotic flow upon application of a current, while cations are accelerated towards the cathode, and anions are delayed. The advantage of the capillaries in electrophoresis resides in the favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are, for example, 30-100 cm. In addition, the separating capillaries are usually made of plastic-coated silica glass. The capillaries may be either untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the separation or altered meanwhile.

In a preferred method for measuring polypeptide markers, the markers of the sample are separated by capillary electrophoresis, then directly ionized and transferred on-line into a coupled mass spectrometer for detection. In the method according to the invention, it is advantageous to use several polypeptide markers for diagnosis. The use of at least 5, 6, 8 or 10 markers is preferred. In one embodiment, 20 to 50 markers are used.

In order to determine the probability of the existence of a disease when several markers are used, statistic methods known to the skilled person may be used. For example, the Random Forests method described by Weissinger et al. (Kidney Int., 2004, 65: 2426-2434) may be used by using a computer program such as S-Plus, or the support vector machines as described in the same publication. Further information can be taken from, for example, Dakna et al. BMC Bioinformatics 11 (2010) 594.

These methods combine several biomarkers into a single variable, which is associated with both the probability of the existence of the disease and with the severity of the disease. Thus, this variable can also be employed to determine the intensity or severity of the disease (for example, shown in Schiffer et al., Prediction of muscle-invasive bladder cancer using urinary proteomics. Clin. Cancer Res. 15, 4935-4943 (2009), for bladder carcinoma), and also to image any response to therapy (as shown, for example, in Haubitz et al., Identification and validation of urinary biomarkers for differential diagnosis and evaluation of therapeutic intervention in ANCA associated vasculitis. Mol. Cell. Proteomics 8, 2296-2307 (2009), for ANCA associated vasculitis, or in Andersen et al., Urinary proteome analysis enables assessment of renoprotective treatment in type 2 diabetic patients with microalbuminuria. BMC Nephrol. 11, 29 (2010), for diabetic nephropathy).

### EXAMPLE 1

1. Sample Preparation

For detecting the polypeptide markers for diagnosis, urine was employed. Urine was collected from healthy donors (control group) as well as from patients suffering from vascular diseases. For the subsequent CE-MS measurement, the proteins which are also contained in the urine of patients in an elevated concentration, such as albumin and immunoglobulins, had to be separated off by ultrafiltration. Thus, 700 μl of urine was collected and admixed with 700 μl of filtration buffer (2 M urea, 10 mM ammonia, 0.02% SDS). This 1.4 ml of sample volume was ultrafiltrated (20 kDa, Sartorius, Gottingen, Germany). The ultrafiltration was performed at 3000 rpm in a centrifuge until 1.1 ml of ultrafiltrate was obtained.

The 1.1 ml of filtrate obtained was then applied to a PD 10 column (Amersham Bioscience, Uppsala, Sweden) and eluted with 2.5 ml of 0.01% NH₄OH, and lyophilized. For the CE-MS measurement, the polypeptides were then resuspended with 20 μl of water (HPLC grade, Merck).

2. CE-MS Measurement

The CE-MS measurements were performed with a capillary electrophoresis system from Beckman Coulter (P/ACE MDQ System; Beckman Coulter Inc., Fullerton, Calif., USA) and an ESI-TOF mass spectrometer from Bruker (micro-TOF MS, Bruker Daltonik, Bremen, Germany). The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 30% isopropanol with 0.5% formic acid was used, here at a flow rate of 20 μl/min. The coupling of CE and MS was realized by a CE-ESI-MS Sprayer Kit (Agilent Technologies, Waldbronn, Germany). For injecting the sample, a pressure of from 1 to a maximum of 6 psi was applied, and the duration of the injection was 99 seconds. With these parameters, about 300 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi). After the separating voltage (25 kV) was applied, the analytes were automatically concentrated between these solutions. The subsequent CE separation was performed with a pressure method: 30 minutes at 0 psi, then 0.1 psi for 1 min, 0.2 psi for 1 min, 0.3 psi for 1 min, 0.4 psi for 1 min, and finally 0.5 psi for 35 min. The total duration of a separation run was thus 70 minutes.

In order to obtain as good a signal intensity as possible on the side of the MS, the nebulizer gas was turned to the lowest possible value. The voltage applied to the spray needle for generating the electrospray was 4000-4800 V. The remaining settings at the mass spectrometer were optimized for peptide detection according to the manufacturer's instructions. The spectra were recorded over a mass range of m/z 400 to m/z 3000 and accumulated every 3 seconds.

3. Standards for the CE Measurement

For checking and standardizing the CE measurement, the following proteins or polypeptides which are characterized by the stated CE migration times were employed:

| Protein/polypeptide | Migration time | Mass (Da) |
|---|---|---|
| Aprotinin (SIGMA, Taufkirchen, DE, | 19.3 min | 6513.09 |
| Cat. # A1153) | | |
| Ribonuclease (SIGMA, Taufkirchen, DE, | 19.55 min | 13681.32 |
| Cat. # R4875) | | |
| Lysozyme (SIGMA, Taufkirchen, DE, | 19.28 min | 14303.88 |
| Cat. # L7651) | | |
| “REV”, Sequence: REVQSKIGYGRQIIS | 20.95 min | 1732.96 |
| “ELM”, Sequence: | 23.49 min | 2333.19 |
| ELMTGELPYSHINNRDQIIFMVGR | | |
| “KINCON”, Sequence: | 22.62 min | 2832.41 |
| TGSLPYSHIGSRDQIIFMVGR | | |
| “GIVLY” Sequence: | 32.2 min | 2048.03 |
| GIVLYELMTGELPYSHIN | | |

The proteins/polypeptides were employed at a concentration of 10 pmol/μl each in water. “REV”, “ELM, “KINCON” and “GIVLY” are synthetic peptides.

In principle, it is known to the skilled person that slight variations of the migration times may occur in separations by capillary electrophoresis. However, under the conditions described, the order of migration will not change. For the skilled person who knows the stated masses and CE times, it is possible without difficulty to assign their own measurements to the polypeptide markers according to the invention. For example, he may proceed as follows: At first, he selects one of the polypeptides found in his measurement (peptide 1) and tries to find one or more identical masses within a time slot of the stated CE time (for example, ±5 min). If only one identical mass is found within this interval, the assignment is completed. If several matching masses are found, a decision about the assignment is still to be made. Thus, another peptide (peptide 2) from the measurement is selected, and it is tried to identify an appropriate polypeptide marker, again taking a corresponding time slot into account. Again, if several markers can be found with a corresponding mass, the most probable assignment is that in which there is a substantially linear relationship between the shift for peptide 1 and that for peptide 2. Depending on the complexity of the assignment problem, it suggests itself to the skilled person to optionally use further proteins from his sample for assignment, for example, ten proteins. Typically, the migration times are either extended or shortened by particular absolute values, or compressions or expansions of the whole course occur. However, comigrating peptides will also comigrate under such conditions.

In addition, the skilled person can make use of the migration patterns described by Zuerbig et al. in Electrophoresis 27 (2006), pp. 2111-2125. If he plots his measurement in the form of m/z versus migration time by means of a simple diagram (e.g., with MS Excel), the line patterns described also become visible. Now, a simple assignment of the individual polypeptides is possible by counting the lines. Other approaches of assignment are also possible. Basically, the skilled person could also use the peptides mentioned above as internal standards for assigning his CE measurements.

4. Sequencing of Polypeptides

The sequencing of the polypeptides determined by this process is basically known to the skilled person. The following method can be used.

The urine samples were analyzed according to Carty DM et al. (Urinary proteomics for prediction of preeclampsia. Hypertension. 2011; 57: 561-9) by means of a Dionex Ultimate 3000 RSLS Nanoflow System (Dionex, Camberley, UK) (LC/MS). The samples (5 μl) were charged at a flow rate of 5 μl/min in 0.1% formic acid and 2% acetonitrile onto a Dionex C18 Nano Trap Column (0.1×20 mm, 5 μm). The sample was eluted at a flow rate of 0.3 μl/min onto an Acclaim PepMap C18 Nanocolumn (75 μm×15 cm, 2 μm, 100 Å). The trap and nanoflow columns were maintained at 35° C. The samples were eluted over 100 min with a gradient of solvent A, 0.1% formic acid, against solvent B, acetonitrile, starting from 5% B and increasing to 50% B. The column was washed with 90% B before being equilibrated for the next sample. The eluent flowing from the column was ionized into an Orbitrap Velos FTMS using a Proxeon Nanospray ESI source (Thermo Fisher, Hemel Hempstead, UK) working in a positive ion mode. The ionization voltage was 2.5 kV, and the capillary temperature was 200° C. The mass spectrometer was operated in an MS/MS mode with a scanning range of from m/z 380 to 2000 amu. The 10 largest multiply charged ions were selected from every scan for MS/MS analysis; the fragmenting method was HCD with 35% collision energy. The ions were selected by means of a data-dependent method with a repetition number of 1 and an exclusion time of 15 s for MS2. The ion resolution was 60000 in MS1 and 7500 for HCD-MS2. The files were used for a search against the human non-redundant data base IPI using the Open Mass Spectrometry Search Algorithm (OMSSA, http://pubchem.ncbi.nlm.nih.gov/omssa) and SEQUEST (using the Thermo Proteome Discoverer), without any enzyme specificity. No predetermined modification and oxidation of methionine and proline was chosen as variable modifications. The admissible mass error window for MS and MS/MS was 10 ppm or 0.05 Da. In the case of SEQUEST, the peptide data were extracted using high peptide confidence and Top-One peptide rank filters. 1% FDR was used as the threshold value for considering peptides as identified. The correlation between the peptide charge at a working pH of 2 and the CE migration time was utilized to minimize the occurrence of false positive identifications (Zurbig, P. et al.

Biomarker discovery by CE-MS enables sequence analysis via MS/MS with platform-independent separation. Electrophoresis 27, 2111-2125 (2006)). The CE migration time calculated on the basis of the number of basic amino acids was compared with the experimental migration time. Only those peptides were accepted that were found with both searching algorithms (OMSSA and

SEQUEST) and that had a mass deviation of less than ±10 ppm, fragment ions with a mass deviation of less than ±0.05 Da and a deviation of CE migration time of less than ±2 min.

### EXAMPLE 2

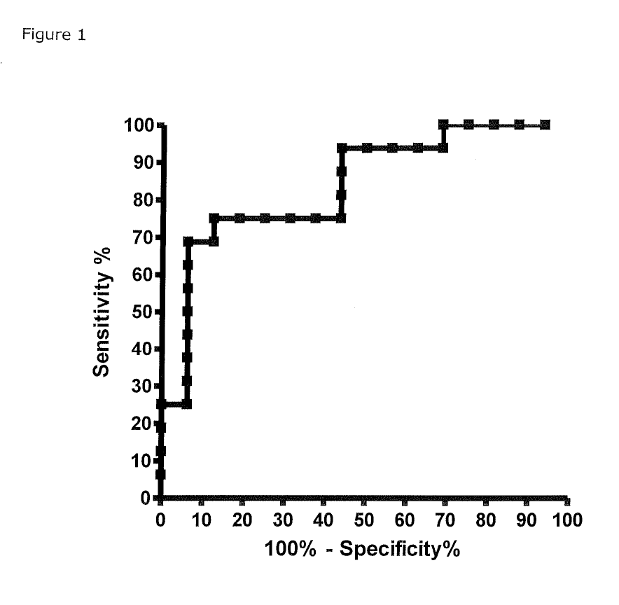

From the FLEMENGHO study, 16 patients with symptomatic heart failure and 16 healthy controls were selected, and urine samples were measured in blind tests. Based on the markers, a precision of the classification of 0.84 was obtained (95% interval 0.70 to 0.98, p=0.001; see FIG. 1).

### EXAMPLE 3

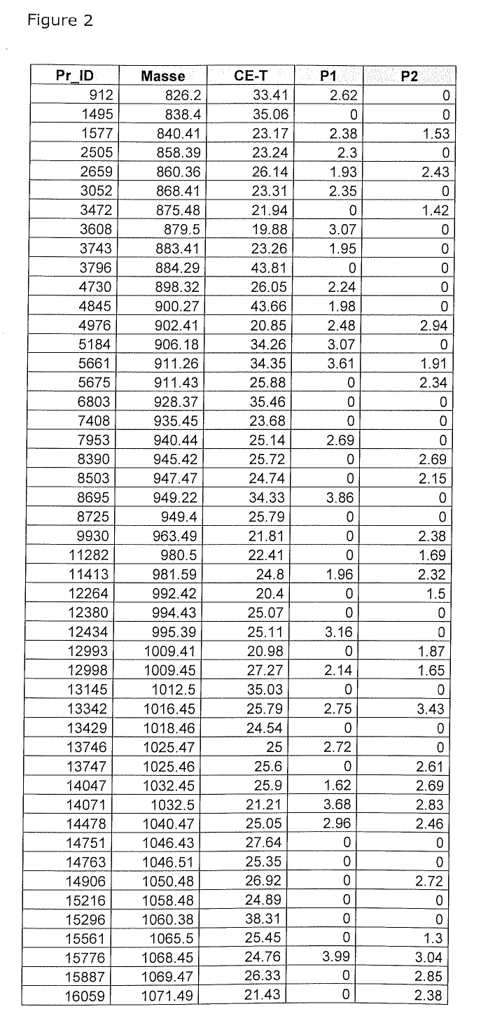

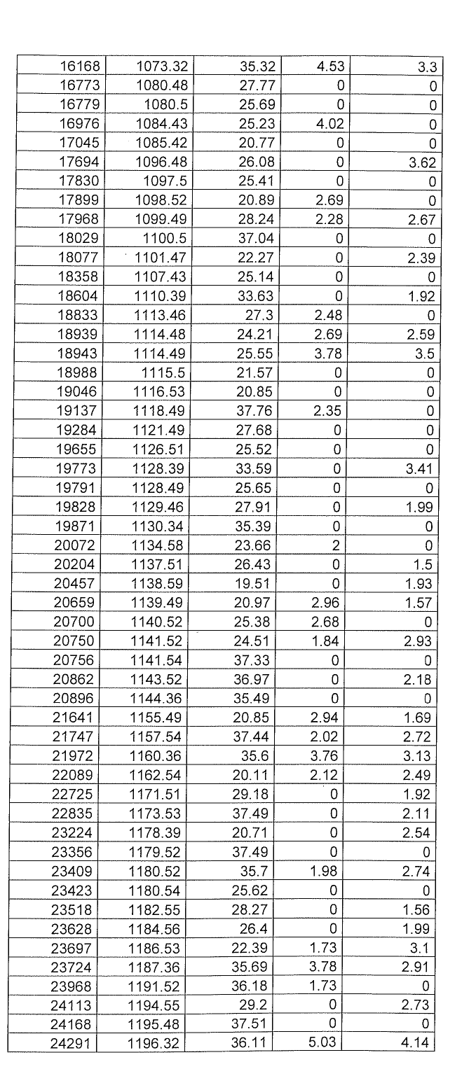

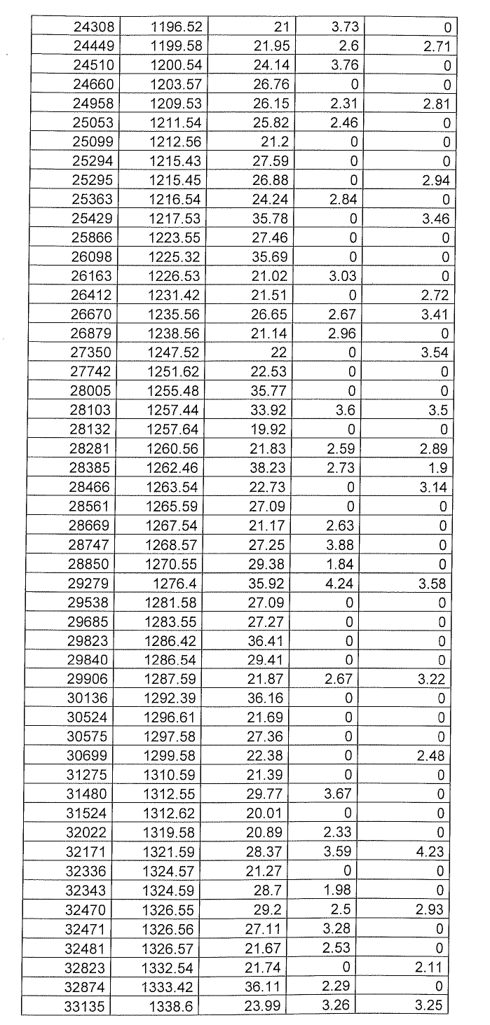

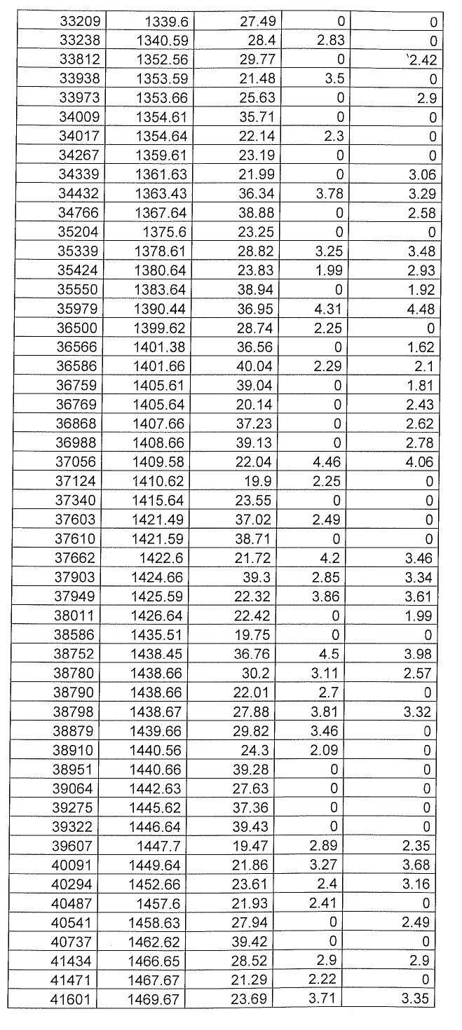

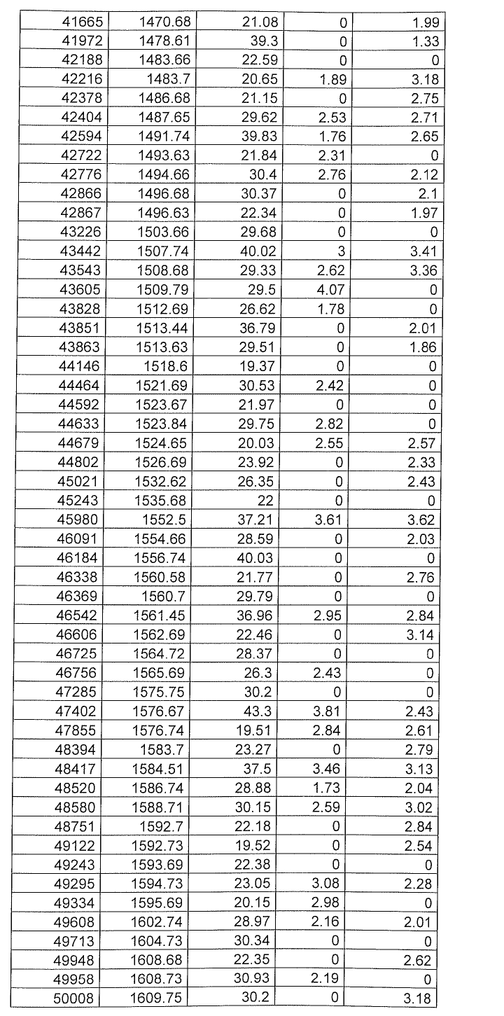

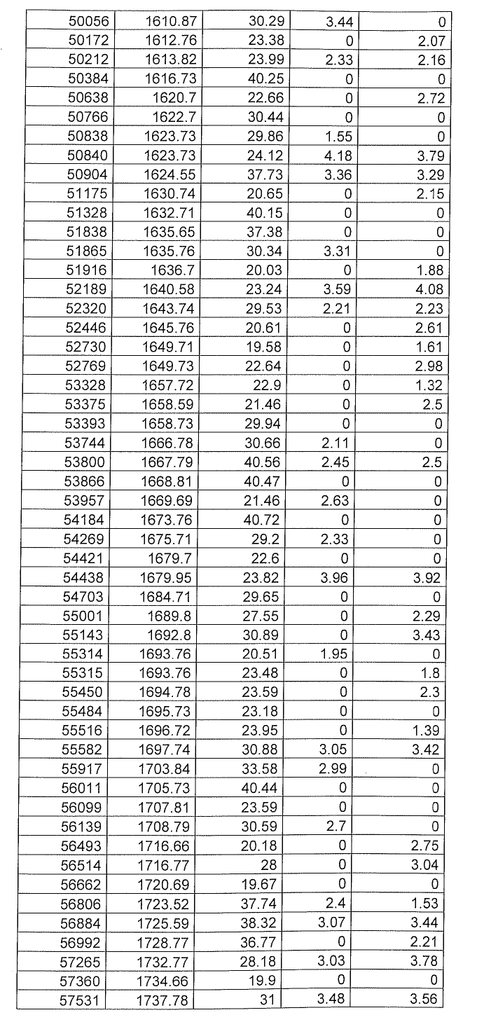

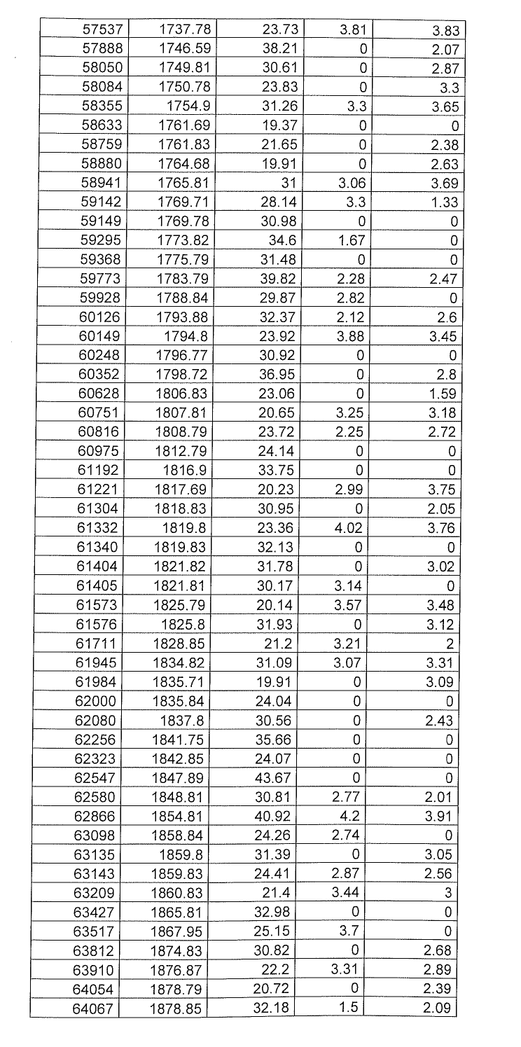

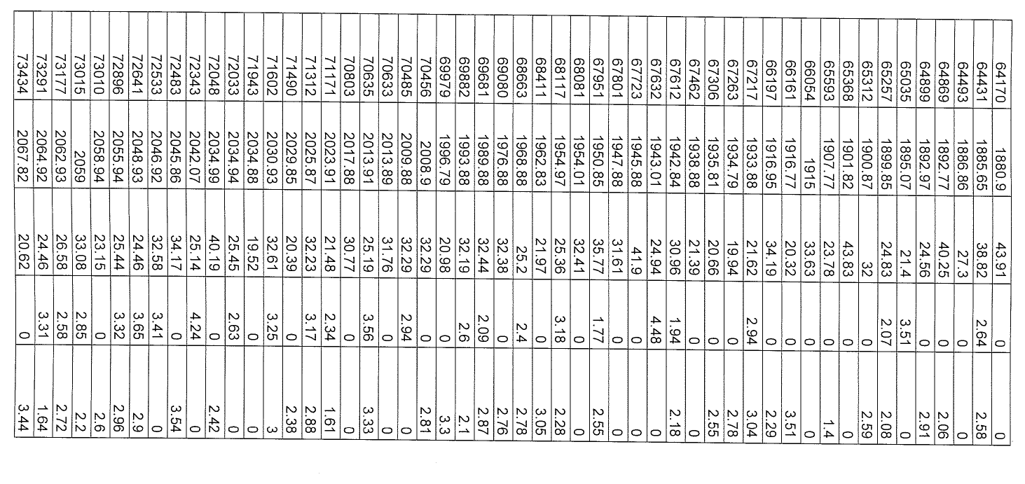

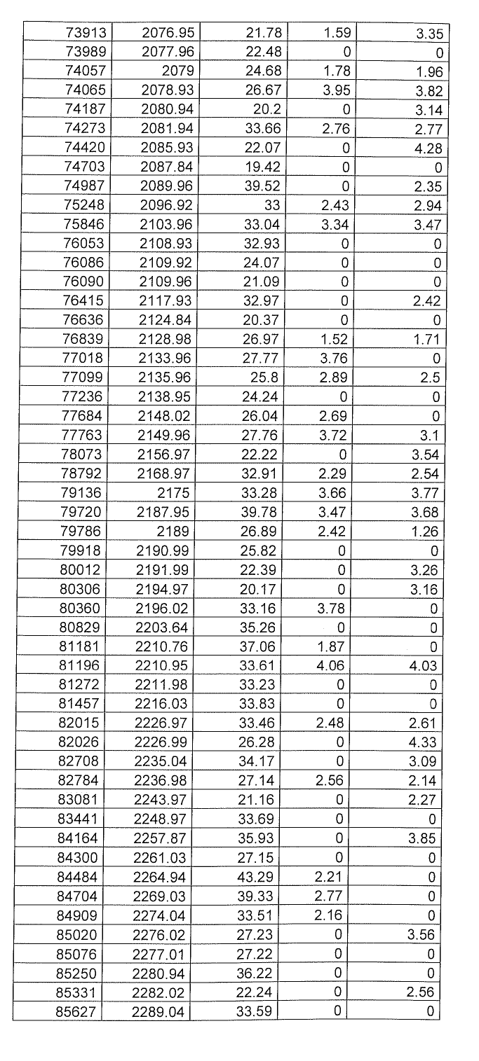

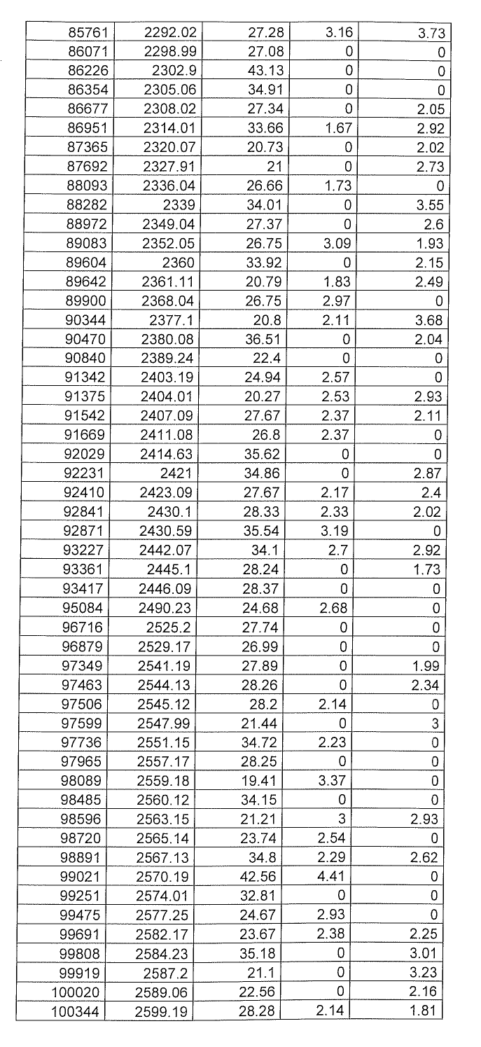

FIG. 2 shows measured values of the biomarkers according to the invention for two unknown samples. An amplitude of 0 means that the marker was not found. From the frequency and amplitude data, a score for the classification into heart failure and healthy groups was derived. A measured value belonging to the heart failure group as judged by the frequency/amplitude was given a positive score; a measured value belonging to the healthy group as judged by the frequency/amplitude was given a negative score. The closer the value was to the values of Table 2, the higher (absolutely) was the score. Subsequently, the individual score values were combined, wherein the Wilcox p values and AUC values also contributed to the assessment. For sample 1, a score value of 1.701 was obtained, and for sample 2, a score value of −0.853 was obtained.

Subsequently, the patients were examined: Subject 1 had a clinically not yet visible heart failure, while such signs were not found in subject 2.

### EXAMPLE 4

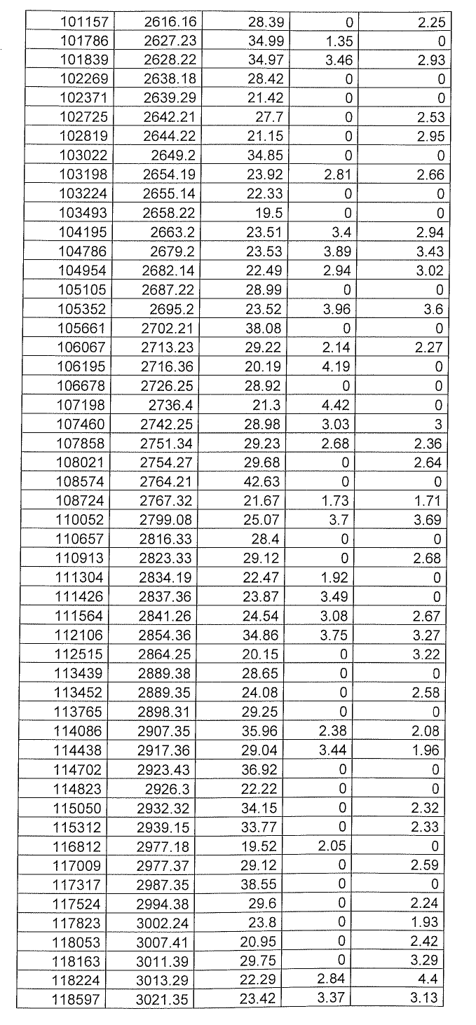

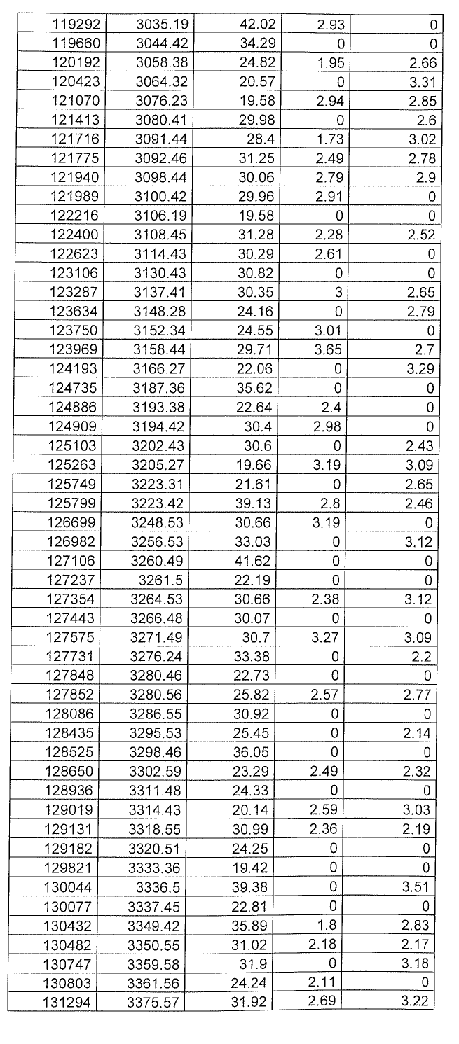

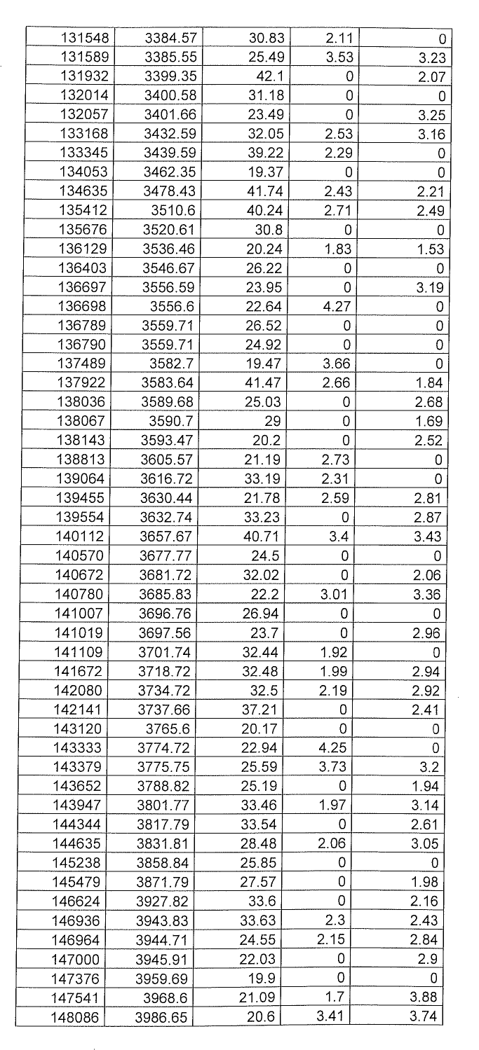

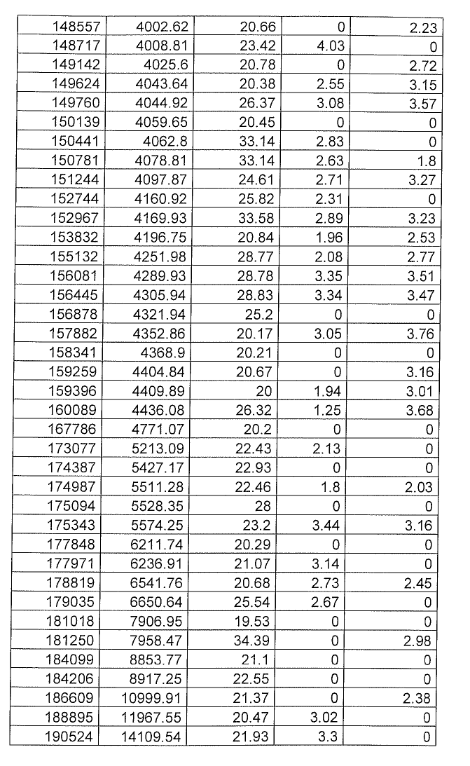

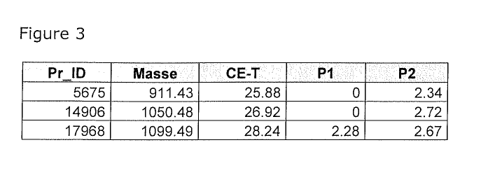

FIG. 3 shows measured values for the same subjects, in which only three markers were evaluated.

Subject 1: The markers with IDs of 5675 and 14906 were not found in the sample. Since these markers occur less frequently in the heart failure group, their absence means a positive score for heart failure. Marker 17968 occurs more frequently in the “healthy” group; therefore, the presence of the marker results in a negative score. However, the amplitude is very close to the average amplitude of the heart failure group, which is a strong positive score; an overall score of 1.063 is obtained.

For subject 2, a negative score is obtained because of the presence of marker 5675. The amplitude of 2.34 is between those of the heart failure and control groups, but slightly more closely to that of the heart failure group, which results in a small positive score.

For marker 14906, a negative score is obtained from its presence, and in addition, its amplitude is close to the average amplitude of the control group, so that a negative overall score is obtained.

From the presence of marker 17968, a negative score is obtained because of the frequencies; in addition, another classification into the control group and thus another negative score is obtained from the amplitude of 2.67.

All in all, a score of −1.185 is obtained for subject 2 from the three measured values.

The classifications of subjects 1 and 2 could be confirmed in a clinical examination.

1. A process for the diagnosis of heart failure, comprising the step of determining the presence or absence or amplitude of at least three polypeptide markers in a urine sample, wherein said polypeptide markers are selected from the markers characterized in Table 1 by values for the molecular masses and migration times.

2. The process according to claim 1, wherein an evaluation of the determined presence or absence or amplitude of the markers is effected by means of the reference values stated in Table 2.

3. The process according to claim 1, wherein at least five, at least six, at least eight, at least ten, at least 20 or at least 50 polypeptide markers as defined in claim 1 are used.

4. The process according claim 1, wherein said sample from a subject is a midstream urine sample.

5. The process according to claim 1, wherein capillary electrophoresis, HPLC, gas-phase ion spectrometry and/or mass spectrometry is used for detecting the presence or absence or amplitude of said polypeptide markers.

6. The process according to claim 1, wherein a capillary electrophoresis is performed before the molecular mass of said polypeptide markers is measured.

7. The process according to claim 1, wherein mass spectrometry is used for detecting the presence or absence of said polypeptide marker or markers.

8. (canceled)

9. A method for the diagnosis of heart failure comprising the steps:
a) separating a sample into at least five subsamples;
b) analyzing at least five subsamples for determining the presence or absence or amplitude of at least one polypeptide marker in the subsample, wherein said polypeptide marker is selected from the markers of Table 1, which are characterized by their molecular masses and migrations times (CE times).

10. The method according to claim 9, wherein at least 10 subsamples are measured.

11. The method according to claim 1, wherein the CE time is based on a 90 cm length glass capillary having an inner diameter (ID) of 50 μm at an applied voltage of 25 kV, wherein 20% acetonitrile, 0.25 M formic acid in water is used as the mobile solvent.

12. The method according to claim 1, wherein the sensitivity is at least 60% and the specificity is at least 40%.

13. The method according to claim 1, wherein said heart failure is a left-ventricular disorder.

14. The method according to claim 1, wherein said heart failure is a left-ventricular diastolic heart failure.

15. The method according to claim 1, wherein said heart failure is asymptomatic.

16. A method for evaluating the development of a heart failure, comprising the performance of the process according to claim 1 at two different times.

17. The method according to claim 16 for evaluating a therapeutic intervention.

18. A method for prognosing the development of a heart failure, comprising the step of determining the presence or absence or amplitude of at least three polypeptide markers in a urine sample, wherein said polypeptide markers are selected from the markers characterized in Table 1 by values for the molecular masses and migration times.

19. The method according to claim 18, wherein said heart failure is already clinically relevant.

20. The method according to claim 9, wherein the CE time is based on a 90 cm length glass capillary having an inner diameter (ID) of 50 μm at an applied voltage of 25 kV, wherein 20% acetonitrile, 0.25 M formic acid in water is used as the mobile solvent.

21. The method according to claim 9, wherein the sensitivity is at least 60% and the specificity is at least 40%.

22. The method according to claim 9, wherein said heart failure is a left-ventricular disorder.

23. The method according to claim 9, wherein said heart failure is a left-ventricular diastolic heart failure.

24. The method according to claim 9, wherein said heart failure is asymptomatic.

25. A method for evaluating the development of a heart failure, comprising the performance of the process according to claim 9 at two different times.

26. The method according to claim 25 for evaluating a therapeutic intervention.
